SEQUENCE LISTING (19 sequences)

SEQ ID NO: 1 - DNA, 39 bp, synthetic construct
  source 1..39: mol_type = other DNA
  modified_base 26: mod_base = i
  modified_base 27: mod_base = i
  modified_base 28: mod_base = i
  modified_base 29: mod_base = i
  modified_base 30: mod_base = i
tgatatccatcctaagactaatcgtnnnnnagttgaaac

SEQ ID NO: 2 - DNA, 35 bp, synthetic construct
  source 1..35: mol_type = other DNA
  modified_base 22: mod_base = i
  modified_base 23: mod_base = i
  modified_base 24: mod_base = i
  modified_base 25: mod_base = i
  modified_base 26: mod_base = i
ccaattacctttcctccatcgnnnnngctgagaaa

SEQ ID NO: 3 - DNA, 40 bp, synthetic construct
  source 1..40: mol_type = other DNA
cttcgatcgcgtcacggtgttggtgcatcagttgttaatg

SEQ ID NO: 4 - DNA, 38 bp, synthetic construct
  source 1..38: mol_type = other DNA
ggctcgcatagattcatggctgggtgacgcgatcgaag

SEQ ID NO: 5 - DNA, 28 bp, synthetic construct
  source 1..28: mol_type = other DNA
gtcacccagccatgaatctatgcgagcc

SEQ ID NO: 6 - DNA, 38 bp, synthetic construct
  source 1..38: mol_type = other DNA
  modified_base 4: mod_base = i
  modified_base 13: mod_base = i
cttngatcgcgtnacccagccatgaatctatgcgagcc

SEQ ID NO: 7 - DNA, 38 bp, synthetic construct; identical to SEQ ID NO: 4
  source 1..38: mol_type = other DNA

SEQ ID NO: 8 - DNA, 28 bp, synthetic construct
  source 1..28: mol_type = other DNA
cttcgatcgcgtcacccagccatgaatc

SEQ ID NO: 9 - DNA, 38 bp, synthetic construct
  source 1..38: mol_type = other DNA
  modified_base 26: mod_base = i
  modified_base 33: mod_base = i
cttcgatcgcgtcacccagccatgantctatgngagcc

SEQ ID NO: 10 - DNA, 447 bp, synthetic construct
  source 1..447: mol_type = other DNA
ctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccaca

SEQ ID NO: 11 - DNA, 26 bp, synthetic construct
  source 1..26: mol_type = other DNA
aaaccttttgagagagatatttcaac

SEQ ID NO: 12 - DNA, 26 bp, synthetic construct
  source 1..26: mol_type = other DNA
acatttgtttttaaccaaattagtag

SEQ ID NO: 13 - DNA, 46 bp, synthetic construct
  source 1..46: mol_type = other DNA
cctcaggtggcaatatggtgttggttaccaaccatacagagtagta

SEQ ID NO: 14 - DNA, 38 bp, synthetic construct
  source 1..38: mol_type = other DNA
cctttcgcccacatcgttgctgccattgccacctgagg

SEQ ID NO: 15 - DNA, 27 bp, synthetic construct
  source 1..27: mol_type = other DNA
aatggcagcaacgatgtgggcgaaagg

SEQ ID NO: 16 - DNA, 41 bp, synthetic construct
  source 1..41: mol_type = other DNA
  modified_base 27: mod_base = i
  modified_base 28: mod_base = i
  modified_base 29: mod_base = i
  modified_base 30: mod_base = i
  modified_base 31: mod_base = i
gcttcatgtactactaactgtttagcnnnnnttgccaacgt

SEQ ID NO: 17 - DNA, 36 bp, synthetic construct
  source 1..36: mol_type = other DNA
  modified_base 22: mod_base = i
  modified_base 23: mod_base = i
  modified_base 24: mod_base = i
  modified_base 25: mod_base = i
  modified_base 26: mod_base = i
tccaatagctattgcagcaccnnnnnttgttggaac

SEQ ID NO: 18 - DNA, 42 bp, synthetic construct
  source 1..42: mol_type = other DNA
atatcgcgcgtctgctccacacaaagatttaagaagagcaag

SEQ ID NO: 19 - DNA, 38 bp, synthetic construct
  source 1..38: mol_type = other DNA
tttatttatttatttattttactgcagacgcgcgatat

(19) United States
(12) Patent Application Publication
(10) Pub. No.: US 2026/0250745 A1
(43) Pub. Date: Aug. 27, 2026

(54) DETECTION OF TARGET NUCLEIC ACID BY LPHO-ASSISTED PTO CLEAVAGE AND EXTENSION ASSAY

(71) Applicant: SEEGENE, INC., Seoul (KR)

(72) Inventors: Jeong Woo KIM, Seoul (KR); Han Bit LEE, Seoul (KR); Yea Seong HA, Seoul (KR)

(73) Assignee: SEEGENE, INC., Seoul (KR)

(21) Appl. No.: 19/149,264

(22) PCT Filed: Feb. 27, 2024
(86) PCT No.: PCT/KR2024/002516

§ 371 Date: Jul. 18, 2025

(30) Foreign Application Priority Data
Feb. 28, 2023 (KR) 10-2023-0027322

(51) Int. Cl. C12Q 1/6823; C12Q 1/6825; C12Q 1/6853

(52) U.S. Cl. CPC C12Q 1/6823; C12Q 1/6825; C12Q 1/6853

(57) ABSTRACT

The present disclosure relates to detection of target nucleic acid by LPHO-assisted PTO cleavage and extension (L-PTOCE) assay. The method and composition according to the present disclosure ensures detection of one or more target nucleic acids with improved accuracy and convenience.

### TECHNICAL FIELD

The present disclosure relates to detection of a target nucleic acid by LPHO-assisted PTO cleavage and extension (L-PTOCE) assay.

### BACKGROUND ART

For detection of target nucleic acids, real-time detection methods capable of detecting target nucleic acids with monitoring target amplification in a real-time manner are widely used. The real-time detection methods generally use labeled probes or primers specifically hybridized with target nucleic acids.

Examples of methods using hybridization between labeled probes and target nucleic acids include Molecular beacon method using dual-labeled probes with hairpin structure (Tyagi et al., Nature Biotechnology v.14 Mar. 1996), HyBeacon method (French D J et al., Mol. Cell Probes, 15(6):363-374 (2001)), Hybridization probe method using two probes, each labeled as donor and acceptor (Bernard et al., 147-148 Clin. Chem. 2000; 46) and Lux method using single-labeled oligonucleotides (U.S. Pat. No. 7,537,886). TaqMan method (U.S. Pat. Nos. 5,210,015 and 5,538,848) using cleavage of dual-labeled probes by 5′-nuclease activity of DNA polymerase is also widely used in the art.

Examples of methods using labeled primers include Sunrise primer method (Nazarenko et al., 2516-2521 Nucleic Acids Research, 1997, v.25 no. 12, and U.S. Pat. No. 6,117,635), Scorpion primer method (Whitcombe et al., 804-807, Nature Biotechnology v.17 Aug. 1999 and U.S. Pat. No. 6,326,145), and TSG primer method (WO 2011/078441).

Since the above-described conventional real-time detection technologies can only detect a single target nucleic acid per a label, the number of target nucleic acids that can be simultaneously detected in a single reaction is limited by the number of labels that can be used (e.g., 5 or less).

Although melting analysis may be used to detect multiple target nucleic acids with the use of a single label, it has disadvantages that a longer performance time is required compared to the real-time detection technologies, and the design of probes with different Tm values becomes increasingly challenging as the number of target nucleic acids increases.

As such, conventional real-time detection technologies or melting analysis is limited when detecting multiple target nucleic acids.

Therefore, there is a need for a real-time detection method that can simultaneously detect a plurality of target nucleic acids in one reaction, despite using a limited number of labels.

Throughout this application, various patents and publications are referenced and citations are provided in parentheses. The disclosure of these patents and publications in their entities are hereby incorporated by references into this application in order to more fully describe this invention and the state of the art to which this invention pertains.

### DISCLOSURE OF INVENTION

### Technical Problem

The present inventors have endeavored to develop a method for detecting multiple target nucleic acids in real-time using a single type of label. As a result, we have established a novel protocol for detection of target nucleic acids, which involves probe hybridization, enzymatic reaction such as 5′ nucleolytic cleavage and extension, and detection of extended duplexes using Labeled Portion Hybridizing Oligonucleotide (LPHO). The present protocol ensures detection of one or more target nucleic acids with improved accuracy and convenience.

Therefore, it is an object of the present disclosure to provide a method for detecting a target nucleic acid in a sample by LPHO-assisted PTO Cleavage and Extension (L-PTOCE) assay.

It is another object of the present disclosure to provide a composition for detecting a target nucleic acid in a sample.

It is another object of the present disclosure to provide a method for detecting n target nucleic acids in a sample.

It is another object of the present disclosure to provide a method for detecting a target nucleic acid in a sample using a Labeled Portion Hybridizing Oligonucleotide (LPHO).

Other objects and advantages of the present invention will become apparent from the detailed description to follow taken in conjugation with the appended claims and drawings.

### Solution to Problem

According to one aspect of the present disclosure, provided is a method for detecting a target nucleic acid in a sample by LPHO-assisted PTO Cleavage and Extension (L-PTOCE) assay, comprising:
(a) hybridizing a primer and a Probing and Tagging Oligonucleotide (PTO) with the target nucleic acid;
wherein the primer comprises a hybridizing nucleotide sequence with a first region of the target nucleic acid,
wherein the PTO comprises in a 5′ to 3′ direction: (i) a 5′-tagging portion, and (ii) a 3′-targeting portion,
wherein the 3′-targeting portion comprises a hybridizing nucleotide sequence with a second region of the target nucleic acid, and the 5′-tagging portion comprises a nucleotide sequence not hybridized with the target nucleic acid when the 3′-targeting portion is hybridized with the second region of the target nucleic acid,
wherein the primer is located upstream of the PTO;
(b) contacting the resultant of the step (a) to a DNA polymerase having 5′ nuclease activity under conditions for cleavage of the PTO;
wherein the primer is extended to induce cleavage of the PTO by the DNA polymerase having 5′ nuclease activity such that the cleavage releases a fragment comprising the 5′-tagging-portion of the PTO;
(c) hybridizing the fragment released from the PTO with a Capturing and Templating Oligonucleotide (CTO);
wherein the CTO comprises in a 3′ to 5′ direction: (i) a capturing portion comprising a hybridizing nucleotide sequence with the 5′-tagging portion of the PTO, and (ii) a templating portion comprising a non-hybridizing nucleotide sequence with the 5′-tagging portion and the 3′-targeting portion of the PTO,
wherein the CTO has a reporter molecule and a quencher molecule defining a labeled portion,
wherein the fragment is hybridized with the capturing portion of the CTO;
(d) performing an extension reaction using the resultant of the step (c) and the DNA polymerase having 5′ nuclease activity in the presence of a Labeled Portion Hybridizing Oligonucleotide (LPHO);
wherein the LPHO comprises a hybridizing nucleotide sequence with the labeled portion of the CTO,
wherein when the target nucleic acid is present in the sample, the fragment hybridized with the capturing portion of the CTO is extended to generate an extended strand complementary to the CTO, thereby generating an extended duplex between the extended strand and the CTO, wherein the generation of the extended duplex prevents the formation of a CTO/LPHO hybrid between the labeled portion of the CTO and the LPHO,
wherein when the target nucleic acid is not present in the sample, the extended strand is not generated, and instead the CTO/LPHO hybrid is formed,
wherein the extended duplex has a melting temperature (Tm) that is different from a Tm of the CTO/LPHO hybrid; and
(e) detecting the presence of the extended duplex;
wherein the extended duplex is detected by measuring a signal provided from the extended duplex,
wherein the measurement is performed at a temperature at which the signal intensity provided from the extended duplex is different from a signal intensity provided from the CTO/LPHO hybrid, and
wherein the presence of the extended duplex indicates the presence of the target nucleic acid.

In an embodiment, the extended duplex is generated by (i) extending the fragment hybridized to the capturing portion of the CTO prior to the hybridization of the labeled portion of the CTO and the LPHO, (ii) extending the fragment hybridized to the capturing portion of the CTO upon the hybridization between the labeled portion of the CTO and the LPHO, thereby cleaving the LPHO, or (iii) both (i) and (ii).

In an embodiment, the generation of the extended duplex prevents the formation of the CTO/LPHO hybrid, via preference for hybridization between the extended strand and the CTO over hybridization between the labeled portion of the CTO and the LPHO.

In an embodiment, the generation of the extended duplex prevents the formation of the CTO/LPHO hybrid, via cleavage of the LPHO during the extension of step (d).

In an embodiment, when the CTO is unhybridized with the extended strand or the LPHO, the reporter molecule and the quencher molecule on the CTO are in close proximity to each other, thereby causing the quencher molecule to quench a signal from the reporter molecule.

In an embodiment, when the CTO is hybridized with the extended strand or the LPHO, the reporter molecule and the quencher molecule on the CTO are separated, thereby causing the quencher molecule to unquench a signal from the reporter molecule.

In an embodiment, (i) both the reporter molecule and the quencher molecule are linked to the capturing portion of the CTO, (ii) both the reporter molecule and the quencher molecule are linked to the templating portion of the CTO, or (iii) one of the reporter molecule and the quencher molecule is linked to the capturing portion of the CTO and the other is linked to the templating portion of the CTO.

In an embodiment, the LPHO is hybridized with a complete or a partial sequence of the labeled portion of the CTO, and the reporter molecule and the quencher molecule on the CTO are separated, thereby causing the quencher molecule to unquench a signal from the reporter molecule.

In an embodiment, the Tm of the extended duplex is at least 3° C. higher than the Tm of the CTO/LPHO hybrid.

In an embodiment, the Tm of the extended duplex is adjustable by (i) a sequence and/or length of the fragment, (ii) a sequence and/or length of the CTO or (iii) the sequence and/or length of the fragment and the sequence and/or length of the CTO, and the Tm of the CTO/LPHO hybrid is adjustable by a sequence and/or length of the LPHO.

In an embodiment, the LPHO comprises a nucleotide sequence which competes with the fragment for hybridization with the CTO.

In an embodiment, the LPHO is not cleaved by the fragment or its extension product.

In an embodiment, the LPHO comprises a nucleotide sequence which does not compete with the fragment for hybridization with the CTO.

In an embodiment, the LPHO is cleaved by the fragment or its extension product.

In an embodiment, the temperature for measurement depends on the Tm of the extended duplex and the Tm of the CTO/LPHO hybrid.

In an embodiment, the method is performed in the presence of a plurality of the PTOs, a plurality of the CTOs, and a plurality of the LPHOs, and the steps (a)-(e) are repeated with denaturation between repeating cycles.

In an embodiment, the temperature for measurement allows both (i) at least one of the extended duplexes to remain its double-stranded state and (ii) at least one of the CTO/LPHO hybrids to dissociate into a single-stranded state.

According to another aspect of the present disclosure, provided is a composition for detecting a target nucleic acid in a sample, comprising:
(a) a primer;
wherein the primer comprises a hybridizing nucleotide sequence with a first region of the target nucleic acid,
(b) a Probing and Tagging Oligonucleotide (PTO);
wherein the PTO comprises in a 5′ to 3′ direction: (i) a 5′-tagging portion, and (ii) a 3′-targeting portion,
wherein the 3′-targeting portion comprises a hybridizing nucleotide sequence with a second region of the target nucleic acid, and the 5′-tagging portion comprises a nucleotide sequence not hybridized with the target nucleic acid when the 3′-targeting portion is hybridized with the second region of the target nucleic acid,
wherein the primer is located upstream of the PTO,
wherein the primer is extended to induce cleavage of the PTO by a DNA polymerase having 5′ nuclease activity such that the cleavage releases a fragment comprising the 5′-tagging-portion of the PTO;
(c) a Capturing and Templating Oligonucleotide (CTO);
wherein the CTO comprises in a 3′ to 5′ direction: (i) a capturing portion comprising a hybridizing nucleotide sequence with the 5′-tagging portion of the PTO, and (ii) a templating portion comprising a non-hybridizing nucleotide sequence with the 5′-tagging portion and the 3′-targeting portion of the PTO,
wherein the CTO has a reporter molecule and a quencher molecule defining a labeled portion,
wherein the fragment is hybridized with the capturing portion of the CTO; and
(d) a Labeled Portion Hybridizing Oligonucleotide (LPHO);
wherein the LPHO comprises a hybridizing nucleotide sequence with the labeled portion of the CTO,
wherein when the target nucleic acid is present in the sample, the fragment hybridized with the capturing portion of the CTO is extended to generate an extended strand complementary to the CTO, thereby generating an extended duplex between the extended strand and the CTO, wherein the generation of the extended duplex prevents the formation of a CTO/LPHO hybrid between the labeled portion of the CTO and the LPHO,
wherein when the target nucleic acid is not present in the sample, the extended strand is not generated, and instead the CTO/LPHO hybrid is formed,
wherein the extended duplex has a melting temperature (Tm) that is different from a Tm of the CTO/LPHO hybrid.

In an embodiment, when the CTO is unhybridized with the extended strand or the LPHO, the reporter molecule and the quencher molecule on the CTO are in close proximity to each other, thereby causing the quencher molecule to quench a signal from the reporter molecule.

In an embodiment, when the CTO is hybridized with the extended strand or the LPHO, the reporter molecule and the quencher molecule on the CTO are separated, thereby causing the quencher molecule to unquench a signal from the reporter molecule.

In an embodiment, (i) both the reporter molecule and the quencher molecule are linked to the capturing portion of the CTO, (ii) both the reporter molecule and the quencher molecule are linked to the templating portion of the CTO, or (iii) one of the reporter molecule and the quencher molecule is linked to the capturing portion of the CTO and the other is linked to the templating portion of the CTO.

In an embodiment, the LPHO is hybridized with a complete or a partial sequence of the labeled portion of the CTO, and the reporter molecule and the quencher molecule on the CTO are separated, thereby causing the quencher molecule to unquench a signal from the reporter molecule.

In an embodiment, the LPHO comprises a nucleotide sequence which competes with the fragment for hybridization with the CTO.

In an embodiment, the LPHO comprises a nucleotide sequence which does not compete with the fragment for hybridization with the CTO.

In an embodiment, the composition provides a signal dependent on the presence of the target nucleic acid.

In an embodiment, the signal dependent on the presence of the target nucleic acid is a signal provided from the extended duplex.

In an embodiment, the composition has a signal-changing temperature range (SChTR) in which the signal changes depending on the presence of the target nucleic acid, and two signal-constant temperature ranges (SCoTRs) in which the signal is constant even in the presence of the target nucleic acid,

In an embodiment, the signal-changing temperature range is higher than a first signal-constant temperature range of the two signal-constant temperature ranges, and lower than a second signal-constant temperature range of the two signal-constant temperature ranges.

In an embodiment, the extended duplex remains its double-stranded state and the CTO/LPHO hybrid dissociates into a single-stranded state at temperatures within the signal-changing temperature range in the presence of the target nucleic acid.

According to another aspect of the present disclosure, provided is a method for detecting n target nucleic acids in a sample, comprising:
(a) detecting signals at n detection temperatures, while incubating n compositions for detecting the n target nucleic acids with a sample suspected of containing at least one of the n target nucleic acids in a reaction vessel;
wherein n is an integer of 2 or more,
wherein the incubating comprises a plurality of reaction cycles and the detection of signals is carried out in at least one of the plurality of reaction cycles,
wherein each of the n compositions for detecting the n target nucleic acids provides a signal change at a corresponding detection temperature among the n detection temperatures in the presence of a corresponding target nucleic acid, the signal change indicating the presence of a corresponding target nucleic acid,
wherein a composition for detecting an i^{th} target nucleic acid among the n compositions for detecting the n target nucleic acids provides a signal change at an i^{th} detection temperature among the n detection temperatures and provides a constant signal at the other detection temperatures in the presence of the i^{th} target nucleic acid, the signal change indicating the presence of the i^{th} target nucleic acid,
wherein i represents an integer from 1 to n, and the i^{th} detection temperature is lower than a (i+1)^{th} detection temperature,
wherein within the temperature range covering all the n detection temperatures, the composition for detecting the i^{th} target nucleic acid has a signal-changing temperature range (SChTR) in which the signal changes depending on the presence of the i^{th} target nucleic acid, and one or two signal-constant temperature ranges (SCoTRs) in which the signal is constant even in the presence of the i^{th} target nucleic acid,
wherein the composition for detecting the i^{th} target nucleic acid is any one of:
(i) an Under-Signal-Change-type (UnderSC-type) composition having a melting characteristic that the signal-changing temperature range is lower than the signal-constant temperature range,
(ii) an Inter-Signal-Change-type (InterSC-type) composition having a melting characteristic that the signal-changing temperature range is higher than one of two signal-constant temperature ranges, and lower than the other of the two signal-constant temperature ranges, and
(iii) an Over-Signal-Change-type (OverSC-type) composition having a melting characteristic that the signal-changing temperature range is higher than the signal-constant temperature range, and
wherein at least one of the n compositions for detecting n target nucleic acids is (ii) an InterSC-type composition which generates the signal according to the L-PTOCE assay as described above, and
(b) determining the presence of the n target nucleic acids from the signals detected in the step (a), wherein the presence of the i^{th} target nucleic acid is determined by the signal change detected at the i^{th} detection temperature.

In an embodiment, the i^{th} detection temperature is selected within the signal-changing temperature range of the composition for detecting the i^{th} target nucleic acid, wherein the i^{th} detection temperature is not comprised in the signal-changing temperature ranges of the compositions for detecting the other target nucleic acids.

In an embodiment, the signal-changing temperature range of the composition for detecting the i^{th} target nucleic acid overlaps partially with the signal-changing temperature range of a composition for detecting a target nucleic acid having an adjacent detection temperature, and does not overlap with the signal-changing temperature range of a composition for detecting a target nucleic acid having a detection temperature that is not adjacent thereto.

In an embodiment, when n is 2, the composition for detecting the first target nucleic acid is an UnderSC-type or InterSC-type composition, and the composition for detecting the second target nucleic acid is an InterSC-type or OverSC-type composition.

In an embodiment, when n is 3 or more, the composition for detecting the first target nucleic acid is an UnderSC-type or InterSC-type composition, the composition for detecting the n^{th} target nucleic acid is an InterSC-type or OverSC-type composition, and each of compositions for detecting target nucleic acids other than the first target nucleic acid and the n^{th} target nucleic acid is an InterSC-type composition.

In an embodiment, the composition for detecting the i^{th} target nucleic acid comprises a label that provides a signal dependent on the presence of the i^{th} target nucleic acid.

In an embodiment, the label is linked to an oligonucleotide or is incorporated into an oligonucleotide during the incubating.

In an embodiment, the composition for detecting the i^{th} target nucleic acid provides a duplex providing a signal change.

In an embodiment, the duplex providing the signal change has initially been included in the composition for detecting the i^{th} target nucleic acid.

In an embodiment, the duplex providing the signal change is generated by hy-bridization between a labeled oligonucleotide and an oligonucleotide hybridizable with the labeled oligonucleotide.

In an embodiment, the duplex providing the signal change is generated during in-cubating.

In an embodiment, the duplex providing the signal change is generated by hy-bridization between a labeled oligonucleotide and the corresponding target nucleic acid.

In an embodiment, the duplex providing the signal change is generated by a cleavage reaction dependent on the presence of the corresponding target nucleic acid.

In an embodiment, the duplex providing the signal change comprises a label.

In an embodiment, the composition for detecting the i^{th} target nucleic acid provides a duplex providing a signal change, and the signal-changing temperature range of the composition for detecting the i^{th} target nucleic acid varies depending on the length and/or sequence of the duplex.

In an embodiment, the detection of signals is carried out in at least two of the plurality of reaction cycles.

In an embodiment, the signal change is measured using the signals detected in the at least two of the plurality of reaction cycles.

In an embodiment, the signal change at the i^{th} detection temperature is measured using a signal detected in at least one of the plurality of reaction cycles and a reference signal value.

In an embodiment, the reference signal value is obtained from a reaction in the absence of the i^{th} target nucleic acid.

In an embodiment, the detection of a signal at each of the n detection temperatures is carried out using a single type of detector.

In an embodiment, the signals detected at the n detection temperatures are not differ-entiated from each other by the single type of detector.

In an embodiment, the incubating comprises a nucleic acid amplification reaction.

According to another aspect of the present disclosure, provided is a method for detecting a target nucleic acid in a sample using a Labeled Portion Hybridizing Oligonucleotide (LPHO), comprising:
(a) providing a fragment produced by an enzymatic cleavage reaction of an oligonu-cleotide depending on the presence of the target nucleic acid in the sample;
(b) hybridizing the fragment with a Capturing and Templating Oligonucleotide (CTO);
wherein the CTO comprises in a 3′ to 5′ direction: (i) a capturing portion comprising a hybridizing nucleotide sequence with the fragment, and (ii) a templating portion comprising a non-hybridizing nucleotide sequence with the fragment,
wherein the CTO has a reporter molecule and a quencher molecule defining a labeled portion,
wherein the fragment is hybridized with the capturing portion of the CTO;
(c) performing an extension reaction using the resultant of the step (b) and a DNA polymerase having 5′ nuclease activity in the presence of the LPHO;
wherein the LPHO comprises a hybridizing nucleotide sequence with the labeled portion of the CTO,
wherein when the target nucleic acid is present in the sample, the fragment hybridized with the capturing portion of the CTO is extended to generate an extended strand complementary to the CTO, thereby generating an extended duplex between the extended strand and the CTO, wherein the generation of the extended duplex prevents the formation of a CTO/LPHO hybrid between the labeled portion of the CTO and the LPHO,
wherein when the target nucleic acid is not present in the sample, the extended strand is not generated, and instead the CTO/LPHO hybrid is formed,
wherein the extended duplex has a melting temperature (Tm) that is different from a Tm of the CTO/LPHO hybrid; and
(d) detecting the presence of the extended duplex;
wherein the extended duplex is detected by measuring a signal provided from the extended duplex,
wherein the measurement is performed at a temperature at which the signal intensity from the extended duplex is different from a signal intensity from the CTO/LPHO hybrid, and
wherein the presence of the extended duplex indicates the presence of the target nucleic acid.

### Advantageous Effects of Invention

The features and advantages of the present disclosure will be summarized as follows:
(a) The first feature of the present disclosure is to utilize (i) a PTO that hybridizes with the target nucleic acid, (ii) a CTO that has a reporter molecule and a quencher molecule defining a labeled portion and is capable of generating an extended duplex in the presence of the target nucleic acid, and (iii) a LPHO that comprises a hybridizing nucleotide sequence with the labeled portion of the CTO, for the detection of the target nucleic acid. In particular, the reporter and quencher molecules on the CTO are in close proximity to each other before the CTO is hybridized with another oligonu-cleotide (such as LPHO or extended strand), and the quencher molecule quenches the signal from the reporter molecule. However, upon hybridization of the CTO with another oligonucleotide (such as LPHO or extended strand), the reporter and quencher molecules on the CTO are separated, causing the quencher molecule to unquench a signal from the reporter molecule.
(b) The second feature of the present disclosure is that the CTO/LPHO hybrid has a Tm adjustable by a sequence and/or length of the LPHO, and the extended duplex has a Tm adjustable by (i) a sequence and/or length of the fragment, (ii) a sequence and/or length of the CTO or (iii) the sequence and/or length of the fragment and the sequence and/or length of the CTO. By using this feature, the Tm of the CTO/LPHO hybrid and the Tm of the extended duplex can be pre-determined to be different from each other. These two different Tm values allow the composition for detecting a target nucleic acid according to the present disclosure to have a signal-changing temperature range in which the signal changes depending on the presence of the target nucleic acid, and two signal-constant temperature ranges in which the signal is constant even in the presence of the target nucleic acid.
(c) The composition according to the present disclosure, as an InterSC-type composition, enables the detection of one or more target nucleic acids using a single type of label in a single reaction vessel. Specifically, when using a plurality of compositions for detecting a plurality of target nucleic acids, a plurality of target nucleic acids can be detected in real-time using a single type of label by adjusting the signal-changing temperature range of each of the compositions for detecting a plurality of target nucleic acids, in particular, by adjusting signal-changing temperature ranges not to overlap with each other. Moreover, the method of the present disclosure has the advantage of dramatically reducing its analysis time compared to conventional melting analysis after target amplification to detect multiple target nucleic acids using a single type of label.
(d) Furthermore, the method according to the present disclosure is effective in detecting nucleotide variations with a low occurrence of false-positives.

### BRIEF DESCRIPTION OF DRAWINGS

FIG. 1 illustrates the L-PTOCE assay of the present disclosure.

FIG. 2A shows various embodiments where the LPHO to be hybridized with the labeled portion of the CTO completely (ii, iii, iv, or vi) or partially (i or v) overlaps with the PTO fragment to be hybridized with the capturing portion of the CTO.

FIG. 2B shows various embodiments where the LPHO to be hybridized with the labeled portion of the CTO does not overlap with the PTO fragment to be hybridized with the capturing portion of the CTO.

FIG. 3 show, in the absence of the target nucleic acid or before reaction between the target nucleic acid and the L-PTOCE composition, conformational changes of the CTO and the LPHO (i.e., the CTO/LPHO hybrid) at (i) the first signal-constant temperature range, (ii) the signal-changing temperature range, and (iii) the second signal-constant temperature range;

FIG. 4 show, after reaction of target nucleic acid and the L-PTOCE composition, con-formational changes of the CTO and the extended strand (i.e., extended duplex) at (i) the first signal-constant temperature range, (ii) the signal-changing temperature range, and (iii) the second signal-constant temperature range.

FIG. 5A shows the amount (or abundance) ratios of the CTO/LPHO hybrid and the extended duplex in the initial, intermediate, and late cycles of steps (a)-(e) of the L-PTOCE assay, along with their melt curves.

FIG. 5B represents a merged plot for three melt curves in FIG. 5A.

FIG. 6 shows real-time PCR results for Combination 1 of Example 1.

FIG. 7 shows real-time PCR results for Combination 2 of Example 1.

FIG. 8 shows real-time PCR results for Combination 3 of Example 1.

FIG. 9 shows real-time PCR results for Combination 4 of Example 1.

FIG. 10 shows real-time RT-PCR results for nucleotide variation detection in Example 2.

FIG. 11 schematically shows the signal generation mechanisms of two compositions, an UnderSC-type composition and an InterSC-type composition, used in Example 3.

FIG. 12 shows multiplex real-time PCR results in Example 3.

### BEST MODE FOR CARRYING OUT THE INVENTION

The present inventors have endeavored to develop a method for detecting multiple target nucleic acids in real-time using a single type of label. As a result, we have es-tablished a novel protocol for detection of target nucleic acids, which involves probe hybridization, enzymatic reaction such as 5′ nucleolytic cleavage and extension, and detection of extended duplexes using Labeled Portion Hybridizing Oligonucleotide (LPHO). The present protocol ensures detection of one or more target nucleic acid with improved accuracy and convenience.

### I. Method for Detecting Target Nucleic Acid by L-PTOCE Assay

In a first aspect of the present disclosure, there is provided a method for detecting a target nucleic acid in a sample by LPHO-assisted PTO Cleavage and Extension (L-PTOCE) assay, comprising:
(a) hybridizing a primer and a Probing and Tagging Oligonucleotide (PTO) with the target nucleic acid;
wherein the primer comprises a hybridizing nucleotide sequence with a first region of the target nucleic acid,
wherein the PTO comprises in a 5′ to 3′ direction: (i) a 5′-tagging portion, and (ii) a 3′-targeting portion,
wherein the 3′-targeting portion comprises a hybridizing nucleotide sequence with a second region of the target nucleic acid, and the 5′-tagging portion comprises a nucleotide sequence not hybridized with the target nucleic acid when the 3′-targeting portion is hybridized with the second region of the target nucleic acid,
wherein the primer is located upstream of the PTO;
(b) contacting the resultant of the step (a) to a DNA polymerase having 5′ nuclease activity under conditions for cleavage of the PTO;
wherein the primer is extended to induce cleavage of the PTO by the DNA polymerase having 5′ nuclease activity such that the cleavage releases a fragment comprising the 5′-tagging-portion of the PTO;
(c) hybridizing the fragment released from the PTO with a Capturing and Templating Oligonucleotide (CTO);
wherein the CTO comprises in a 3′ to 5′ direction: (i) a capturing portion comprising a hybridizing nucleotide sequence with the 5′-tagging portion of the PTO, and (ii) a templating portion comprising a non-hybridizing nucleotide sequence with the 5′-tagging portion and the 3′-targeting portion of the PTO,
wherein the CTO has a reporter molecule and a quencher molecule defining a labeled portion,
wherein the fragment is hybridized with the capturing portion of the CTO;
(d) performing an extension reaction using the resultant of the step (c) and the DNA polymerase having 5′ nuclease activity in the presence of a Labeled Portion Hybridizing Oligonucleotide (LPHO);
wherein the LPHO comprises a hybridizing nucleotide sequence with the labeled portion of the CTO,
wherein when the target nucleic acid is present in the sample, the fragment hybridized with the capturing portion of the CTO is extended to generate an extended strand complementary to the CTO, thereby generating an extended duplex between the extended strand and the CTO, wherein the generation of the extended duplex prevents the formation of a CTO/LPHO hybrid between the labeled portion of the CTO and the LPHO,
wherein when the target nucleic acid is not present in the sample, the extended strand is not generated, and instead the CTO/LPHO hybrid is formed,
wherein the extended duplex has a melting temperature (Tm) that is different from a Tm of the CTO/LPHO hybrid; and
(e) detecting the presence of the extended duplex;
wherein the extended duplex is detected by measuring a signal provided from the extended duplex,
wherein the measurement is performed at a temperature at which the signal intensity provided from the extended duplex is different from a signal intensity provided from the CTO/LPHO hybrid, and
wherein the presence of the extended duplex indicates the presence of the target nucleic acid.

Such denotations as “first,” “second,” “A,” “B,” “(a),” “(b),” “(i),” and “(ii)” may be used in describing the components of the present disclosure. These denotations are provided merely to distinguish a component from another, and the essence of the components is not limited by the denotations in light of order or sequence.

The method according to the present disclosure employs successive events occurred by probe hybridization, i.e., cleavage and extension of PTO; generation of extended duplex; and detection of the extended duplex using LPHO, which is termed “LPHO-assisted PTO Cleavage and Extension (L-PTOCE)” assay.

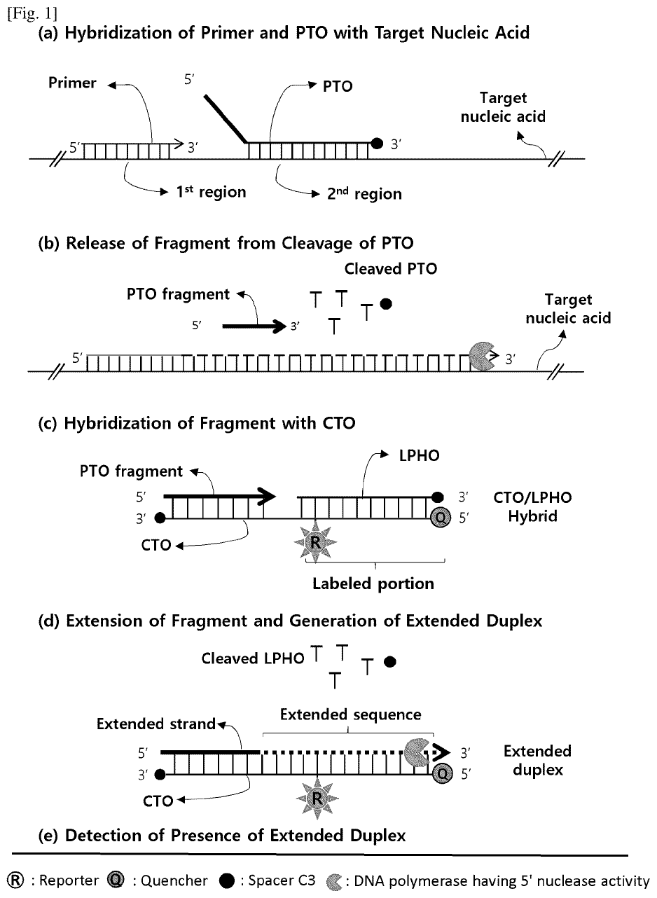

The L-PTOCE assay of the present disclosure is schematically illustrated in FIG. 1. The L-PTOCE assay will be described in more detail as follows:

Step (a): Hybridization of Primer and PTO with Target Nucleic Acid

According to the present disclosure, a target nucleic acid in a sample is hybridized with a primer and a PTO (Probing and Tagging Oligonucleotide).

The term “target nucleic acid”, “target nucleic acid sequence”, or “target sequence” as used herein refers to a nucleic acid sequence to be detected or quantified. The target nucleic acid sequence includes a single-strand as well as a double-strand. The target nucleic acid sequence includes, not only a sequence newly generated in a reaction, but also a sequence initially present in a nucleic acid sample.

The target nucleic acid includes any DNA (gDNA and cDNA) and RNA molecules, and their hybrids (chimeric nucleic acids). The sequence may be in a double-stranded or single-stranded form.

Target nucleic acids include any naturally occurring procaryotic, eukaryotic (for example, protozoans and parasites, fungi, yeast, higher plants, lower animals, and higher animals, including mammals and humans) or viral (for example, Herpes virus, HIV, influenza virus, Epstein-Barr virus, hepatitis virus, polio virus, etc.) or viroid nucleic acid. In addition, the nucleic acid molecule may be any nucleic acid molecule which is produced or can be produced by recombination, or any nucleic acid molecule which is or can be chemically synthesized. As such, the nucleic acid sequence may or may not be found in nature. The target nucleic acid sequence may be known or unknown.

As used herein, the term “sample” refers to cells, tissues or fluid from a biological source, or any other medium that may be proven to be useful in the present invention, and includes virus, bacteria, tissues, cells, blood, serum, plasma, lymph, milk, urine, feces, intraocular fluid, saliva, semen, brain extract, spinal fluid, appendix, spleen and tonsil tissue extracts, amniotic fluid, ascites, and non-biological samples (e.g., food and water). In addition, the sample includes naturally occurring nucleic acid molecule isolated from a biological source, and synthesized nucleic acid molecules.

As used herein, the term “primer” refers to an oligonucleotide, which is capable of acting as a point of initiation of synthesis when placed under conditions in which synthesis of primer extension product which is complementary to a target nucleic acid (template) is induced, i.e., in the presence of nucleotides and an agent for poly-merization, such as DNA polymerase, and at suitable temperatures and pH. The primer should be sufficiently long to prime the synthesis of extension product in the presence of the agent for polymerization. A suitable length of the primers will depend on many factors, including temperature, application, and source of primer.

The term “probe” as used herein refers to a single-stranded nucleic acid molecule comprising a hybridizing portion(s) to a target nucleic acid sequence. Herein, PTO serves as a probe.

In particular, the probe and primer are single-stranded deoxyribonucleotide molecules. The probes or primers used in this invention may be comprised of naturally occurring dNMP (i.e., dAMP, dGM, dCMP and dTMP), modified nucleotide, or non-natural nucleotide. The probes or primers may also include ribonucleotides.

The term “annealing” or “priming” as used herein refers to the apposition of an oligodeoxynucleotide or nucleic acid to a template nucleic acid, whereby the ap-position enables the polymerase to polymerize nucleotides into a nucleic acid molecule which is complementary to the template nucleic acid or a portion thereof.

The term “hybridize”, “hybridizing” or “hybridization” as used herein refers to the formation of double strands by noncovalent association between two complementary single-stranded polynucleotides under certain hybridization conditions or stringent conditions.

The hybridization may occur between two nucleic acid strands perfectly matched or substantially matched with some mismatches (e.g., 1-4 mismatches). The comple-mentarity for hybridization may depend on hybridization conditions, particularly temperature.

The hybridization of a target nucleic acid with the primer and the PTO may be carried out under suitable hybridization conditions routinely determined by opti-mization procedures. Conditions such as temperature, concentration of components, hybridization and washing times, buffer components, and their pH and ionic strength may be varied depending on various factors, including the length and GC content of oligonucleotide (primer and PTO) and the target nucleotide sequence. For instance, when a relatively short oligonucleotide is used, it is preferable that low stringent conditions are adopted. The detailed conditions for hybridization can be found in Joseph Sambrook, et al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999).

There is no intended distinction between the terms “annealing” and “hybridizing”, and these terms will be used interchangeably.

In one embodiment, the primer used in the present disclosure comprises a hybridizing nucleotide sequence with a first region of the target nucleic acid.

In particular, the expression herein that one oligonucleotide (e.g., primer or PTO) “comprises a hybridizing nucleotide sequence” to another oligonucleotide (e.g., target nucleic acid) means that all or a portion of one oligonucleotide has a complementary nucleotide sequence necessary for hybridization with all or a portion of another oligonucleotide. Further, when referring to hybridization of a portion of one oligonu-cleotide to another oligonucleotide, the portion of one oligonucleotide can be regarded as an individual oligonucleotide.

The term “complementary” is used herein to mean that primers or probes are suf-ficiently complementary to hybridize selectively to a target nucleic acid under the designated annealing conditions or stringent conditions, encompassing the terms “sub-stantially complementary” and “perfectly complementary”, particularly perfectly com-plementary.

In contrast, the term “non-complementary” is used herein to mean that primers or probes are sufficiently non-complementary not to hybridize selectively to a target nucleic acid under the designated annealing conditions or stringent conditions, en-compassing the terms “substantially non-complementary” and “perfectly non-complementary”, particularly perfectly non-complementary.

As used herein, the term “Probing and Tagging Oligonucleotide (PTO)” means an oligonucleotide comprising in a 5′ to 3′ direction: (i) a 5′-tagging portion, and (ii) a 3′-targeting portion, and wherein the 3′-targeting portion comprises a hybridizing nucleotide sequence with a second region of the target nucleic acid, and the 5′-tagging portion comprises a nucleotide sequence not hybridized with the target nucleic acid when the 3′-targeting portion is hybridized with the second region of the target nucleic acid.

In one embodiment, the PTO comprises in a 5′ to 3′ direction: (i) a 5′-tagging portion comprising a non-hybridizing nucleotide sequence to the target nucleic acid and (ii) a 3′-targeting portion comprising a hybridizing nucleotide sequence with a second region of the target nucleic acid; wherein the 5′-tagging portion of PTO is not hybridized with the target nucleic acid but the 3′-targeting portion of PTO is hybridized with the target nucleic acid. In other words, the PTO comprises two portions as follows: (i) a 3′-targeting portion serving as a probe and (ii) a 5′-tagging portion which is nucle-olytically released from the PTO after hybridization with the target nucleic acid. The 5′-tagging portion and the 3′-targeting portion in the PTO have to be positioned in 5′ to 3′ order.

The expression herein that one oligonucleotide (e.g., the 5′-tagging portion of the PTO) “comprises a non-hybridizing nucleotide sequence” to another oligonucleotide (e.g., the target nucleic acid) means that one oligonucleotide has a non-complementary nucleotide sequence necessary for non-hybridization with another oligonucleotide.

In one embodiment, the hybridization in the step (a) is performed under stringent conditions such that the 3′-targeting portion of the PTO is hybridized with the second region of the target nucleic acid and the 5′-tagging portion of the PTO is not hybridized with the target nucleic acid.

The PTO does not require any specific lengths. For example, the length of the PTO may be 15-150 nucleotides, 15-100 nucleotides, 15-80 nucleotides, 15-60 nucleotides, 15-40 nucleotides, 20-150 nucleotides, 20-100 nucleotides, 20-80 nucleotides, 20-60 nucleotides, 20-50 nucleotides, 30-150 nucleotides, 30-100 nucleotides, 30-80 nu-cleotides, 30-60 nucleotides, 30-50 nucleotides, 35-100 nucleotides, 35-80 nucleotides, 35-60 nucleotides, or 35-50 nucleotides.

The 3′-targeting portion of the PTO may be in any lengths so long as it is specifically hybridized with target nucleic acid sequences. For example, the 3′-targeting portion of the PTO may be 10-100 nucleotides, 10-80 nucleotides, 10-50 nucleotides, 10-40 nu-cleotides, 10-30 nucleotides, 15-100 nucleotides, 15-80 nucleotides, 15-50 nucleotides, 15-40 nucleotides, 15-30 nucleotides, 20-100 nucleotides, 20-80 nucleotides, 20-50 nu-cleotides, 20-40 nucleotides, or 20-30 nucleotides in length.

The 5′-tagging portion may be in any lengths so long as it is specifically hybridized with the templating portion of the CTO and then extended. For instance, the 5′-tagging portion of the PTO may be 5-50 nucleotides, 5-40 nucleotides, 5-30 nucleotides, 5-20 nucleotides, 10-50 nucleotides, 10-40 nucleotides, 10-30 nucleotides, 10-20 nu-cleotides, 15-50 nucleotides, 15-40 nucleotides, 15-30 nucleotides, or 15-20 nu-cleotides in length.

In one embodiment, the 3′-end of the PTO may have a 3′-OH terminal. In particular, the 3′-end of the PTO is “blocked” to prohibit its extension.

The blocking may be achieved in accordance with conventional methods. For instance, the blocking may be performed by adding to the 3′-hydroxyl group of the last nucleotide a chemical moiety such as biotin, labels, a phosphate group, alkyl group, non-nucleotide linker, phosphorothioate or alkane-diol. Alternatively, the blocking may be carried out by removing the 3′-hydroxyl group of the last nucleotide or using a nucleotide with no 3′-hydroxyl group such as dideoxynucleotide.

Alternatively, the PTO may be designed to have a hairpin structure.

The non-hybridization between the 5′-tagging portion of the PTO and the target nucleic acid refers to non-formation of a stable double-strand between them under certain hybridization conditions. In one embodiment, the 5′-tagging portion of the PTO not involved in the hybridization with the target nucleic acid forms a single-strand.

The primer used in the present disclosure refers to an upstream primer located upstream of the PTO. When the target nucleic acid is double stranded, the primer and the PTO are hybridized with one strand of the double-stranded target nucleic acid, and the PTO is positioned downstream of the primer. The primer is hybridized with a specific portion (i.e., a first region of the target nucleic acid) in the 3′-direction relative to the portion (i.e., a second region of the target nucleic acid) of the target nucleic acid strand to which the PTO is hybridized.

In one embodiment, when a target nucleic acid is double-stranded, one strand of the double-stranded target nucleic acid comprises both a first region and a second region of the target nucleic acid. In particular, one strand of the double-stranded target nucleic acid comprises in 3′ to 5′ direction: (i) a first region to be hybridized with the primer; and (ii) a second region to be hybridized with PTO.

In one embodiment, the method is performed in the presence of an additional primer. The additional primer generates additionally a target nucleic acid to be hybridized with the PTO, enhancing sensitivity in target detection. The additional primer may also be referred to as a downstream primer.

In one embodiment, when the primer and the additional primer are used, a template-dependent nucleic acid polymerase is additionally employed for extension of the primers. The primer and the additional primer may also be referred to as a forward primer and a reverse primer, respectively.

In one embodiment, the primer, the additional primer and/or the 5′-tagging portion of the PTO have a dual priming oligonucleotide (DPO) structure. The oligonucleotides having the DPO structure show significantly improved target specificity compared with conventional primers and probes (see WO 2006/095981; Chun et al., Dual priming oligonucleotide system for the multiplex detection of respiratory viruses and SNP genotyping of CYP2C19 gene, Nucleic Acid Research, 35: 6e40(2007)).

In one embodiment, the 3′-targeting portion of the PTO has a modified dual specificity oligonucleotide (mDSO) structure. The modified dual specificity oligonu-cleotide (mDSO) structure shows significantly improved target specificity compared with conventional probes (see WO 2011/028041).

Step (b): Release of Fragment from Cleavage of PTO

Afterwards, the resultant of the step (a) is contacted to a DNA polymerase having 5′ nuclease activity under conditions for cleavage of the PTO. The primer is extended to induce cleavage of the PTO by the DNA polymerase having 5′ nuclease activity such that the cleavage releases a fragment comprising the 5′-tagging-portion of the PTO.

In one embodiment, extension of the primer induces cleavage of the PTO by the DNA polymerase having 5′ nuclease activity. In particular, the primer is hybridized with a first region of the target nucleic acid, which is positioned away from the PTO, the DNA polymerase having 5′ nuclease activity promotes extension of the primer, and the DNA polymerase having 5′ nuclease activity bound to the extension product cleaves the PTO.

In another embodiment, the primer is hybridized with a first region of the target nucleic acid, which is positioned adjacent to the PTO such that it is sufficient to induce the PTO cleavage by the DNA polymerase having 5′ nuclease activity, and the DNA polymerase having 5′ nuclease activity bound to the primer cleaves the PTO without extension reaction.

Therefore, the induction of the PTO cleavage may be accomplished in two different manners: primer extension-dependent cleavage induction; and (ii) primer extension-independent cleavage induction.

Depending upon the selected cleavage induction manner, the primer may be located relative to the PTO. The primer may be located away from the PTO such that it is sufficient to induce the PTO cleavage in an extension-dependent manner. In other words, the first region and the second region of the target nucleic acid may be apart from each other. Alternatively, the primer may be located adjacent to the PTO such that it is sufficient to induce the PTO cleavage in an extension-independent manner. In other words, the first region and the second region of the target nucleic acid may be in close proximity to each other.

As used herein, the term “adjacent” with referring to positions or locations means that the primer is located adjacent to the 3′-targeting portion of the PTO to form a nick. Also, the term means that the primer is located 1-30 nucleotides, 1-20 nucleotides or 1-15 nucleotides apart from the 3′-targeting portion of the PTO.

As used herein, the term “located away from” with referring to positions or locations includes any positions or locations sufficient to ensure extension reactions.

In one embodiment, the primer is located away from the PTO sufficient to induce the PTO cleavage in an extension-dependent manner.

In one embodiment, the conventional technologies for cleavage reactions by primers may be applied to the present invention, so long as the primer hybridized with the first region of the target nucleic acid induces cleavage of the PTO hybridized with the second region of the target nucleic acid to release a fragment comprising the 5′-tagging portion or a part of the 5′-tagging portion of the PTO. For example, U.S. Pat. Nos. 5,210,015, 5,487,972, 5,691,142, 5,994,069 and 7,381,532 and U.S. Appln. Pub. No. 2008-0241838 may be applied to the present invention.

The phrase “conditions for cleavage of the PTO” as used herein means conditions sufficient to digest the PTO hybridized with the target nucleic acid by an enzyme having 5′ nuclease activity (e.g., DNA polymerase having the 5′ nuclease activity), such as temperature, pH, ionic strength, buffer, length and sequence of oligonu-cleotides and enzymes. For example, when Taq DNA polymerase is used as the enzyme having 5′ nuclease activity, the conditions for cleavage of the PTO include Tris-HCl buffer, KCl, MgCl₂, and temperature.

When the PTO is hybridized with the target nucleic acid, its 3′-targeting portion is involved in the hybridization and its 5′-tagging portion forms a single-strand with no hybridization with the target nucleic acid (see FIG. 1). As such, an oligonucleotide comprising both single-stranded and double-stranded structures may be digested using an enzyme having 5′ nuclease activity by a variety of technologies known to one of skill in the art.

The cleavage sites of the PTO varies depending on the type of primer, hybridization sites of primer, and cleavage conditions (see U.S. Pat. Nos. 5,210,015, 5,487,972, 5,691,142, 5,994,069 and 7,381,532 and U.S. Appln. Pub. No. 2008-0241838).

A multitude of conventional technologies may be employed for the cleavage reaction of the PTO, releasing a fragment comprising the 5′-tagging portion or a part of the 5′-tagging portion.

Briefly, there may be three sites of cleavage in the step (b). The first cleavage site is a junction site between a hybridization portion of the PTO (3′-targeting portion) and a non-hybridization portion (5′-tagging portion). The second cleavage site is a site located several nucleotides in 3′ direction apart from the 3′-end of the 5′-tagging portion of the PTO. The second cleavage site is located at the 5′-end part of the 3′-targeting portion of the PTO. The third cleavage site is a site located several nu-cleotides in 5′ direction apart from the 3′-end of the 5′-tagging to portion of the PTO.

In one embodiment, the initial site for the cleavage of the PTO by the DNA polymerase having 5′ nuclease activity upon extension of the primer is a starting point of the double strand between the PTO and the target nucleic acid or a site 1-3 nu-cleotides apart from the starting point.

In this regard, the phrase “a PTO fragment comprising the 5′-tagging portion of the PTO” in the context of cleavage of the PTO by the DNA polymerase having the 5′ nuclease activity as used herein is used to encompass (i) the 5′-tagging portion, (ii) the 5′-tagging portion and the 5′-end part of the 3′-targeting portion (e.g., the first nucleotide at the 5′-end of the 3′-targeting portion, the first to second nucleotides at the 5′-end of the 3′-targeting portion, the first to third nucleotides at the 5′-end of the 3′-targeting portion, the first to fourth nucleotides at the 5′-end of the 3′-targeting portion, or the first to fifth nucleotides at the 5′-end of the 3′-targeting portion) and (iii) a part of the 5′-tagging portion. The phrase “a fragment comprising the 5′-tagging portion of the PTO” may be abbreviated herein as “PTO fragment” or “fragment”.

The term “part” used in conjunction with the PTO or the CTO such as the part of the 5′-tagging portion of the PTO, the 5′-end part of the 3′-targeting portion of the PTO and the 5′-end part of the capturing portion of the CTO refers to a nucleotide sequence composed of 1-40, 1-30, 1-20, 1-15, 1-10 or 1-5 nucleotides, particularly 1, 2, 3 or 4 nucleotides.

The PTO has a blocker that is resistant to cleavage by enzymes with 5′ nuclease activity, and the blocker is used to control the initial cleavage site and/or subsequent cleavage. For example, the 5′-end part of the 3′-targeting portion of PTO may be blocked with a blocker to induce cleavage at the junction site between the hy-bridization portion (3′-targeting portion) and the non-hybridization portion (5′-tagging portion) of PTO.

In one embodiment, the DNA polymerase having 5′ nuclease activity is a ther-mostable DNA polymerase having 5′ nuclease activity. Alternatively, the present disclosure may employ a DNA polymerases having 5′ nuclease activity modified to have less polymerase activities.

A suitable DNA polymerase having 5′ nuclease activity in this disclosure is a ther-mostable DNA polymerase obtained from a variety of bacterial species, including Thermus aquaticus (Taq), Thermus thermophilus (Tth), Thermus filiformis, Thermis flavus, Thermococcus literalis, Thermus antranikianii, Thermus caldophilus, Thermus chliarophilus, Thermus flavus, Thermus igniterrae, Thermus lacteus, Thermus oshimai, Thermus ruber, Thermus rubens, Thermus scotoductus, Thermus silvanus, Thermus species Z05, Thermus species sps 17, Thermus thermophilus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosiphoafricanus, Pyrococcus woesei, Pyrococcus horikoshii, Pyrococcus abyssi, Pyrodictium occultum, Aquifex pyrophilus and Aquifex aeolieus. Particularly, the thermostable DNA polymerase is Taq polymerase.

In another embodiment, an enzyme having 5′ nuclease activity and a template-dependent polymerase can be used instead of the DNA polymerase having 5′ nuclease activity. For example, FEN (flap endonuclease) may be used as an enzyme having 5′ nuclease activity.

The FEN is a 5′ flap-specific nuclease.

The FEN suitable in the present disclosure comprises FEN obtained from a variety of bacterial species, including Sulfolobus solfataricus, Pyrobaculum aerophilum, Thermococcus litoralis, Archaeaglobus benefices, Archaeaglobus profundus, Acidianus brierlyi, Acidianus ambivalens, Desulfurococcus amylolyticus, Desulfurococcus mobilis, Pyrodictium brockii, Thermococcus gorgonarius, Thermococcus zilligii, Methanopyrus kandleri, Methanococcus igneus, Pyrococcus horikoshii, Aeropyrum pernix, and Archaeaglobus veneficus.

The template-dependent nucleic acid polymerase may include any nucleic acid polymerases, for example, Klenow fragment of E. coli DNA polymerase I, a thermostable DNA polymerase and bacteriophage T7 DNA polymerase. Particularly, the polymerase is a thermostable DNA polymerase which may be obtained from a variety of bacterial species, including Thermus aquaticus (Taq), Thermus thermophilus (Tth), Thermus filiformis, Thermis flavus, Thermococcus literalis, Thermus antranikianii, Thermus caldophilus, Thermus chliarophilus, Thermus flavus, Thermus igniterrae, Thermus lacteus, Thermus oshimai, Thermus ruber, Thermus rubens, Thermus scotoductus, Thermus silvanus, Thermus species Z05, Thermus species sps 17, Thermus thermophilus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosipho africanus, Pyrococcus furiosus (Pfu), Pyrococcus woesei, Pyrococcus horikoshii, Pyrococcus abyssi, Pyrodictium occultum, Aquifex pyrophilus and Aquifex aeolieus. Most particularly, the template-dependent nucleic acid polymerase is Taq polymerase.

In one embodiment, the conditions for cleavage of the PTO comprise extension reaction of the primer.

In one embodiment, a template-dependent polymerase is used for extension of the primer, and the template-dependent polymerase is identical to the enzyme having 5′ nuclease activity. Alternatively, a template-dependent polymerase is used for extension of the primer, and the template-dependent polymerase is different from the enzyme having the 5′ nuclease activity.

Step (c): Hybridization of Fragment with CTO

The fragment released from the PTO is hybridized with a Capturing and Templating Oligonucleotide (CTO).

The CTO comprises in a 3′ to 5′ direction: (i) a capturing portion comprising a hybridizing nucleotide sequence with the 5′-tagging portion of the PTO, and (ii) a templating portion comprising a non-hybridizing nucleotide sequence with the 5′-tagging portion and the 3′-targeting portion of the PTO.

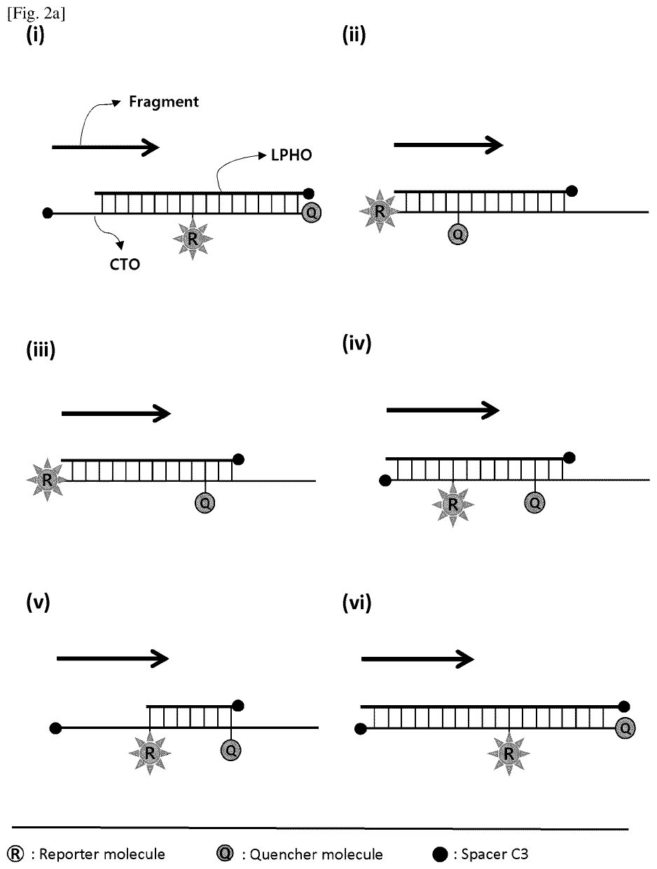

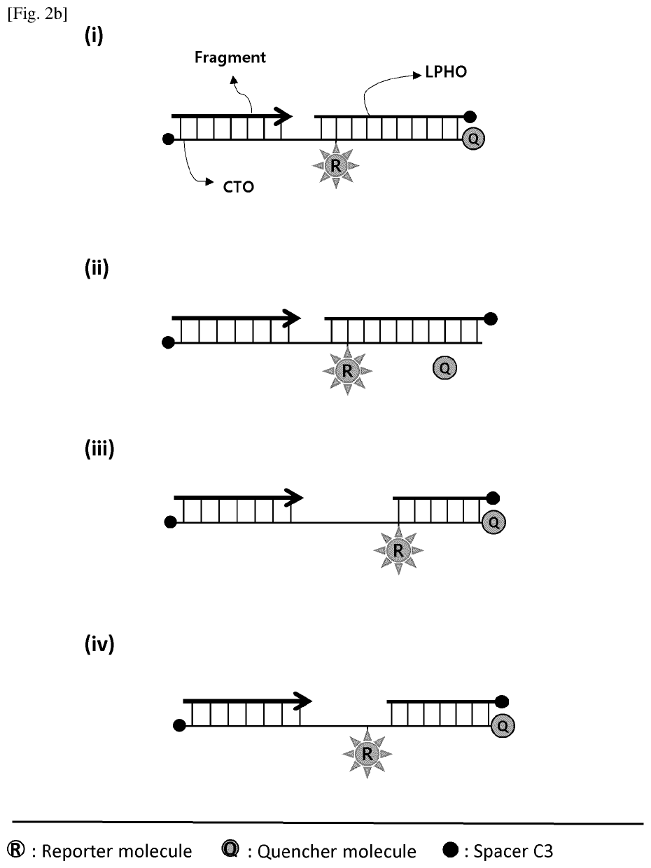

In particular, the CTO used in the present disclosure has a reporter molecule and a quencher molecule defining a labeled portion (see FIG. 2A and FIG. 2B). In other words, the labeled portion may be defined by positions at which the reporter molecule and the quencher molecule are linked.

As used herein, the term “labeled portion” refers to a nucleotide sequence comprising a nucleotide to which the reporter molecule is linked, a nucleotide to which the quencher molecule is linked, and intervening nucleotides. For example, where the reporter molecule and the quencher molecule are linked to the 3rd nucleotide and the 15^{th} nucleotide from the 5′-end of the CTO, respectively, the labeled portion may be a total of 13 nucleotides, including from the 3rd nucleotide to which the reporter molecule is linked to the 15th nucleotide to which the quencher molecule is linked.

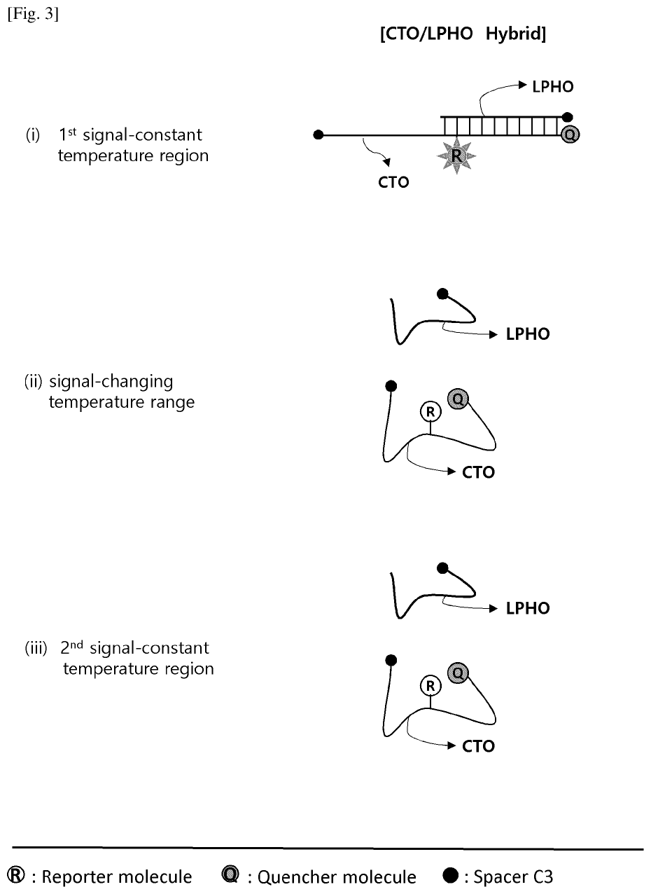

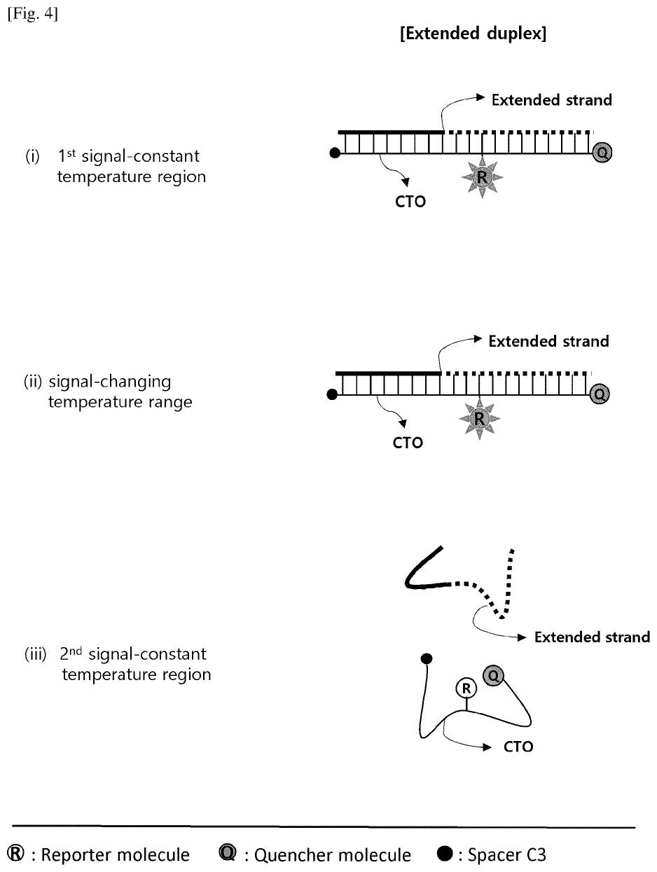

In one embodiment, the reporter molecule and the quencher molecule are located at positions such that (i) the reporter molecule and the quencher molecule are in close proximity to each other, prior to hybridization of the CTO with another oligonucleotide (e.g., LPHO or extended strand), thereby causing the quencher molecule to quench a signal from the reporter molecule (see FIG. 3 (ii) to (iii) and FIG. 4 (iii)), and (ii) the reporter molecule and the quencher molecule are separated from each other, upon hy-bridization of the CTO with the another oligonucleotide, thereby causing the quencher molecule to unquench a signal from the reporter molecule (see FIG. 3 (i) and FIG. 4 (i) to (ii)).

In one embodiment, one of the reporter molecule and the quencher molecule on the CTO is located at its 5′-end or at 1-5 nucleotides apart from its 5′-end and the other is located to quench or unquench the signal from the reporter molecule depending on conformation of the CTO.

In one embodiment, one of the reporter molecule and the quencher molecule on the CTO is located at its 3′-end or at 1-5 nucleotides apart from its 3′-end and the other is located to quench or unquench the signal from the reporter molecule depending on conformation of the CTO.

In one embodiment, the reporter molecule and the quencher molecule are positioned at no more than 80 nucleotides, no more than 60 nucleotides, no more than 30 nu-cleotides, or no more than 25 nucleotides apart from each other. In one embodiment, the reporter molecule and the quencher molecule are separated by at least 4 nu-cleotides, at least 6 nucleotides, at least 10 nucleotides, or at least 15 nucleotides.

In certain embodiments, the reporter molecule and the quencher molecule are separated by 10-25 nucleotides, 10-20 nucleotides, or 10-15 nucleotides.

The positions of the reporter molecule and the quencher molecule should be de-termined, in consideration of a nucleotide sequence of LPHO described later.

The expression herein “the reporter molecule and the quencher molecule are in close proximity to each other” means that (i) an oligonucleotide having both a reporter molecule and a quencher molecule forms a specific conformational structure, for example, a random coil or hairpin structure, so that the reporter molecule and the quencher molecule are three-dimensionally adjacent to each other or (ii) an oligonu-cleotide having the reporter molecule and an oligonucleotide having the quencher molecule form a double strand so that the reporter molecule and the quencher molecule are in close proximity to each other.

In one embodiment, the CTO forms a random coil or hairpin structure, prior to hy-bridization of the CTO with another oligonucleotide, thereby allowing the quencher molecule to intramolecularly quench a signal from the reporter molecule.

The expression herein “the reporter molecule and the quencher molecule are separated from each other” means that (i) an oligonucleotide having both a reporter molecule and a quencher molecule forms a double strand with another oligonucleotide to undergo its conformational change such as disruption of a hairpin structure, so that the reporter molecule and the quencher molecule are separated or (ii) an oligonu-cleotide having the reporter molecule and an oligonucleotide having the quencher molecule in a double stranded state dissociate from each other, so that the reporter molecule and the quencher molecule are separated.

In one embodiment, the CTO forms a double strand upon hybridization of the CTO with another oligonucleotide, thereby allowing the quencher molecule to unquench a signal from the reporter molecule.

In one embodiment, (i) both the reporter molecule and the quencher molecule may be linked to the capturing portion of the CTO, (ii) both the reporter molecule and the quencher molecule may be linked to the templating portion of the CTO, or (iii) one of the reporter molecule and the quencher molecule may be linked to the capturing portion of the CTO and the other may be linked to the templating portion of the CTO.

The reporter molecule and quencher molecule used in this disclosure are interactive labels.

As a representative of the interactive label system, the FRET (fluorescence resonance energy transfer) label system includes a fluorescent reporter molecule (donor molecule) and a quencher molecule (acceptor molecule). In FRET, the energy donor is fluorescent, but the energy acceptor may be fluorescent or non-fluorescent. In another form of interactive label systems, the energy donor is non-fluorescent, e.g., a chromophore, and the energy acceptor is fluorescent. In yet another form of interactive label systems, the energy donor is luminescent, e.g., bioluminescent, chemilu-minescent, electrochemiluminescent, and the acceptor is fluorescent. Donor molecules and acceptor molecules may be described in this disclosure as reporter molecules and quencher molecules, respectively. The interactive label system includes a label pair based on “contact-mediated quenching” (Salvatore et al., Nucleic Acids Research, 2002 (30) no. 21 e122 and Johansson et al., J. AM. CHEM. SOC 2002 (124) pp 6950-6956). The interactive label system includes any label systems that induce signal changes through interactions between at least two molecules (e.g., dyes).

The reporter molecule and the quencher molecule useful in the present invention may include any molecules known in the art. Examples of such molecules include, but not limited to, Cy2™(506), YO-PRO™-1 (509), YOYO™-1 (509), Calcein (517), FITC (518), FluorX™ (519), Alexa™ (520), Rhodamine 110 (520), Oregon Green™ 500 (522), Oregon Green™ 488 (524), RiboGreen™ (525), Rhodamine Green™ (527), Rhodamine 123 (529), Magnesium Green™ (531), Calcium Green™ (533), TO-PRO™-1 (533), TOTO1 (533), JOE (548), BODIPY530/550 (550), Dil (565), BODIPY TMR (568), BODIPY558/568 (568), BODIPY564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAMRA (582), Rhodamine Red™ (590), Cy3.5™ (596), ROX (608), Calcium Crimson™ (615), Alexa™ 594 (615), Texas Red (615), Nile Red (628), YO-PRO™-3 (631), YOYO™-3 (631), Phycocyanin (642), C-Phycocyanin (648), TO-PRO™-3 (660), TOTO3 (660), DiD DilC (5) (665), Cy5™ (670), Thiadicar-bocyanine (671), Cy5.5 (694), HEX (556), TET (536), Biosearch Blue (447), CAL Fluor Gold 540 (544), CAL Fluor Orange 560 (559), CAL Fluor Red 590 (591), CAL Fluor Red 610 (610), CAL Fluor Red 635 (637), FAM (520), Fluorescein (520), Flu-orescein-C3 (520), Pulsar 650 (566), Quasar 570 (667), Quasar 670 (705), and Quasar 705 (610). The numeral in parenthesis is a maximum emission wavelength in nanometer. Preferably, the reporter molecule and the quencher molecule include JOE, FAM, TAMRA, ROX and fluorescein-based label.

Suitable pairs of reporter-quencher are disclosed in a variety of publications as follows: Pesce et al., editors, Fluorescence Spectroscopy (Marcel Dekker, New York, 1971); White et al., Fluorescence Analysis: A Practical Approach (Marcel Dekker, New York, 1970); Berlman, Handbook of Fluorescence Spectra of Aromatic Molecules, 2nd Edition (Academic Press, New York, 1971); Griffiths, Color AND Constitution of Organic Molecules (Academic Press, New York, 1976); Bishop, editor, Indicators (Pergamon Press, Oxford, 1972); Haugland, Handbook of Fluorescent Probes and Research Chemicals (Molecular Probes, Eugene, 1992); Pringsheim, Fluo-rescence and Phosphorescence (Interscience Publishers, New York, 1949); Haugland, R. P., Handbook of Fluorescent Probes and Research Chemicals, 6th Edition (Molecular Probes, Eugene, Oreg., 1996); U.S. Pat. Nos. 3,996,345 and 4,351,760.

A non-fluorescent black quencher molecule capable of quenching a fluorescence of a wide range of wavelengths or a specific wavelength may be used in the present disclosure. Examples of those are BHQ and DABCYL.

In the FRET label adopted to the present disclosure, the reporter encompasses a donor of FRET and the quencher encompasses the other partner (acceptor) of FRET. For example, a fluorescein dye is used as the reporter and a rhodamine dye as the quencher.

The CTO serves as a template for extension of the fragment released from the PTO. The fragment as a primer is hybridized with the CTO and extended to generate an extended duplex.

The templating portion of the CTO may comprise any sequence so long as it is non-complementary to the 5′-tagging portion and the 3′-targeting portion of the PTO. Furthermore, the templating portion of the CTO may comprise any sequence so long as it can serve as a template for extension of the fragment released from the PTO.

As described above, when the fragment comprising the 5′-tagging portion of the PTO is released, it is preferred that the capturing portion of the CTO is designed to comprise a hybridizing nucleotide sequence with the 5′-tagging portion. When the fragment comprising the 5′-tagging portion and a 5′-end part of the 3′-targeting portion of the PTO is released, it is preferred that the capturing portion of the CTO is designed to comprise a hybridizing nucleotide sequence with the 5′-tagging portion and the 5′-end part of the 3′-targeting portion. When the PTO fragment comprising a part of the 5′-tagging portion of the PTO is released, it is preferred that the capturing portion of the CTO is designed to comprise a hybridizing nucleotide sequence with the part of the 5′-tagging portion.

Moreover, it is possible to design the capturing portion of the CTO with anticipating cleavage sites of the PTO. For example, where the capturing portion of the CTO is designed to comprise a hybridizing nucleotide sequence to the 5′-tagging portion, either the fragment comprising a part of the 5′-tagging portion or the fragment comprising the 5′-tagging portion can be hybridized with the capturing portion of the CTO and then extended.

In one embodiment, the nucleotide sequence of the 5′-end part of the capturing portion of the CTO hybridized with the cleaved 5′-end part of the 3′-targeting portion may be selected depending on anticipated cleavage sites on the 3′-targeting portion of the PTO. The nucleotide sequence of the 5′-end part of the capturing portion of the CTO hybridized with the cleaved 5′-end part of the 3′-targeting portion is 1-10 nu-cleotides, 1-5 nucleotides, or 1-3 nucleotides in length.

The term used “capturing portion comprising a nucleotide sequence complementary to the 5′-tagging portion or a part of the 5′-tagging portion” is described herein to encompass various designs and compositions of the capturing portion of the CTO as discussed above.

In one embodiment, the CTO may be designed to have a hairpin structure or no hairpin structure.

The length of the CTO may vary widely. For example, the CTO is 5-1000 nu-cleotides, 5-500 nucleotides, 5-300 nucleotides, 5-100 nucleotides, 5-80 nucleotides, 5-60 nucleotides, 5-40 nucleotides, 7-1000 nucleotides, 7-500 nucleotides, 7-300 nu-cleotides, 7-100 nucleotides, 7-80 nucleotides, 7-60 nucleotides, 7-40 nucleotides, 15-1000 nucleotides, 15-500 nucleotides, 15-300 nucleotides, 15-100 nucleotides, 15-80 nucleotides, 15-60 nucleotides, 15-40 nucleotides, 20-1000 nucleotides, 20-500 nucleotides, 20-300 nucleotides, 20-100 nucleotides, 20-80 nucleotides, 20-60 nu-cleotides, 20-40 nucleotides, 30-1000 nucleotides, 30-500 nucleotides, 30-300 nu-cleotides, 30-100 nucleotides, 30-80 nucleotides, 30-60 nucleotides, or 30-40 nu-cleotides in length.

The capturing portion of the CTO may have any length so long as it is specifically hybridized with the fragment. For example, the capturing portion of the CTO is 5-100 nucleotides, 5-60 nucleotides, 5-40 nucleotides, 5-30 nucleotides, 5-20 nucleotides, 10-100 nucleotides, 10-60 nucleotides, 10-40 nucleotides, 10-30 nucleotides, 10-20 nu-cleotides, 15-100 nucleotides, 15-60 nucleotides, 15-40 nucleotides, 15-30 nucleotides, or 15-20 nucleotides in length.

The templating portion of the CTO may have any length so long as it can act as a template in extension of the fragment. For example, the templating portion of the CTO is 1-900 nucleotides, 1-400 nucleotides, 1-300 nucleotides, 1-100 nucleotides, 1-80 nu-cleotides, 1-60 nucleotides, 1-40 nucleotides, 1-20 nucleotides, 2-900 nucleotides, 2-400 nucleotides, 2-300 nucleotides, 2-100 nucleotides, 2-80 nucleotides, 2-60 nu-cleotides, 2-40 nucleotides, 2-20 nucleotides, 5-900 nucleotides, 5-400 nucleotides, 5-300 nucleotides, 5-100 nucleotides, 5-80 nucleotides, 5-60 nucleotides, 5-40 nu-cleotides, 5-30 nucleotides, 10-900 nucleotides, 10-400 nucleotides, 10-300 nu-cleotides, 15-900 nucleotides, 15-100 nucleotides, 15-80 nucleotides, 15-60 nu-cleotides, 15-40 nucleotides, or 15-20 nucleotides in length.

In one embodiment, the 3′-end of the CTO may have a 3′-OH terminal. Alternatively, the 3′-end of the CTO is blocked to prohibit its extension. The blocking of the CTO can be described in detail with referring to descriptions of the PTO's blocking described above.

The fragment is hybridized with the CTO, providing a form suitable in extension of the fragment. Although an uncleaved PTO is also hybridized with the capturing portion of the CTO through its 5′-tagging portion, its 3′-targeting portion is not hybridized with the CTO, which prohibits generation of an extended duplex.

The hybridization in the step (c) can be described in detail with referring to de-scriptions in the step (a).

### Step (d): Extension of Fragment and Generation of Extended Duplex

The extension reaction is carried out using the resultant of the step (c) and the DNA polymerase having 5′ nuclease activity in the presence of a labeled portion hybridizing oligonucleotide (LPHO). The fragment hybridized with the capturing portion of the CTO is extended to generate an extended strand comprising an extended sequence complementary to the templating portion of the CTO. This results in an extended duplex between the extended strand and the CTO. In contrast, uncleaved PTO hybridized with the capturing portion of the CTO is not extended such that no extended duplex is generated.

In the step (d), the fragment hybridized with the capturing portion of the CTO is extended along the templating portion of the CTO as a template by a DNA polymerase having 5′ nuclease activity.

The terms “extended sequence”, “extended strand” and “extended duplex” as used in connection with the extension reaction of the fragment in step (d) have the following meanings:
As used herein, the term “extended sequence” refers to a sequence which is newly generated by extension from the fragment in the step (d). In other words, the extended sequence refers to a portion of the extended strand as will be described below, excluding the fragment.

As used herein, the term “extended strand” refers to a sequence encompassing the fragment and the extended sequence. In other words, the extended strand refers to a portion of the extended duplex as will be described below, excluding the CTO.

As used herein, the term “extended duplex” refers to a hybrid or duplex (through complementarity) between the extended strand and the CTO. In other words, the extended duplex means a duplex between the extended strand, composed of the fragment and the extended sequence, and the CTO.

In one embodiment, Tm of the extended duplex is adjustable by (i) a sequence and/or length of the fragment, (ii) a sequence and/or length of the CTO or (iii) the sequence and/or length of the fragment and the sequence and/or length of the CTO.

The term used herein “Tm” refers to a melting temperature at which half a population of double stranded nucleic acid molecules are dissociated into single-stranded molecules. The Tm value is determined by length and G/C content of nucleotides hybridized. The Tm value may be calculated by conventional methods such as Wallace rule (R. B. Wallace, et al., Nucleic Acids Research, 6:3543-3547 (1979)) and nearest-neighbor method (SantaLucia J. Jr., et al., Biochemistry, 35:3555-3562 (1996); Sugimoto N., et al., Nucleic Acids Res., 24:4501-4505 (1996)).

In certain embodiments, the Tm value refers to actual Tm values under reaction conditions.

The step (d) is carried out in the presence of Labeled Portion Hybridizing Oligonu-cleotide (LPHO).

The term “Labeled Portion Hybridization Oligonucleotide (LPHO)” as used herein refers to an oligonucleotide which comprises a hybridizing nucleotide sequence with a labeled portion and which provides signals of different intensities depending on whether it is hybridized with the labeled portion. For example, when the LPHO is hybridized with a labeled portion of the CTO, the reporter molecule and the quencher molecule on the CTO are separated, thereby causing the quencher molecule to unquench a signal from the reporter molecule, whereas when the LPHO is not hybridized with the labeled portion of CTO and the CTO is not hybridized with any oligonucleotide, the reporter molecule and the quencher molecule on the CTO are in close proximity to each other, thereby causing the quencher molecule to quench a signal from the reporter molecule.

In one embodiment, the LPHO may be hybridized with a complete or a partial sequence of the labeled portion of the CTO, as long as the LPHO provides signals of different intensities, depending on whether the LPHO is hybridized with the labeled portion of CTO.

In one embodiment, the LPHO is hybridized with a complete or a partial sequence of the labeled portion of the CTO, and the reporter molecule and the quencher molecule on the CTO are separated, thereby causing the quencher molecule to unquench a signal from the reporter molecule.

The LPHO comprises a hybridizing nucleotide sequence with the labeled portion of the CTO. For example, where both the reporter molecule and the quencher molecule are linked to the capturing portion (or templating portion) of the CTO, the LPHO should be designed to comprise a nucleotide sequence that hybridizes with the capturing portion (or templating portion) of the CTO to which the reporter molecule and the quencher molecule are linked. In this case, it is to be understood by one skilled in the art that the LPHO may have additional nucleotide sequences in addition to the hybridizing nucleotide sequence with the labeled portion of the CTO described above.

In certain embodiments, when the reporter molecule is linked to the 12th nucleotide from the 5′-end of the CTO and the quencher molecule is linked to the 25th nucleotide from the 5′-end of the CTO, the LPHO may comprise a nucleotide sequence com-plementary to a nucleotide sequence from the 12th nucleotide to the 25th nucleotide from the 5′-end on the CTO defined as the labeled portion.

The length of the LPHO may vary widely. For example, the LPHO is 5-100 nu-cleotides, 5-80 nucleotides, 5-60 nucleotides, 5-40 nucleotides, 5-20 nucleotides, 5-10 nucleotides, 7-100 nucleotides, 7-80 nucleotides, 7-60 nucleotides, 7-40 nucleotides, 7-20 nucleotides, 7-10 nucleotides, 10-100 nucleotides, 10-80 nucleotides, 10-60 nu-cleotides, 10-40 nucleotides, 10-30 nucleotides, 10-20 nucleotides, 10-15 nucleotides, 15-100 nucleotides, 15-80 nucleotides, 15-60 nucleotides, 15-40 nucleotides, 15-30 nu-cleotides, 15-20 nucleotides, 20-100 nucleotides, 20-80 nucleotides, 20-60 nucleotides, 20-40 nucleotides, or 20-30 nucleotides in length.

One of the features of the present disclosure is that when the target nucleic acid is present in the sample, the extended duplex is generated between the extended strand and the CTO, thereby preventing the formation of the CTO/LPHO hybrid between the labeled portion of the CTO and the LPHO; when the target nucleic acid is not present in the sample, the extended strand is not generated (i.e., the extended duplex is not generated), and instead the CTO/LPHO hybrid is formed.

In one embodiment, when the target nucleic acid is present in the sample, the fragment hybridized with the capturing portion of the CTO is extended to generate the extended strand comprising the extended sequence complementary to the templating portion of the CTO, thereby generating the extended duplex between the extended strand and the CTO.

In one embodiment, when the target nucleic acid is present in the sample, the extended duplex may be generated by (i) extending the fragment hybridized to the capturing portion of the CTO prior to the hybridization of the labeled portion of the CTO and the the LPHO, (ii) extending the fragment hybridized to the capturing portion of the CTO upon the hybridization between the labeled portion of the CTO and the LPHO, thereby cleaving the LPHO, or (iii) both (i) and (ii).

The LPHO may be hybridized with the CTO prior to the extension of the fragment and involved in the extension reaction. In one embodiment, when the LPHO is hybridized with the CTO prior to the extension of the fragment, the extension of the fragment cleaves or displaces the LPHO from the CTO. For example, as the fragment is extended, the LPHO hybridized with the CTO may be released (displaced) from CTO by strand displacement or may be cleaved.

In one embodiment, the cleavage and/or strand displacement of the LPHO by the extension of the fragment is dependent on types of enzymes (e.g., DNA polymerase) or reaction conditions.

In one embodiment, the generation of the extended duplex prevents the formation of the CTO/LPHO hybrid, via preference for hybridization between the extended strand and the CTO, i.e., formation of the extended duplex over hybridization between the labeled portion of the CTO and the LPHO, i.e., formation of the CTO/LPHO hybrid. In other words, the CTO is hybridized with the extended strand to generate the extended duplex, which results in consumption of the CTO and low possibility of hybridization between the CTO and the LPHO.

In one embodiment, the generation of extended duplex prevents the formation of the CTO/LPHO hybrid, via cleavage of the LPHO during the extension of step (d). In other words, the LPHO is cleaved and removed, which results in low possibility of hy-bridization between the CTO and the LPHO.

In one embodiment, the extended duplex has a melting temperature (Tm) that is different from a Tm of the CTO/LPHO hybrid.

In particular, the preference for the formation of the extended duplex over the formation of the CTO/LPHO hybrid can be achieved through differences in the stability of the extended duplex and the CTO/LPHO hybrid, such as differences in Tm values.

In the present disclosure, the extended duplex may be more stable than the CTO/LPHO hybrid. For example, the Tm of the extended duplex is higher than the Tm of the CTO/LPHO hybrid. Particularly, the Tm of the extended duplex is at least 2° C., 3° C., 4° C., 5° C., 7° C., 10° C., 15° C., or 20° C. higher than the Tm of the CTO/LPHO hybrid.

In one embodiment, the 3′-end of the LPHO is blocked to prohibit its extension. The blocking of the LPHO can be described in detail with referring to descriptions of the PTO's blocking described above.

The LPHO to be hybridized with the labeled potion of the CTO may be located relative to the PTO fragment to be hybridized with the capturing portion of the CTO in any of two fashions: (i) the LPHO completely or partially overlaps with the PTO fragment; and (ii) the LPHO does not overlap with the PTO fragment.

The above aspects (i) and (ii) will be described with reference to FIGS. 2A and 2B.

(i) where the LPHO to be Hybridized with the Labeled Portion of the CTO Completely or Partially Overlaps with the PTO Fragment to be Hybridized with the Capturing Portion of the CTO

In this case, the LPHO comprises a nucleotide sequence which competes with the fragment for hybridization with the CTO. For example, the LPHO that comprises a nucleotide sequence hybridizable with all or a part of the capturing portion of the CTO may compete with the fragment for hybridization with the CTO.

The expression herein “the LPHO comprises a nucleotide sequence which competes with the fragment for hybridization with the CTO” means that the LPHO comprises a nucleotide sequence hybridizable with the same portion to which the fragment is hybridized. The same portion is used to encompass a portion that is partially or completely identical to the portion to which the fragment is hybridized. In other words, the LPHO may comprise a nucleotide sequence that completely or partially overlaps with the 5′-tagging portion of PTO (see FIG. 2A).

The term “a nucleotide sequence hybridizable with the capturing portion of the CTO” as used herein in conjunction with a sequence of the LPHO refers to a portion of the LPHO to form a double strand by hybridization with the capturing portion of the CTO. The nucleotide sequence of the LPHO hybridizable with the capturing portion of the CTO may be complete sequence or a partial sequence of the LPHO. The nucleotide sequence hybridizable with the capturing portion of the CTO corresponds to complete or a partial sequence (e.g., 10%, 30%, 40%, 50%, 60%, 70%, 80%, 90%, or 95%) of the LPHO.

In one embodiment, where the LPHO comprises a nucleotide sequence which competes with the fragment for hybridization with the CTO, the LPHO is not cleaved or displaced by the fragment or its extension product during the extension reaction.

In one embodiment, where the LPHO comprises a nucleotide sequence which competes with the fragment for hybridization with the CTO, the LPHO is less com-petitive than the fragment (specifically, the extended strand of the fragment) and more competitive than the 5′-tagging portion of the uncleaved PTO in terms of hybridization with the CTO.

In certain embodiments, the step (d) is performed under conditions that are more favorable to hybridization between the fragment and the CTO than hybridization between the LPHO and the CTO. Such favorable conditions may be accomplished by various methods. For example, the 3′-end of the LPHO may be blocked for the favorable conditions. The LPHO with blocked 3′-end is hybridized with the CTO but is not extended, which increases probability of dissociation from the CTO due to com-petition with the fragment. The fragment hybridized with the CTO is extended to generate the extended strand, which may be more stably maintained. Therefore, the extended duplex is much more prevalent than the CTO/LPHO hybrid in the step (d). Consequently, the number (or amount) of the CTO/LPHO hybrid is relatively decreased due to the extended duplex in the presence of the target nucleic acid compared with in the absence of the target nucleic acid.

Where the target nucleic acid is absent, the cleavage of the PTO does not occur and the PTO exists as uncleaved PTO. Where both the uncleaved PTO and the LPHO exist, the 5′-tagging portion of the uncleaved PTO competes with the LPHO for hy-bridization with the CTO, because they have an overlapping sequence with each other. Where the target nucleic acid is absent, the LPHO should be more favorable for hy-bridization with the CTO than the 5′-tagging portion of the uncleaved PTO, because the underlying principle of the present disclosure requires the hybridization of the LPHO with the CTO. For example, where the Tm value of the fragment is higher than that of the LPHO, the fragment is more favorable for hybridization with the CTO than the LPHO.

In one embodiment, the LPHO may comprise a hybridizing sequence with the complete sequence of the CTO (see FIG. 2A, (vi)). In other words, the LPHO may be of the same length as the extended strand. In this case, the LPHO may be designed to have non-natural bases or to have some mismatches to the CTO, so that hybridization of the CTO with the extended strand is more favorable than that with the LPHO.

The LPHO should be suitably designed, considering factors or issues described above. In one embodiment, the difference between the Tm values of the CTO/LPHO hybrid and the CTO/fragment hybrid is within ±40° C., ±30° C., ±20° C., ±15° C., ±10° C., ±5° C., ±3° C., or ±1° C.

In one embodiment, the difference between the Tm values of the CTO/LPHO hybrid and the CTO/5′-tagging portion of the uncleaved PTO hybrid is within ±40° C., ±30° C., ±20° C., ±15° C., ±10° C., ±5° C., ±3° C., or ±1° C.

In one embodiment, where the LPHO competes with the uncleaved PTO for hy-bridization with the CTO, the Tm value of the CTO/LPHO hybrid may be higher (e.g., at least 2° C., 4° C., 6° C., 8° C., 10° C., 15° C. or 20° C. higher) than that of the CTO/uncleaved PTO hybrid.

The Tm value of the CTO/uncleaved PTO hybrid is determined by a portion of the PTO to be hybridized with CTO. For example, where the 5′-tagging portion of the uncleaved PTO is to be hybridized with the CTO, the Tm value of the 5′-tagging portion is a determinative factor for the Tm value of the CTO/uncleaved PTO hybrid.

The term “Tm value of the uncleaved PTO” as used herein means a Tm value de-termined by a portion of the uncleaved PTO sequence to be hybridized with the CTO, unless otherwise indicated.

In one embodiment, the extended strand has a Tm value higher than the LPHO, which has a Tm value higher than the 5′-tagging portion of the PTO.

In one embodiment, given hybridization with CTO, the Tm value of the extended strand is higher than that of the LPHO, which is higher than that of the uncleaved PTO.

(ii) where the LPHO to be Hybridized with the Labeled Portion of the CTO does not Overlap with the PTO Fragment to be Hybridized with the Capturing Portion of the CTQ

In one embodiment, the LPHO may be designed to comprise a hybridizing nucleotide sequence with a different portion to which the fragment is hybridized. For example, the LPHO comprises a hybridizing nucleotide sequence with the templating portion of the CTO, and the fragment and the LPHO are hybridized with different portions on the CTO as shown in FIG. 2B, (i) to (iii). In this case, the LPHO may not compete with the fragment (or uncleaved PTO) in terms of hybridization with CTO.

In one embodiment, the hybridization between the LPHO and the CTO may or may not be favorable than non-hybridization, depending on conditions for extension of the fragment hybridized with the CTO.

In one embodiment, the fragment hybridized with the CTO may be extended prior to the hybridization of the labeled portion of the CTO and the LPHO.

In one embodiment, the fragment hybridized with the CTO may be extended upon the hybridization between the labeled portion of the CTO and the LPHO. In this case, the LPHO hybridized with the labeled portion of the CTO is released (separated) or cleaved from the CTO by the fragment or its extension product.

### Step (e): Detection of Presence of Extended Duplex

Finally, the presence of the extended duplex is detected. The presence of the extended duplex indicates the presence of the target nucleic acid.

The step (e) can be accomplished by detecting a signal indicative of the presence of the extended duplex.

The term “signal” as used herein means any signal capable of indicating the presence of the extended duplex. For example, the signal includes a signal change from labels (signal generation or extinguishment, or signal increase or decrease), a melting curve, a melting pattern, and a melting temperature (or Tm value).

In one embodiment, a signal is provided from the extended duplex and the extended duplex is detected by measuring the signal provided from the extended duplex. The measurement is performed at a temperature at which the signal intensity provided from the extended duplex is different from a signal intensity provided from the CTO/LPHO hybrid. For example, at the temperature for measurement, the signal from the reporter molecule is unquenched or quenched, depending on whether it is the extended duplex or the CTO/LPHO hybrid, providing signals of different intensities.

In one embodiment, the signal provided from the extended duplex is a signal upon association of the CTO and the extended strand into the extended duplex or dis-sociation of the extended duplex into the CTO and the extended strand. Particularly, the signal provided from the extended duplex is a signal upon association of the CTO and the extended strand into the extended duplex.

The term “association” or “dissociation” has the same meaning as the term “hy-bridization” or “denaturation”, respectively.

In another embodiment, a signal is provided from the CTO/LPHO hybrid and the extended duplex is detected by measuring the signal provided from the CTO/LPHO hybrid. As described above, the generation of the extended duplex prevents the formation of the CTO/LPHO hybrid, which changes the signal provided from the CTO/LPHO hybrid. Therefore, the presence of the extended duplex can be detected by measuring this signal change provided from the CTO/LPHO hybrid.

In one embodiment, the Tm of the extended duplex is adjustable by (i) a sequence and/or length of the fragment, (ii) a sequence and/or length of the CTO, or (iii) the sequence and/or length of the fragment and the sequence and/or length of the CTO, and the Tm of the CTO/LPHO hybrid is adjustable by a sequence and/or length of the LPHO.

In one embodiment, the temperature for measurement depends on the Tm of the extended duplex and the Tm of the CTO/LPHO hybrid.

In one embodiment, the method is performed in the presence of a plurality of the PTOs, a plurality of the CTOs, and a plurality of the LPHOs, and the steps (a)-(e) are repeated with denaturation between repeating cycles.

In the method according to the present disclosure, the extended duplex is generated depending on the presence of a target nucleic acid, and the amount of the extended duplex increases as the reaction progresses. On the other hand, the amount of the CTO/LPHO hybrid decreases as the extended duplex is generated depending on the presence of target nucleic acid. This change in the amount of the extended duplex or CTO/LPHO hybrid provides a change in signal indicating the presence of the target nucleic acid. In other words, the amount ratios of the extended duplex and CTO/LPHO hybrid changes depending on the presence of the target nucleic acid, thereby changing the signal.

The term “amount” as used herein when referring to the extended duplex or the CTO/LPHO hybrid refers to the amount of the two nucleic acid strands that make up the duplex or hybrid. In one embodiment, the two nucleic acid strands constituting the duplex may be dissociated or associated depending on the temperature. In this case, the amount of the duplex refers to the sum of the amount of duplexes in dissociated form and the amount of duplexes in associated form.

In one embodiment, when a target nucleic acid is present, the CTO may be hybridized with the extended strand to form the extended duplex or hybridized with the LPHO to form the CTO/LPHO hybrid. In this case, the amount of the extended duplex is calculated based on the amount of the extended strand, and then the amount of the CTO/LPHO hybrid is calculated based on the amount of remaining CTO excluding the CTO hybridized with the extended strand. For example, before reaction with the target nucleic acid, the amount of the CTO/LPHO hybrid may be calculated based on the as-sumption that all CTOs in the composition for detecting the target nucleic acid are involved in forming the CTO/LPHO hybrid. On the other hand, when the extended strand is generated depending on the presence of the target nucleic acid, the amount of extended duplex and the amount of CTO/LPHO hybrid may be calculated based on the assumption that most of the CTOs in the composition for detecting the target nucleic acid are preferentially hybridized with the extended strand to form the extended duplex, and the remaining CTOs are hybridized with the LPHO to form the CTO/LPHO hybrid.

The detection of the presence of the extended duplex in the step (e) may be performed by (i) measuring a signal at a predetermined temperature, or (ii) measuring a signal by melting analysis or a melting followed by hybridization analysis.

### (i) Measuring a Signal at a Predetermined Temperature

The detection in step (e) is carried out by measuring a signal indicative of the presence of the extended duplex at a predetermined temperature. For example, the pre-determined temperature is a temperature at which the signal intensity provided from the extended duplex is different from a signal intensity provided from the CTO/LPHO hybrid.

For example, at a predetermined temperature, the quencher molecule on the extended duplex unquenches the signal from the reporter molecule on the extended duplex, while the quencher molecule on the CTO/LPHO hybrid quenches the signal from the reporter molecule on CTO/LPHO hybrid, or vice versa. That is, the signal is un-quenched or quenched depending on whether the quencher molecule is on the extended duplex or the CTO/LPHO hybrid at a predetermined temperature, thereby providing signals of different intensities.

In one embodiment, the temperature for measurement allows both (i) at least one of the extended duplexes to remain its double-stranded state and (ii) at least one of the CTO/LPHO hybrids to dissociate into a single-stranded state.

In particular, the signal is measured at a temperature within a temperature range at which all or some of the CTO/LPHO hybrids exists in dissociated form and all or some of the extended duplexes exists in associated form. In other words, the temperature at which all of the CTO/LPHO hybrids and the extended duplexes exist in associated form (e.g., a temperature at least 2° C., 3° C., 4° C., 6° C., 8° C., or 10° C. lower than the Tm value of the CTO/LPHO hybrid) or the temperature at which all of the CTO/LPHO hybrids and the extended duplexes exist in dissociated form (e.g., a temperature at least 2° C., 3° C., 4° C., 6° C., 8° C., or 10° C. higher than the Tm value of the extended duplex), no signal indicating the presence of extended duplex is detected.

Regarding the expression “all or some of the duplexes (e.g., the CTO/LPHO hybrids or the extended duplexes) exist in dissociated form (or associated form) within a specified temperature range (or at specified temperatures)”, the term “all” is used to mean all or substantially all duplexes, such as a significant number of duplexes or most duplexes, within a specified temperature range. For example, the expression “all extended duplexes exist in dissociated form at a temperature 4° C. or more higher than the Tm value of the extended duplex” may mean that most extended duplexes are dis-sociated at a temperature 4° C. or more higher than the Tm value of the extended duplex.

Regarding the expression “all or some of the duplexes (e.g., the CTO/LPHO hybrids or the extended duplexes) exist in dissociated form (or associated form) within a specified temperature range (or at specified temperatures)”, the term “some” refers to a portion of the total amount of the duplex, such as at least 5%, at least 10%, at least 20%, at least 30%, at least 40%, at least 50%, at least 60%, at least 70%, or at least 80% of the total amount of the duplex.

In one embodiment, the temperature range in which all or some of the CTO/LPHO hybrids are in dissociated form and all or some of the extended duplexes are in associated form is from 10° C. lower than the Tm value of the CTO/LPHO hybrid to 10° C. higher than the Tm value of the extended duplex.

In one embodiment, the temperature at which all or some of the CTO/LPHO hybrids are in dissociated form is within +2° C., +3° C., +4° C., +6° C., +8° C. or +10° C. relative to the Tm value of the CTO/LPHO hybrid.

In one embodiment, the temperature at which all or some of the extended duplexes are in associated form is within +2° C., +3° C., +4° C., +6° C., +8° C. or +10° C. relative to the Tm value of the extended duplex.

In one embodiment, the signal indicating the presence of the extended duplex is measured at a temperature within a temperature range that is higher than the Tm value of the CTO/LPHO hybrid and lower than the Tm value of the extended duplex.

In certain embodiments, the signal is measured at a temperature within the temperature range at which all of the CTO/LPHO hybrids are in dissociated form and all of the extended duplexes are in associated form. This temperature range is from 4° C. or higher relative to the Tm value of the CTO/LPHO hybrid to 4° C. or lower relative to the Tm value of the extended duplex.

In certain embodiments, the signal is measured at a temperature within a temperature range at which all the CTO/LPHO hybrids are in dissociated form and some of the extended duplexes are in associated form. This temperature range is 4° C. or higher relative to the Tm value of the CTO/LPHO hybrid and is within +4° C. relative to the Tm value of the extended duplex.

In certain embodiments, the signal is measured at a temperature within a temperature range at which some of the CTO/LPHO hybrids are in dissociated form and some of the extended duplexes are in associated form. This temperature range is within +4° C. relative to the Tm value of the CTO/LPHO hybrid and is 4° C. or lower relative to the Tm value of the extended duplex.

In one embodiment, the temperature for signal measurement depends on the Tm of the extended duplex and the Tm of the CTO/LPHO hybrid.

As described above, the Tm of the extended duplex and the Tm of the CTO/LPHO hybrid are different from each other. Particularly, the Tm value of the extended duplex is higher than that of the CTO/LPHO hybrid. The difference in Tm values allows a change in signal to be provided depending on the presence of target nucleic acid only within a specific temperature range. At temperatures lower or higher than the specific temperature range, the signal is constant even if the target nucleic acid is present.

In this regard, more details are found in the second aspect of the present disclosure, “Composition for detecting a target nucleic acid” and the third aspect of the present disclosure, “Method for detecting n target nucleic acids in a sample.”

### (ii) Measuring a Signal by Melting Analysis or a Melting Followed by Hybridization Analysis.

In one embodiment, the detection in the step (e) is carried out by measuring a signal indicative of the presence of the extended duplex by a melting analysis or a melting followed by hybridization analysis.

In one embodiment, the extended duplex and/or the CTO/LPHO hybrid is melted or hybridized within a certain temperature range, and then the presence of the extended duplex in the step (e) is detected by measuring a signal from the extended duplex and/or a signal from the CTO/LPHO hybrid. Particularly, the result of step (d) (e.g., the extended duplex and/or the CTO/LPHO hybrid) is melted to provide a signal or melted followed by hybridization to provide a signal, and then the presence of the extended duplex is detected by measuring the signal.

In one embodiment, the detection of the presence of the extended duplex in the step (e) is performed by melting analysis, in which the extended duplex is melted to give a signal indicative of the presence of the target nucleic acid.

The term “melting analysis” as used herein means a method in which a signal in-dicative of the presence of the extended duplex is obtained by melting the extended duplex, including melting curve analysis, melting pattern analysis, and melting peak analysis. Particularly, the melting analysis is a melting curve analysis.

In one embodiment, the detection of the extended duplex in the step (e) is carried out by a melting followed by hybridization analysis. Particularly, the detection of the extended duplex in the step (e) is carried out by melting the extended duplex and/or the CTO/LPHO hybrid and hybridizing the resultant at a certain temperature to give a signal indicative of the extended duplex.

The term “melting followed by hybridization analysis” as used herein refers to a method of melting the extended duplex and then hybridizing the melted, extended duplex again, to give a signal indicative of the extended duplex. Particularly, the melting followed by hybridization analysis is a melting curve analysis.

The melting curve or hybridization curve may be obtained by conventional tech-nologies, for example, as described in U.S. Pat. Nos. 6,174,670 and 5,789,167, Drobyshev et al, Gene 188: 45(1997); Kochinsky and Mirzabekov Human Mutation 19:343(2002); Livehits et al J. Biomol. Structure Dynam. 11:783(1994); and Howell et al Nature Biotechnology 17:87(1999). For example, a melting curve or hybridization curve may consist of a graphic plot or display of the variation of the output signal with the parameter of hybridization stringency. Output signal may be plotted directly against the hybridization parameter. Typically, a melting curve or hybridization curve will have the output signal, for example fluorescence, which indicates the degree of duplex structure (i.e. the extent of hybridization), on the Y-axis and the hybridization parameter on the X axis.

A plot of the first derivative of fluorescence versus temperature, i.e., a plot of the rate of change in fluorescence vs. temperature (dF/dT vs. T or dF/dT vs. T) provides a melting peak.

In one embodiment, the method of the present disclosure may further comprise repeating all or a portion of steps (a)-(e) with denaturation between repeating cycles. The repetition results in amplification of the target nucleic acid and/or amplification of a signal indicating the presence of the target nucleic acid.

In one embodiment, the repetition step involving denaturation may comprise at least denaturing the extended duplex. Other components (e.g., primers, downstream primers, and enzymes) may be used in sufficient amounts so as not to be limiting factors.

In one embodiment, the composition for detecting a target nucleic acid according to the present disclosure comprises the LPHOs in an amount equal to or greater than the amount of the CTO. This is to ensure that all the CTOs in the composition form the CTO/LPHO hybrids so that no single-stranded CTO exists. Particularly, the amount of the CTO/LPHO hybrid initially included in the L-PTOCE composition decreases as an extended duplex is generated depending on the presence of the target nucleic acid and provides a signal indicating the presence of the extended duplex. In one embodiment, the LPHO is included in the L-PTOCE composition in an amount of 1, 2, 3, 4, 5 or more times that of the CTO.

The denaturation may be carried out by conventional technologies, including, but not limited to, heating, alkali, formamide, urea and glycoxal treatment, enzymatic methods (e.g., helicase action), and binding proteins. For instance, the denaturation can be achieved by heating at a temperature ranging from 80° C. to 105° C. General methods for accomplishing this treatment are provided by Joseph Sambrook, et al., Molecular Cloning, A Laboratory Manual, Cold Spring Harbor Laboratory Press, Cold Spring Harbor, N.Y. (2001).

The detection of the step (e) may be performed in a real-time manner, an end-point manner, or a predetermined time interval manner. Where the present disclosure further comprises repeating the steps (a)-(e), it is preferred that the signal detection is performed for each cycle of the repetition at a predetermined temperature (i.e. real-time manner), at the end of the repetition at a predetermined temperature (i.e. end-point manner), or at each of predetermined time intervals during the repetition at a prede-termined temperature. Preferably, the detection may be performed for each cycle of the repetition in a real-time manner to improve the detection accuracy and quantification.

In the repetition, the method of the present disclosure is performed in the presence of a downstream primer, particularly by a real-time PCR method.

In one embodiment, the steps (a)-(e) are performed in a reaction vessel or some of the steps (a)-(e) are performed in separate reaction vessels.

The present disclosure does not require that a target nucleic acid to be detected and/or amplified have any particular sequence or length, including any DNA (gDNA and cDNA) and RNA molecules.

Where an mRNA is employed as a starting material, a reverse transcription step is necessary prior to performing annealing step, details of which are found in Joseph Sambrook, et al., Molecular Cloning, A Laboratory Manual, Cold Spring Harbor Laboratory Press, Cold Spring Harbor, N.Y. (2001); and Noonan, K. F. et al., Nucleic Adds Res. 16:10366 (1988). For reverse transcription, a random hexamer or an oligonucleotide dT primer hybridizable to mRNA can be used.

In one embodiment, the target nucleic acid used in the present disclosure is a pre-amplified nucleic acid. The utilization of the pre-amplified nucleic acid permits to sig-nificantly increase the sensitivity and specificity of target detection of the present disclosure.

The present disclosure is also useful in detection of a nucleotide variation. Par-ticularly, the target nucleic acid comprises a nucleotide variation. The term “nucleotide variation” as used herein refers to any single or multiple nucleotide substitutions, deletions, or insertions in a DNA sequence at a particular location among contiguous DNA segments that are otherwise similar in sequence. Such contiguous DNA segments include a gene or any other portion of a chromosome. These nucleotide variations may be mutant or polymorphic allele variations. For example, the nucleotide variation detected in the present disclosure includes SNP (single nucleotide polymorphism), mutation, deletion, insertion, substitution, and translocation. Exemplified nucleotide variation includes numerous variations in a human genome (e.g., variations in the MTHFR (methylenetetrahydrofolate reductase) gene), variations involved in drug re-sistance of pathogens and tumorigenesis-causing variations. The term “nucleotide variation” as used herein includes any variation at a particular location in a nucleic acid sequence. In other words, the term “nucleotide variation” includes a wild type and any mutant type at a particular location in a nucleic acid sequence.

### II. Composition for Detecting Target Nucleic Acid

In a second aspect of the present disclosure, there is provided a composition for detecting a target nucleic acid in a sample, comprising:
(a) a primer;
wherein the primer comprises a hybridizing nucleotide sequence with a first region of the target nucleic acid,
(b) a Probing and Tagging Oligonucleotide (PTO);
wherein the PTO comprises in a 5′ to 3′ direction: (i) a 5′-tagging portion, and (ii) a 3′-targeting portion,
wherein the 3′-targeting portion comprises a hybridizing nucleotide sequence with a second region of the target nucleic acid, and the 5′-tagging portion comprises a nucleotide sequence not hybridized with the target nucleic acid when the 3′-targeting portion is hybridized with the second region of the target nucleic acid,
wherein the primer is located upstream of the PTO,
wherein the primer is extended to induce cleavage of the PTO by a DNA polymerase having 5′ nuclease activity such that the cleavage releases a fragment comprising the 5′-tagging-portion of the PTO;
(c) a Capturing and Templating Oligonucleotide (CTO);
wherein the CTO comprises in a 3′ to 5′ direction: (i) a capturing portion comprising a hybridizing nucleotide sequence with the 5′-tagging portion of the PTO, and (ii) a templating portion comprising a non-hybridizing nucleotide sequence with the 5′-tagging portion and the 3′-targeting portion of the PTO,
wherein the CTO has a reporter molecule and a quencher molecule defining a labeled portion,
wherein the fragment is hybridized with the capturing portion of the CTO; and
(d) a Labeled Portion Hybridizing Oligonucleotide (LPHO);
wherein the LPHO comprises a hybridizing nucleotide sequence with the labeled portion of the CTO,
wherein when the target nucleic acid is present in the sample, the fragment hybridized with the capturing portion of the CTO is extended to generate an extended strand complementary to the CTO, thereby generating an extended duplex between the extended strand and the CTO, wherein the generation of the extended duplex prevents the formation of a CTO/LPHO hybrid between the labeled portion of the CTO and the LPHO,
wherein when the target nucleic acid is not present in the sample, the extended strand is not generated, and instead the CTO/LPHO hybrid is formed,
wherein the extended duplex has a melting temperature (Tm) that is different from a Tm of the CTO/LPHO hybrid.

Since the second aspect of the present disclosure follows the principles of the first aspect of the present disclosure described above, the common descriptions between them are omitted in order to avoid undue redundancy leading to the complexity of this specification.

The composition according to the present disclosure is constructed to perform the method for detecting a target nucleic acid by L-PTOCE assay described above, which is termed “LPHO-assisted PTO Cleavage and Extension (L-PTOCE) composition”.

In one embodiment, when the CTO is unhybridized with the extended strand or the LPHO, the reporter molecule and the quencher molecule on the CTO are in close proximity to each other, thereby causing the quencher molecule to quench a signal from the reporter molecule.

In one embodiment, when the CTO is hybridized with the extended strand or the LPHO, the reporter molecule and the quencher molecule on the CTO are separated, thereby causing the quencher molecule to unquench a signal from the reporter molecule.

In one embodiment, (i) both the reporter molecule and the quencher molecule are linked to the capturing portion of the CTO, (ii) both the reporter molecule and the quencher molecule are linked to the templating portion of the CTO, or (iii) one of the reporter molecule and the quencher molecule is linked to the capturing portion of the CTO and the other is linked to the templating portion of the CTO.

In one embodiment, the LPHO is hybridized with complete or a partial sequence of the labeled portion of the CTO, and the reporter molecule and the quencher molecule on the CTO are separated, thereby causing the quencher molecule to unquench a signal from the reporter molecule.

In one embodiment, the LPHO comprises a nucleotide sequence which competes with the fragment for hybridization with the CTO.

In one embodiment, the LPHO comprises a nucleotide sequence which does not compete with the fragment for hybridization with the CTO.

In one embodiment, the composition for detecting the target nucleic acid reacts with the target nucleic acid to provide a signal dependent on the presence of the target nucleic acid. In particular, the composition provides a signal change as the target nucleic acid is amplified.

In one embodiment, the signal dependent on the presence of the target nucleic acid is a signal provided from the extended duplex.

In one embodiment, the reaction between the composition and the target nucleic acid may include an amplification reaction, and may include, for example, a signal ampli-fication reaction and/or a nucleic acid amplification reaction.

WO 2022-265463 discloses that various signal generation mechanisms known in the art for detecting a target nucleic acid have a signal-changing temperature range (SChTR) in which the signal changes depending on the presence of the target nucleic acid, and one or two signal-constant temperature ranges (SCoTRs) in which the signal is constant even in the presence of the target nucleic acid.

In addition, WO 2022-265463 discloses that various conventional signal generation mechanisms can be categorized into three types according to the number and/or order of these signal-changing temperature range and signal-constant temperature range:
(i) an Under-Signal-Change-type (UnderSC-type) signal generation mechanism having a melting characteristic that the signal-changing temperature range is lower than the signal-constant temperature range,
(ii) an Inter-Signal-Change-type (InterSC-type) signal generation mechanism having a melting characteristic that the signal-changing temperature range is higher than one of two signal-constant temperature ranges, and lower than the other of the two signal-constant temperature ranges, and
(iii) an Over-Signal-Change-type (OverSC-type) signal generation mechanism having a melting characteristic that the signal-changing temperature range is higher than the signal-constant temperature range.

Compositions for carrying out the three types of signal generation mechanisms can be classified into UnderSC-type compositions, InterSC-type compositions, and OverSC-type compositions, respectively.

The extended duplex generated by reaction between the composition for detecting a target nucleic acid according to the present disclosure and the target nucleic acid has a melting temperature (Tm) different from a Tm of the CTO/LPHO hybrid. By using the extended duplex and the CTO/LPHO hybrid having such different Tm values, the composition according to the present disclosure has a signal-changing temperature range (SChTR) in which the signal changes depending on the presence of the target nucleic acid, and two signal-constant temperature ranges (SCoTRs) in which the signal is constant even in the presence of the target nucleic acid.

Therefore, the L-PTOCE assay and composition according to the present disclosure can be classified into InterSC-type signal generation method and InterSC-type composition according to WO 2022-265463.

In one embodiment, the L-PTOCE composition has a signal-changing temperature range (SChTR) in which the signal changes depending on the presence of the target nucleic acid, and two signal-constant temperature range (SCoTR) in which the signal is constant even in the presence of the target nucleic acid,

In one embodiment, the signal-changing temperature range is higher than a first signal-constant temperature range of the two signal-constant temperature ranges, and lower than a second signal-constant temperature range of the two signal-constant temperature ranges.

In one embodiment, the extended duplex remains its double-stranded state and the CTO/LPHO hybrid dissociates into a single-stranded state at temperatures within the signal-changing temperature range in the presence of the target nucleic acid.

In one embodiment, both the extended duplex and the CTO/LPHO hybrid remain its double-stranded state at temperatures within the first signal-constant temperature range in the presence of the target nucleic acid.

In one embodiment, both the extended duplex and the CTO/LPHO hybrid dissociate into a single-stranded state at temperatures within the second signal-constant temperature range in the presence of the target nucleic acid.

In this regard, the L-PTOCE assay will be described in more detail with reference to the drawings as follows:

FIGS. 3 and 4 show the predominant conformation of oligonucleotides depending on the presence of the target nucleic acid and temperatures.

In the present disclosure, the extended duplex is not generated in the absence of the target nucleic acid or before reaction between the target nucleic acid and the composition for detecting the target nucleic acid. FIG. 3 (i) to FIG. 3 (iii) show, in the absence of the target nucleic acid or before reaction between the target nucleic acid and the L-PTOCE composition, the conformation of the CTO and the LPHO (i.e., the CTO/LPHO hybrid) within (i) the first signal-constant temperature range, (ii) the signal-changing temperature range, and (iii) the second signal-constant temperature range. Particularly, the CTO is hybridized with the LPHO at a temperature within the first signal-constant temperature range, and the reporter molecule and the quencher molecule on the CTO are separated, thereby causing the quencher molecule to unquench a signal from the reporter molecule (see FIG. 3 (i)). On the other hand, the CTO is not hybridized with the LPHO at a temperature within the signal-changing temperature range and the second signal-constant temperature range, and the reporter molecule and the quencher molecule on the CTO are in close proximity to each other, thereby causing the quencher molecule to quench a signal from the reporter molecule (see FIG. 3 (ii) and FIG. 3 (iii)).

Meanwhile, when a target nucleic acid is present, the L-PTOCE composition reacts with the target nucleic acid to generate the extended duplex. FIG. 4 (i) to FIG. 4 (iii) show, after reaction of the target nucleic acid and the L-PTOCE composition, the conformation of the CTO and the extended strand (i.e., extended duplex) within (i) the first signal-constant temperature range, (ii) the signal-changing temperature range, and (iii) the second signal-constant temperature range. Particularly, the CTO is hybridized with the extended strand at a temperature within the first signal-constant temperature range and the signal-changing temperature range, and the reporter molecule and the quencher molecule on the CTO are separated, thereby causing the quencher molecule to unquench a signal from the reporter molecule (see FIG. 4 (i) and FIG. 4 (ii)). On the other hand, the CTO is not hybridized with the extended strand at a temperature within the second signal-constant temperature range, and the reporter molecule and the quencher molecule on the CTO are in close proximity to each other, thereby causing the quencher molecule to quench a signal from the reporter molecule (see FIG. 4 (iii)). Here, there may be CTOs that do not participate in the reaction and LPHOs that do not participate in the reaction and are not cleaved (or separated). The LPHOs and CTOs that do not participate in the reaction may exist in the conformation of FIG. 3 (i) to FIG. 3 (iii).

Within the first signal-constant temperature range, where the target nucleic acid is absent (FIG. 3 (i)) or where the target nucleic acid is present (FIG. 4 (i)), the CTO is hybridized with the LPHO or the extended strand, and the reporter molecule and the quencher molecule on the CTO are separated, thereby causing the quencher molecule to unquench a signal from the reporter molecule. In other words, the L-PTOCE composition provides a constant signal even in the presence of the target nucleic acid within the first signal-constant temperature range.

Within the second signal-constant temperature range, where the target nucleic acid is absent (FIG. 3 (iii)) or where the target nucleic acid is present (FIG. 4 (iii)), the CTO is not hybridized with LPHO or the extended strand, and the reporter molecule and the quencher molecule on the CTO are in close proximity to each other, thereby causing the quencher molecule to quench a signal from the reporter molecule. In other words, the L-PTOCE composition provides a constant signal even in the presence of the target nucleic acid within the second signal-constant temperature range.

Within the signal-changing temperature range, where the target nucleic acid is absent (FIG. 3 (ii), the CTO is not hybridized with LPHO, and the reporter molecule and the quencher molecule on the CTO are in close proximity to each other, thereby causing the quencher molecule to quench a signal from the reporter molecule. On the other hand, within the signal-changing temperature range, where the target nucleic acid is present (FIG. 4 (ii)), the CTO is hybridized with the extended strand, and the reporter molecule and the quencher molecule on the CTO are separated, thereby causing the quencher molecule to unquench a signal from the reporter molecule. In other words, the L-PTOCE composition provides a signal change depending on the presence of the target nucleic acid within the signal-changing temperature range.

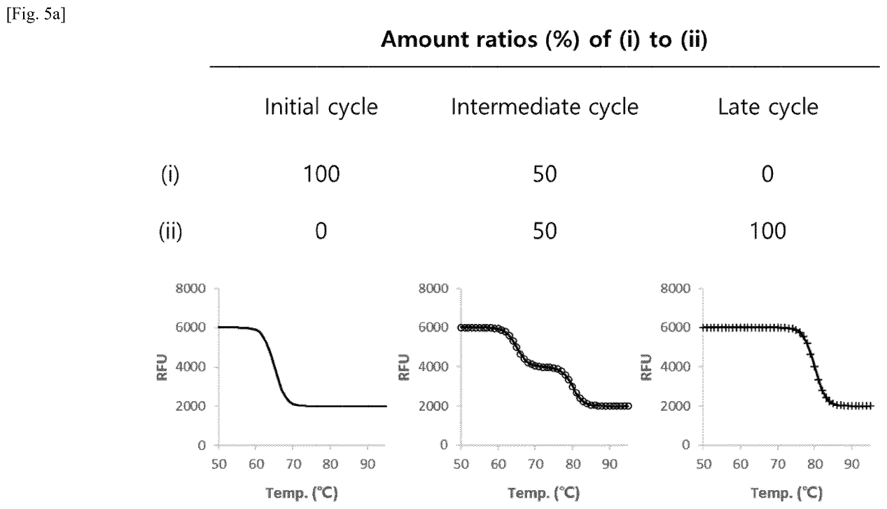

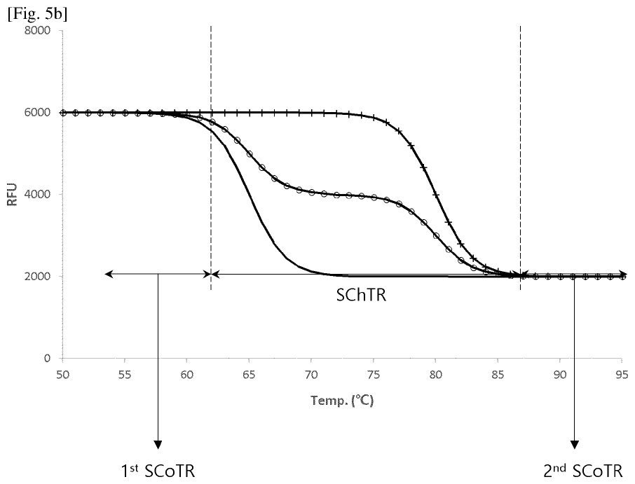

FIG. 5A shows the amount (or abundance) ratios of the CTO/LPHO hybrid and the extended duplex in the initial, intermediate, and late cycles of steps (a)-(e) of the L-PTOCE assay, along with their melt curves.

FIG. 5B represents a merged plot for three melt curves in FIG. 5A.

Specifically, in FIG. 5A, the numerical values in row (i), “100,” “50,” and “0,” represent the amount ratios of the CTO/LPHO, which may exist in any of the forms of FIG. 3 (i) to FIG. 3 (iii), and the numerical values in row (ii), “0,” “50,” and “100,” represent the amount ratios of the extended duplex, which may exist in any of the forms of FIG. 4 (i) to FIG. 4 (iii). The amount ratios of rows (i) and (ii) change as the cycle increases, i.e., as the target nucleic acid is amplified. In particular, a graph such as FIG. 5B can be obtained by merging the melting curves for the initial, intermediate, and late cycles.

As shown in FIG. 5B, the L-PTOCE composition according to the present disclosure has a signal-changing temperature range (SChTR) in which the signal changes depending on the presence of the target nucleic acid, and two signal-constant temperature ranges (SCoTR), the first and second signal-constant temperature ranges, in which the signal is constant even in the presence of the target nucleic acid. In addition, the signal-changing temperature range is higher than the first signal-constant temperature range, and lower than the second signal-constant temperature range.

The signal-changing temperature range and the signal-constant temperature range of the InterSC-type composition may be controlled by adjusting the Tm value of the extended duplex and the Tm value of the CTO/LPHO hybrid.

In one embodiment, the Tm of the extended duplex is adjustable by (i) a sequence and/or length of the fragment, (ii) a sequence and/or length of the CTO, or (iii) the sequence and/or length of the fragment and the sequence and/or length of the CTO.

In one embodiment, the Tm of the CTO/LPHO hybrid is adjustable by a sequence and/or length of the LPHO.

In one embodiment, the composition further comprises an enzyme having 5′ nuclease activity.

In certain embodiments, the enzyme having 5′ nuclease activity may be a DNA polymerase having 5′ nuclease activity.

In one embodiment, the composition further comprises an additional primer.

The composition as described herein may optionally include reagents required for performing target nucleic acid amplification reactions (e.g., PCR reactions) such as buffers, DNA polymerase cofactors, and deoxyribonucleotide-5-triphosphates. Optionally, the composition may also include various polynucleotide molecules, reverse transcriptase, various buffers and reagents, and antibodies that inhibit DNA polymerase activity. The composition may also include reagents necessary for performing positive and negative control reactions. Optimal amounts of reagents to be used in a given reaction can be readily determined by one of ordinary skill in the art having the benefit of the present disclosure. The above-described constituents of the composition may be present in separate containers, or a plurality of the constituents may be present in a single container.

### III. Method for Detecting Multiple Target Nucleic Acids Using L-PTOCE Assay

In a third aspect of the present disclosure, there is provided a method for detecting n target nucleic acids in a sample, comprising:
(a) detecting signals at n detection temperatures, while incubating with n compositions for detecting the n target nucleic acids, a sample suspected of containing at least one of the n target nucleic acids in a reaction vessel;
wherein n is an integer of 2 or more,
wherein the incubatiing comprises a plurality of reaction cycles and the detection of signals is carried out in at least one of the plurality of reaction cycles,
wherein each of the n compositions for detecting the n target nucleic acids provides a signal change at a corresponding detection temperature among the n detection temperatures in the presence of a corresponding target nucleic acid, the signal change indicating the presence of a corresponding target nucleic acid,
wherein a composition for detecting an i^{th} target nucleic acid among the n compositions for detecting the n target nucleic acids provides a signal change at an i^{th} detection temperature among the n detection temperatures and provides a constant signal at the other detection temperatures in the presence of the i^{th} target nucleic acid, the signal change indicating the presence of the i^{th} target nucleic acid,
wherein i represents an integer from 1 to n, and the i^{th} detection temperature is lower than a (i+1)^{th} detection temperature,
wherein within the temperature range covering all of the n detection temperatures, the composition for detecting the i^{th} target nucleic acid has a signal-changing temperature range (SChTR) in which the signal changes depending on the presence of the i^{th} target nucleic acid, and one or two signal-constant temperature ranges (SCoTRs) in which the signal is constant even in the presence of the i^{th} target nucleic acid,
wherein the composition for detecting the i^{th} target nucleic acid is any one of:
(i) an Under-Signal-Change-type (UnderSC-type) composition having a melting characteristic that the signal-changing temperature range is lower than the signal-constant temperature range,
(ii) an Inter-Signal-Change-type (InterSC-type) composition having a melting characteristic that the signal-changing temperature range is higher than one of two signal-constant temperature ranges, and lower than the other of the two signal-constant temperature ranges, and
(iii) an Over-Signal-Change-type (OverSC-type) composition having a melting characteristic that the signal-changing temperature range is higher than the signal-constant temperature range, and
wherein at least one of the n compositions for detecting n target nucleic acids is (ii) an InterSC-type composition which generates the signal according to the method as described above, and
(b) determining the presence of the n target nucleic acids from the signals detected in the step (a), wherein the presence of the i^{th} target nucleic acid is determined by the signal change detected at the i^{th} detection temperature.

Since the third aspect of the present disclosure uses the method of the first aspect of the present disclosure and the composition of the second aspect of the present disclosure described above, the common descriptions between them are omitted in order to avoid undue redundancy leading to the complexity of this specification.

The method for detecting n target nucleic acids according to the present disclosure uses (i) a plurality of L-PTOCE compositions, which are InterSC-type compositions and/or (ii) various combinations of the L-PTOCE composition with one or more of UnderSC-type, InterSC-type, and OverSC-type compositions, which are adopting various signal generation mechanisms known in the art for detecting target nucleic acids. The method of the present disclosure can detect multiple target nucleic acids using a single type of label and a single type of detector in single reaction vessel.

In particular, by adjusting signal-changing temperature ranges of the n compositions for detecting n target nucleic acids so that only a signal indicating the presence of a corresponding target nucleic acid is provided at each detection temperature, the presence of a particular target nucleic acid can be determined by a signal change measured at a particular detection temperature alone.

Hereinafter, the present disclosure will be described in detail as follows.

### Step (a): Incubation and Detection of Signal

First, signals are detected at n detection temperatures, while incubating with n compositions for detecting the n target nucleic acids, a sample suspected of containing at least one of the n target nucleic acids in a reaction vessel.

In one embodiment, the n target nucleic acids may include a nucleotide variation. For example, one of the n target nucleic acids may include one type of nucleotide variation, and another of the n target nucleic acids may include a different type of nucleotide variation.

The n target nucleic acids herein may be genes from n different organisms, n different genes from the same organism, or a combination thereof.

The incubating herein refers to any reaction that induces a signal change depending on the presence of a corresponding target nucleic acid at a corresponding detection temperature, as each of the target nucleic acids reacts with a corresponding composition for detecting a target nucleic acid.

In one embodiment, the incubating includes a plurality of cycles.

In one embodiment, the incubating may include an amplification reaction, and may include, for example, a signal amplification reaction and/or a nucleic acid ampli-fication reaction.

In one embodiment, the amplification reaction includes a plurality of cycles.

In one embodiment, the incubating is carried out under conditions that allow target amplification and a signal change by a composition for detecting a target nucleic acid. Such conditions include a temperature, salt concentration, and pH for the reaction.

In one embodiment, the incubating is carried out at a signal amplification process with no nucleic acid amplification.

In one embodiment, the signal may be amplified simultaneously with target ampli-fication. Alternatively, the signal may be amplified without target amplification.

In one embodiment, the signal change takes place during a process including signal amplification and target amplification.

In one embodiment, the amplification of the target nucleic acid may be carried out by a polymerase chain reaction (PCR). The PCR is widely used in the art to amplify target nucleic acids and includes cycles of denaturation of target nucleic acids, annealing (hybridization) between target nucleic acids and primers, and extension of primers (Mullis et al., U.S. Pat. Nos. 4,683,195, 4,683,202, and 4,800,159; Saiki et al., (1985) Science 230, 1350-1354).

Various DNA polymerase may be used in the amplification reaction and include the “Klenow” fragment of E. coli DNA polymerase I, thermostable DNA polymerase, and bacteriophage T7 DNA polymerase. In particular, the polymerase is a thermostable DNA polymerase obtainable from various bacteria, which include Thermus aquaticus (Taq), Thermus thermophilus (Tth), Thermus filiformis, Thermus flavus, Thermococcus litoralis, and Pyrococcus furiosus (Pfu). Most of the polymerases can be isolated from bacteria or are commercially available.

The above-described amplification method can amplify target nucleic acids and/or signals through a repetition of a series of reactions with or without change in temperature. The unit of amplification including the repetition of such series of reactions, is referred to as a “cycle”. The cycle may be expressed as the number of repetitions or a duration, depending on the amplification method used.

In one embodiment, the series of reactions may be carried out sequentially. For example, for a PCR, after denaturation of target nucleic acids (i.e., templates), annealing of primers and subsequently, extension of the primers may be carried out sequentially. In this case, the cycle may be expressed as the number of repetitions.

In one embodiment, the incubating may be carried out for a plurality of cycles that allows for the measurement of a signal change dependent on the presence of a target nucleic acid. For example, the plurality of cycles may include 2 to 100 cycles, 2 to 90 cycles, 2 to 80 cycles, 2 to 70 cycles, 2 to 60 cycles, 2 to 50 cycles, 2 to 40 cycles, 2 to 30 cycles, 2 to 20 cycles, 2 to 10 cycles, 5 to 100 cycles, 5 to 90 cycles, 5 to 80 cycles, 5 to 70 cycles, 5 to 60 cycles, 5 to 50 cycles, 5 to 40 cycles, 5 to 30 cycles, 5 to 20 cycles, 5 to 10 cycles, 10 to 100 cycles, 10 to 90 cycles, 10 to 80 cycles, 10 to 70 cycles, 10 to 60 cycles, 10 to 50 cycles, 10 to 40 cycles, 10 to 30 cycles, 10 to 20 cycles, 20 to 100 cycles, 20 to 90 cycles, 20 to 80 cycles, 20 to 70 cycles, 20 to 60 cycles, 20 to 50 cycles, 20 to 40 cycles, or 20 to 30 cycles, and particularly, may include 10 cycles, 15 cycles, 20 cycles, 25 cycles, 30 cycles, 35 cycles, 40 cycles, 45 cycles, or 50 cycles.

In one embodiment, the detection of signals may be carried out in each cycle, a selected few cycles, or an end-point cycle, of an incubation reaction including a plurality of cycles.

In one embodiment, the amplification reaction may be an amplification reaction for multiple target nucleic acids.

The term “amplification reaction for multiple target nucleic acids” as used herein refers to a reaction that amplifies two or more target nucleic acids in a single reaction vessel. The amplification reaction for multiple target nucleic acids refers to a reaction that amplifies two or more nucleic acids together. For example, the amplification reaction for multiple target nucleic acids may amplify, in a single reaction, 2 or more, 3 or more, 4 or more, 5 or more, 6 or more, 7 or more, 8 or more, 9 or more, 10 or more, 20 or more, 30 or more, 40 or more, or 50 or more target nucleic acids together.

In one embodiment, the method of the present invention may detect 2 to 50, 2 to 40, 2 to 30, 2 to 20, 2 to 15, 2 to 12, 2 to 10, 2 to 9, 2 to 8, 2 to 7, 2 to 6, 2 to 5, 3 to 50, 3 to 40, 3 to 30, 3 to 20, 3 to 15, 3 to 12, 3 to 10, 3 to 9, 3 to 8, 3 to 7, 3 to 6, 3 to 5, 4 to 50, 4 to 40, 4 to 30, 4 to 20, 4 to 15, 4 to 12, 4 to 10, 4 to 9, 4 to 8, 4 to 7, 4 to 6, or 4 to 5 target nucleic acids by using a single type of label in a single reaction vessel.

The method according to the present disclosure is used to determine whether at least one of n target nucleic acids is present in a sample. For example, when n is 2, the method of the present disclosure may be used to determine whether at least one of a first target nucleic acid and a second target nucleic acid is present in a sample. As another example, when n is 3, the method of the present disclosure may be used to determine whether at least one among a first target nucleic acid, a second target nucleic acid, and a third target nucleic acid is present in a sample.

In one embodiment, n is an integer of 2 or more. For example, n may be 2, 3, 4, 5, 6, 7, 8, 9, 10, 11, 12, 13, 14, 15, 20, 25, 30, 35, 40, 45, or 50, but is not limited thereto.

In the present disclosure, a combination of compositions for detecting n target nucleic acids is used to detect each of n target nucleic acids, wherein n is an integer of 2 or more. In other words, a combination of compositions for detecting the first to the n^{th} target nucleic acid is used to detect the first target nucleic acid to the n^{th} target nucleic acid.

As used herein, the term “combination of compositions for detecting the first to the n^{th} target nucleic acids” refers to a compilation or mixture of compositions specific for each of the first to n^{th} target nucleic acids. Herein, a composition for detecting one target nucleic acid is specific for the same target nucleic acid. The expression “a composition for detecting a target nucleic acid is specific for the same target nucleic acid” means that the composition for detecting the target nucleic acid is involved in the detection of the same target nucleic acid, but is not involved in the detection of other target nucleic acids. In other words, the expression means that the composition for detecting the target nucleic acid interacts with the same target nucleic acid but does not interact with other target nucleic acids.

Herein, the composition for detecting the n^{th} target nucleic acid is specific for the same target nucleic acid. For example, the composition for detecting the first target nucleic acid is specific for the first target nucleic acid, the composition for detecting the second target nucleic acid is specific for the second target nucleic acid, and the composition for detecting the third target nucleic acid is specific to the third target nucleic acid.

The combination of compositions for detecting the first to the n^{th} target nucleic acids as used herein is employed in a single reaction. In other words, the compositions for detecting the first to the n^{th} target nucleic acids coexist in a single reaction solution or reaction vessel.

As used herein, the term “composition for detecting a target nucleic acid” refers to a composition comprising components used to detect a target nucleic acid.

According to the method of the present disclosure, each of the compositions for detecting the first to n^{th} target nucleic acids comprises a label that provides a signal depending on the presence of the same target nucleic acid, and the signals provided from each of the compositions for detecting the first to n^{th} target nucleic acids are not distinguished from each other by a single detection channel.

The composition for detecting a target nucleic acid may comprises various oligonu-cleotides involved in amplifying and/or detecting the target nucleic acid.

The label herein may be linked to an oligonucleotide or may exist in free form. Alternatively, the label may be incorporated into the oligonucleotide during the incubating.

Although the label and oligonucleotide are described as key elements in the compositions for detecting the first to n^{th} target nucleic acids, it is to be understood by one of skill in the art that various other components may be further included in the compositions.

Examples of components included in the compositions for detecting the target nucleic acid include, but are not limited to, an oligonucleotide set used to amplify or detect the target nucleic acid, a label, a nucleic acid polymerase, a buffer, a polymerase cofactor, and a deoxyribonucleotide-5-triphosphate. Optionally, the compositions may also include various polynucleotide molecules, reverse transcriptase, various buffers and reagents, and antibodies that inhibit DNA polymerase activity. The compositions may also include reagents necessary for performing positive and negative control reactions. Optimal amounts of reagents to be used in a given reaction can be readily determined by one of ordinary skill in the art having the benefit of the present disclosure. The above-described constituents of the composition may be present in separate containers, or a plurality of the constituents may be present in a single container.

Each of n compositions for detecting n target nucleic acids used in the present disclosure is any one of (i) an Under-Signal-Change-type (UnderSC-type) composition, (ii) an Inter-Signal-Change-type (InterSC-type) composition, and (iii) an Over-Signal-Change-type (OverSC-type) composition, provided that at least one of the n compositions for detecting n target nucleic acids is an InterSC-type composition which generates the signal according to the L-PTOCE assay as described above. For example, when n is 2, at least one of the composition for detecting the first target nucleic acid and the composition for detecting the second target nucleic acid is a L-PTOCE composition. Particularly, when n is 2, exemplary combinations of the composition for detecting the first target nucleic acid and the composition for detecting the second target nucleic acid are as shown in Table 1 below. When n is 3, exemplary combinations of the compositions for detecting the first to third target nucleic acids are as shown in Table 2.

In Tables 1 and 2, “UnderSC,” “InterSC,” and “OverSC” refer to UnderSC-type, InterSC-type, and OverSC-type compositions, respectively.

TABLE 1

| | Composition for | Composition for |
|---|---|---|
| | detecting the first | detecting the second |
| n = 2 | target nucleic acid | target nucleic acid |
| 1 | UnderSC | L-PTOCE |
| 2 | L-PTOCE | L-PTOCE |
| 3 | L-PTOCE | InterSC |
| 4 | InterSC | L-PTOCE |
| 5 | L-PTOCE | OverSC |

TABLE 2

| | Composition for | Composition for | Composition for |
|---|---|---|---|
| | detecting the first | detecting the second | detecting the third |
| n = 3 | target nucleic acid | target nucleic acid | target nucleic acid |
| 1 | L-PTOCE | InterSC | InterSC |
| 2 | L-PTOCE | InterSC | OverSC |
| 3 | UnderSC | L-PTOCE | InterSC |
| 4 | UnderSC | L-PTOCE | OverSC |
| 5 | InterSC | L-PTOCE | InterSC |
| 6 | InterSC | L-PTOCE | OverSC |
| 7 | UnderSC | InterSC | L-PTOCE |
| 8 | InterSC | InterSC | L-PTOCE |
| 9 | L-PTOCE | L-PTOCE | InterSC |
| 10 | L-PTOCE | L-PTOCE | OverSC |
| 11 | L-PTOCE | InterSC | L-PTOCE |
| 12 | UnderSC | L-PTOCE | L-PTOCE |
| 13 | InterSC | L-PTOCE | L-PTOCE |
| 14 | L-PTOCE | L-PTOCE | L-PTOCE |

Details of the UnderSC-type, InterSC-type, and OverSC-type compositions are found in WO2022-265463, which is incorporated herein by reference in its entirety.

In one embodiment, each of the n compositions for detecting the target nucleic acids provides a signal change at a corresponding detection temperature among the n detection temperatures, the signal change indicating the presence of a corresponding target nucleic acid.

For example, the composition for detecting an i^{th} target nucleic acid among the n target nucleic acids provides a signal change at an i^{th} detection temperature among the n detection temperatures and provides a constant signal at the other detection temperatures in the presence of the i^{th} target nucleic acid.

In one embodiment, i represents an integer from 1 to n, and the i^{th} detection temperature is lower than the (i+1)^{th} detection temperature. In one embodiment, when i is n, there is no i+1 detection temperature (i.e., n+1 detection temperature). For example, when n is 3, i represents an integer from 1 to 3, and there are a first detection temperature, a second detection temperature, and a third detection temperature, wherein the first detection temperature is lower than the second detection temperature, and the second detection temperature is lower than the third detection temperature.

According to the present disclosure, within the temperature range covering all of the n detection temperatures, the composition for detecting the i^{th} target nucleic acid has a signal-changing temperature range (SChTR) in which the signal changes depending on the presence of the i^{th} target nucleic acid, and one or two signal-constant temperature ranges (SCoTRs) in which the signal is constant even in the presence of the i^{th} target nucleic acid.

In one embodiment, the composition for detecting the i^{th} target nucleic acid is any one of: (i) an Under-Signal-Change-type (UnderSC-type) composition having a melting characteristic that the signal-changing temperature range is lower than the signal-constant temperature range, (ii) an Inter-Signal-Change-type (InterSC-type) composition having a melting characteristic that the signal-changing temperature range is higher than one of two signal-constant temperature ranges, and lower than the other of the two signal-constant temperature ranges, and (iii) an Over-Signal-Change-type (OverSC-type) composition having a melting characteristic that the signal-changing temperature range is higher than the signal-constant temperature range.

In one embodiment, the composition for detecting the i^{th} target nucleic acid, in the presence of the i^{th} target nucleic acid, provides a signal change upon amplification of the target nucleic acid at the i^{th} detection temperature (i.e., change in an i^{th} signal) while providing no signal change at the other detection temperatures, even as the target nucleic acid is amplified (i.e., the signal is constant). That is, the composition for detecting the i^{th} target nucleic acid have a signal-changing temperature range in which the signal changes as the i^{th} target nucleic acid is amplified, and signal-constant temperature range(s) in which the signal is constant even as the i^{th} target nucleic acid is amplified.

The term “i^{th} signal” as used herein refers to a signal provided at an i^{th} detection temperature by the composition for detecting an i^{th} target nucleic acid, which is interchangeably used with “signal at an i^{th} detection temperature”.

In one embodiment, when detecting n target nucleic acids, the i^{th} signal may mean a signal provided by the n compositions for detecting target nucleic acids including the composition for detecting the i^{th} target nucleic acid, at the i^{th} detection temperature.

In one embodiment, the composition for detecting the i^{th} target nucleic acid, in the absence of the i^{th} target nucleic acid, provides no signal change, i.e., provides a constant signal at the i^{th} detection temperature during an incubation reaction (e.g., a target nucleic acid amplification reaction).

In one embodiment, the signal-changing temperature range is a temperature range in which a difference between signal values (e.g., signal intensities) in the presence of the target nucleic acid and in the absence of the target nucleic acid is generated.

In one embodiment, the signal-changing temperature range is a temperature range in which the signal value changes depending on the level of amplification of the target nucleic acid (e.g., the amount of the amplified target nucleic acid).

In one embodiment, the signal-constant temperature range is a temperature range in which the signal value does not change regardless of the presence of the target nucleic acid. In other words, the signal-constant temperature range is a temperature range in which there is no difference between the signal value in the presence of the target nucleic acid and the signal value in the absence of the target nucleic acid.

In one embodiment, the i^{th} detection temperature may be selected from within the signal-changing temperature range of the composition for detecting the i^{th} target nucleic acid. In the present disclosure, the composition for detecting the i^{th} target nucleic acid is referred to as having the i^{th} detection temperature. In addition, the i^{th} target nucleic acid corresponding to the composition for detecting the i^{th} target nucleic acid may be referred to as a target nucleic acid having the i^{th} detection temperature.

In one embodiment, one detection temperature, which is determined by the composition for detecting a corresponding target nucleic acid, is assigned to one target nucleic acid.

In certain embodiments, when n is 2, the composition for detecting the first target nucleic acid provides a signal change at the first detection temperature and provides a constant signal at the second detection temperature in the presence of the first target nucleic acid; and the composition for detecting the second target nucleic acid provides a signal change at the second detection temperature and provides a constant signal at the first detection temperature in the presence of the second target nucleic acid.

In certain embodiments, when n is 3, the composition for detecting the first target nucleic acid provides a signal change at the first detection temperature and provides a constant signal at the second detection temperature and the third detection temperature in the presence of the first target nucleic acid; the composition for detecting the second target nucleic acid provides a signal change at the second detection temperature and provides a constant signal at the first detection temperature and the third detection temperature in the presence of the second target nucleic acid; and the composition for detecting the third target nucleic acid provides a signal change at the third detection temperature and provides a constant signal at the first detection temperature and the second detection temperature in the presence of the third target nucleic acid.

In one embodiment, the i^{th} detection temperature is selected from within the signal-changing temperature range of the composition for detecting the i^{th} target nucleic acid, and the i^{th} detection temperature is not included in the signal-changing temperature ranges of the compositions for detecting the other target nucleic acids.

In one embodiment, the signal-changing temperature range of any one of the compositions for detecting target nucleic acids may overlap with the signal-changing temperature range of a composition for detecting a target nucleic acid having an adjacent detection temperature, while not overlapping with the signal-changing temperature range of a composition for detecting a target nucleic acid having a detection temperature not adjacent thereto. In this case, the detection temperature of the composition for detecting a target nucleic acid that has the signal-changing temperature range overlapping with the signal-changing temperature range of the composition for detecting another target nucleic acid is selected within the signal-changing temperature range that does not overlap with the signal-changing temperature range of the composition for detecting another target nucleic acid. By selecting the detection temperature as such, only the signal indicative of the presence of a single particular target nucleic acid can be provided at a single detection temperature

In one embodiment, the signal-changing temperature range of any one of compositions for detecting target nucleic acids may overlap with the signal-changing temperature range of the composition for detecting a target nucleic acid having an adjacent detection temperature, but either of the two signal-changing temperature ranges is not completely included in the other signal-changing temperature range.

The term “adjacent detection temperature” is used herein to refer to consecutive detection temperatures among n detection temperatures, and for example, the adjacent detection temperature of the i^{th} detection temperature is the (i−1)^{th} detection temperature or the (i+1)^{th} detection temperature.

In one embodiment, the signal-changing temperature range of the composition for detecting the i^{th} target nucleic acid may partially overlap with the signal-changing temperature range of the composition for detecting a target nucleic acid having an adjacent detection temperature, while not overlapping with the signal-changing temperature range of the composition for detecting a target nucleic acid having a detection temperature not adjacent thereto.

In one embodiment, the composition for detecting the i^{th} target nucleic acid may have one signal-changing temperature range and one signal-constant temperature range.

In one embodiment, the composition for detecting the i^{th} target nucleic acid may have one signal-changing temperature range and two signal-constant temperature ranges.

In one embodiment, the composition for detecting the i^{th} target nucleic acid comprises a label that provides a signal dependent on the presence of the i^{th} target nucleic acid.

In one embodiment, the label herein may be linked to an oligonucleotide or may exist in free form. Alternatively, the label may be incorporated into the oligonucleotide during the incubating (e.g., a nucleic acid amplification). In other words, the composition for detecting the target nucleic acid may initially include a labeled oligonu-cleotide or may provide a labeled oligonucleotide as the label is incorporated into a newly generated oligonucleotide (e.g., an extended strand) during an incubation reaction.

In one embodiment, the composition for detecting the i^{th} target nucleic acid includes an incorporating label that is incorporated into an oligonucleotide during the in-cubating and provides a signal depending on the presence of the i^{th} target nucleic acid.

In one embodiment, the composition for detecting the i^{th} target nucleic acid provides a labeled oligonucleotide that serves to provide a signal depending on the presence of the i^{th} target nucleic acid.

In one embodiment, the composition for detecting the i^{th} target nucleic acid initially includes a labeled oligonucleotide that serves to provide a signal depending on the presence of the i^{th} target nucleic acid. The CTO as described herein corresponds to an example of the labeled oligonucleotides.

Alternatively, the composition for detecting the i^{th} target nucleic acid may include an oligonucleotide and a label that provides a signal depending on the presence of the i^{th} target nucleic acid, and the label is incorporated into the oligonucleotide during an incubation reaction (e.g., a nucleic acid amplification reaction), thereby providing a labeled oligonucleotide that serves to provide a signal depending on the presence of the i^{th} target nucleic acid.

As used herein, the term “labeled oligonucleotide” refers to an oligonucleotide involved in the generation of a signal being detected.

In one embodiment, the labeled oligonucleotide may comprise an oligonucleotide that specifically hybridizes to a target nucleic acid (e.g., a probe or a primer); when the probe or primer hybridized to the target nucleic acid is cleaved to release a fragment, the labeled oligonucleotide may comprise a capture oligonucleotide that specifically hybridizes to the fragment; when the fragment hybridized to the capture oligonu-cleotide is extended to form an extended strand, the labeled oligonucleotide may comprise an oligonucleotide that specifically hybridizes to the extended strand, an oligonucleotide that is produced by incorporating a label during the fragment extension, an oligonucleotide that specifically hybridizes to the capture oligonu-cleotide, and a combination thereof.

In one embodiment, the labeled oligonucleotide includes an oligonucleotide involved in actual signal generation. For example, hybridization or non-hybridization between the labeled oligonucleotide and another oligonucleotide (e.g., an oligonucleotide comprising a nucleotide sequence complementary to the labeled oligonucleotide or the target nucleic acid) determines signal generation.

In one embodiment, the labeled oligonucleotide may be a ‘probe’ known in the art. The term “probe” as used herein refers to a single-stranded nucleic acid molecule comprising one or more portions substantially complementary to a target nucleic acid sequence. According to an embodiment of the present disclosure, the 3′-end of the probe is “blocked” to prohibit its extension. The blocking may be achieved in accordance with conventional methods. For instance, the blocking may be performed by adding to the 3′-hydroxyl group of the last nucleotide a chemical moiety such as biotin, labels, phosphate groups, alkyl groups, non-nucleotide linkers, phosphorothioate or alkane-diol residues. Alternatively, the blocking may be carried out by removing the 3′-hydroxyl group of the last nucleotide or using a nucleotide with no 3′-hydroxyl group such as dideoxynucleotide.

In one embodiment, the labeled oligonucleotide may be composed of at least one oligonucleotide. According to an embodiment of the present invention, when the labeled oligonucleotide is composed of a plurality of oligonucleotides, the labeled oligonucleotide may be labeled in various fashions. For example, all or portion of the plurality of oligonucleotides may have at least one label.

In one embodiment, the label may be a single label or interactive labels.

For example, the single label includes a fluorescent label, a luminescent label, a chemiluminescent label, an electrochemical label, and a metal label. In one embodiment, the single label provides different signals (for example, different signal intensities) depending on its presence on a double strand or a single strand. In one embodiment, the single label is a fluorescent label. Preferred types and binding sites of single fluorescent labels used in the present disclosure are disclosed in U.S. Pat. Nos. 7,537,886 and 7,348,141, the teachings of which are incorporated herein by reference in their entirety. For example, the single fluorescent label includes, without limitation, JOE, FAM, TAMRA, ROX, and fluorescein-based label. The single label may be linked to an oligonucleotide by various methods. For example, the label may be linked to a probe via a spacer containing carbon atoms (e.g., a 3-carbon spacer, a 6-carbon spacer, or a 12-carbon spacer).

In one embodiment, the interactive labels may include at least one reporter molecule and at least one quencher molecule. In particular, the interactive labels may include one reporter molecule and one quencher molecule. Or, the interactive labels may include one reporter molecule and two quencher molecules.

Reporter molecule and quencher molecule useful in the present disclosure may include any molecules known in the art, details of which are found in the section describing reporter molecules and quencher molecules in the first aspect of the specification.

In one embodiment, when the label is interactive labels, the interactive labels may include at least one reporter molecule and at least one quencher molecule, wherein the interactive labels may be all linked to one oligonucleotide or may be linked to each of a plurality of oligonucleotides.

In one embodiment, an incorporating label may be used in the process of incorporating a label during a primer extension to generate a signal (e.g., Plexor technology, Sherrill C B et al., Journal of the American Chemical Society, 126:4550-45569 (2004)). In addition, the incorporating label may be used in a signal generation by a duplex formed in a manner dependent on cleavage of a mediation oligonucleotide hybridized to a target nucleic acid.

In one embodiment, the incorporating label may be generally linked to a nucleotide. In addition, a nucleotide having a non-natural base may be used.

As used herein, the term “non-natural base” refers to derivatives of natural bases such as adenine (A), guanine (G), thymine (T), cytosine (C) and uracil (U), which are capable of forming hydrogen-bonding base pairs. The term “non-natural base” as used herein includes bases having base pairing patterns different from natural bases as mother compounds, as described, for example, in U.S. Pat. Nos. 5,432,272, 5,965,364, 6,001,983, and 6,037,120. The base pairing between non-natural bases includes two or three hydrogen bonds as with natural bases. The base pairing between non-natural bases is also formed in a specific manner. Specific examples of non-natural bases include the following bases in base pair combinations: iso-C/iso-G, iso-dC/iso-dG, Z/P, V/J, K/X, H/J, Pa/Ds, Pa/Q, Pn/Ds, Pn/Dss, Px/Ds, NaM/5SICS, 5FM/5SICS, and M/N (see U.S. Pat. Nos. 5,432,272; 5,965,364; 6,001,983; 6,037,120; 6,140,496; 6,627,456; 6,617,106; and 7,422,850; and Filip Wojciechowski et al., Chem. Soc. Rev., 2011, 40, 5669-5679).

Conventional methods for detecting multiple target nucleic acids have disadvantages of requiring the use of different types of fluorescent labels for real-time detection of multiple target nucleic acids, or requiring an additional analysis, such as melting curve analysis, even when using a single type of fluorescent label. In contrast, the method according to the present disclosure can detect multiple target nucleic acids in a real-time manner using a single type of label (e.g., a single fluorescent label) without an additional analysis such as melting analysis.

In one embodiment, each of the n compositions for detecting target nucleic acids provides one or more duplexes.

As used herein, the term “duplex” refers to a double-stranded nucleic acid molecule formed by hybridizing two single-stranded nucleic acid molecules having a sequence partially or totally complementary to each other under hybridization conditions. The two single-stranded nucleic acid molecules forming the duplex may exist in associated form (i.e., a double-stranded molecule) or dissociated form (i.e., two single-stranded molecules) depending on the temperature (in particular, the detection temperature). In this respect, the term “duplex” when referring the expression “a composition for detecting a target nucleic acid provides a duplex” is used to encompass both a duplex in associated form and a duplex in dissociated form.

In one embodiment, the duplex may be also referred to as “hybrid”.

The expression “a composition for detecting a target nucleic acid provides a duplex” as used herein may mean that the composition provides a duplex in associated form and/or a duplex in dissociated form. Likewise, the expression “a composition for detecting a target nucleic acid generates a duplex during incubating” as used herein may mean that the composition generates a duplex in associated form and/or a duplex in dissociated form during an incubation reaction.

In one embodiment, at least one of the duplexes provided by the composition for detecting a target nucleic acid is a duplex providing a signal. In particular, the duplex is a duplex providing a signal change. In other words, the composition for detecting an i^{th} target nucleic acid provides a duplex providing a signal, and particularly, the composition for detecting the i^{th} target nucleic acid provides a duplex providing a signal change depending on the presence of the i^{th} target nucleic acid.

The term “duplex providing a signal” as used herein refers to a duplex capable of providing a signal that can be distinguished depending on whether the duplex is in associated form or dissociated form. For example, this means that the duplex in associated form generates (or extinguishes) a signal, and the duplex in dissociated form extinguishes (or generates) a signal.

In one embodiment, the duplex providing the signal may include at least one label.

As used herein, the term “duplex providing a signal change” refers to a duplex providing a signal change indicative of the presence of a target nucleic acid as the amount of the duplex providing the signal change changes depending on the presence of the target nucleic acid. In particular, the extended duplex according to the present disclosure corresponds to an example of a duplex providing a signal change described herein.

In one embodiment, the duplex providing the signal change includes a label. In particular, at least one label is linked to at least one of the two single-stranded nucleic acid molecules constituting the duplex. For example, the duplex providing the signal change includes a single label, and in this case, the single label is linked to any one of the two single-stranded nucleic acid molecules constituting the duplex. As another example, the duplex providing the signal change includes interactive labels, and in this case, the interactive labels are all linked to one of the two single-stranded nucleic acid molecules constituting the duplex providing the signal change, or one of the interactive labels is linked to one of the two single-stranded nucleic acid molecules and the other of the interactive label is linked to the other of the two single-stranded nucleic acid molecules.

In one embodiment, the composition for detecting the i^{th} target nucleic acid provides a duplex providing a signal change.

In one embodiment, the composition for detecting the i^{th} target nucleic acid provides a signal from the label when the duplex providing the signal change is in associated form. In other words, the composition for detecting the i^{th} target nucleic acid provides a signal depending on association of the two single-stranded nucleic acid molecules constituting the duplex.

In an alternative embodiment, the composition for detecting the i^{th} target nucleic acid provides a signal from the label when the duplex providing the signal change is in dis-sociated form. In other words, the composition for detecting the i^{th} target nucleic acid provides a signal depending on dissociation of the two single-stranded nucleic acid molecules constituting the duplex.

In one embodiment, the association or dissociation of the duplex may depend on temperature.

In one embodiment, the duplex providing the signal change may be a duplex that has initially (originally) been included in the composition for detecting a target nucleic acid.

In one embodiment, when the duplex providing the signal change has been included in the composition for detecting the target nucleic acid, the duplex may be generated by hybridization between a labeled oligonucleotide and an oligonucleotide hybridizable with the labeled oligonucleotide. The CTO/LPHO hybrid of the present disclosure or a double-stranded nucleic acid hybridization probe (U.S. Pat. No. 7,799,522), also known as Yin-Yang probe, is an exemplary duplex providing a signal change depending on the presence of a target nucleic acid, which has initially been included in the composition for detecting the target nucleic acid.

In one embodiment, when the duplex providing the signal change has initially been included in the composition for detecting the target nucleic acid, the amount of the duplex providing the signal change varies, in particular, decreases, in a manner dependent on the presence of the target nucleic acid, thereby providing the signal change. For example, the amount of the CTO/LPHO hybrid decreases as extended duplexes are generated depending on the presence of target nucleic acid, thus providing a signal change depending on the presence of the target nucleic acid.

In one embodiment, the duplex providing the signal change may be a duplex newly provided by the composition for detecting a target nucleic acid during an incubation reaction.

In one embodiment, the duplex providing the signal change, which is generated during the incubation reaction, may be provided by hybridization between a labeled oligonucleotide and the target nucleic acid.

Signals by formation of a duplex between the labeled oligonucleotide and the target nucleic acid may be generated by various methods, including Scorpion method (Whitcombe et al., Nature Biotechnology 17:804-807 (1999)), Sunrise (or Amplifluor) method (Nazarenko et al., Nucleic Acids Research, 25(12):2516-2521 (1997), and U.S. Pat. No. 6,117,635), LUX method (U.S. Pat. No. 7,537,886), Plexor method (Sherrill C B, et al., Journal of the American Chemical Society, 126:4550-4556 (2004)), Molecular beacon method (Tyagi et al., Nature Biotechnology v.14 Mar. 1996), Hybeacon method (French D J et al., Mol. Cell Probes, 15(6):363-374 (2001)), adjacent hybridization probe method (Bernard P.S. et al., Anal. Biochem., 273:221 (1999)), and LNA method (U.S. Pat. No. 6,977,295).

In one embodiment, the duplex providing the signal change, which is generated during the incubation reaction, may be a duplex generated by a cleavage reaction dependent on the presence of the target nucleic acid. The extended duplex according to the present disclosure is an example of a duplex generated by a cleavage reaction dependent on the presence of the target nucleic acid.

In one embodiment, the signal change is generated by a duplex generated in a manner dependent on the cleavage of a mediation oligonucleotide specifically hybridized to the target nucleic acid.

As used herein, the term “mediation oligonucleotide” refers to an oligonucleotide mediating the generation of a duplex, not including a target nucleic acid.

In one embodiment, the cleavage of the mediation oligonucleotide alone does not generate a signal, but after hybridization and cleavage of the mediation oligonu-cleotide, a fragment (a cleavage product) produced by the cleavage is involved in a series of reactions for signal generation.

In one embodiment, the hybridization or cleavage of the mediation oligonucleotide alone does not generate a signal.

In one embodiment, the mediation oligonucleotide includes an oligonucleotide that hybridizes with a target nucleic acid and is cleaved to release a fragment, thereby mediating the generation of a duplex.

In one embodiment, the fragment mediates the generation of a duplex by extension of the fragment on a capture oligonucleotide.

According to an embodiment, the mediation oligonucleotide comprises (i) a targeting portion comprising a hybridizing nucleotide sequence with a target nucleic acid, and (ii) a tagging portion comprising a non-hybridizing nucleotide sequence with the target nucleic acid.

In one embodiment, the composition for detecting a target nucleic acid may include a tagging oligonucleotide that hybridizes with the target nucleic acid, and the cleavage reaction dependent on the presence of the target nucleic acid may involve cleavage of the tagging oligonucleotide. The tagging oligonucleotide corresponds to an example of the mediation oligonucleotide described above, and the PTO of the present disclosure corresponds to an example of the tagging oligonucleotide

According to an embodiment, cleavage of the mediation oligonucleotide releases a fragment, and the fragment is specifically hybridized to a capture oligonucleotide and extended on the capture oligonucleotide. When the capture oligonucleotide comprises a label, the capture oligonucleotide corresponds to an example of the labeled oligonu-cleotide described herein.

According to an embodiment, the mediation oligonucleotide hybridized with a target nucleic acid is cleaved and releases a fragment, the fragment is specifically hybridized to a capture oligonucleotide, and the fragment is extended to generate an extended strand, which induces the formation of an extended duplex between the extended strand and the capture oligonucleotide, thereby providing a signal indicative of the presence of the target nucleic acid.

According to an embodiment, a third oligonucleotide comprising a hybridizing nucleotide sequence with the extended strand may be used. When the third oligonu-cleotide is used, the hybridization of the extended strand and the third oligonucleotide forms another type of duplex, thereby providing a signal indicating the presence of the target nucleic acid (e.g., PCE-SH). In this case, another type of duplex is a duplex providing the signal change.

Signals by a duplex generated in a manner dependent on cleavage of the mediation oligonucleotide may be generated by various methods, including PTOCE (PTO cleavage and extension) method (WO 2012/096523), PCE-SH (PTO Cleavage and Extension-Dependent Signaling Oligonucleotide Hybridization) method (WO 2013/115442), and PCE-NH (PTO Cleavage and Extension-Dependent Non-Hybridization) method (WO 2014/104818).

Regarding the terms disclosed in the above references, the corresponding examples of the oligonucleotides are as follows: the mediation oligonucleotide corresponds to a PTO (Probing and Tagging Oligonucleotide), the capture oligonucleotide corresponds to a CTO (Capturing and Templating Oligonucleotide), and the third oligonucleotide corresponds to an SO (Signaling Oligonucleotide) or an HO (Hybridization Oligonu-cleotide). The SO, HO, CTO, extended strand, or a combination thereof may play the role of a labeled oligonucleotide.

In one embodiment, the duplex providing the signal change may be a single-typed duplex or plural-typed duplexes. Specifically, when the duplex providing the signal change is a single-typed duplex, the number of the duplexes may be 1, and when the duplex providing the signal change is plural-typed duplexes, the number of the duplexes may be 2, 3, 4, 5, 6, 7, 8, 9, 10, 15, or 20, specifically, 2, 3, or 4, and more specifically, 2 or 3.

In one embodiment, the single-typed duplex, or any of the plural-typed duplexes includes a label.

In one embodiment, when the duplex providing the signal change is a single-typed duplex, the amount of the single-typed duplex changes depending on the presence of a target nucleic acid, thereby changing the signal.

In one embodiment, when the duplex is plural-typed duplexes, the amount ratios between the plural-typed duplexes changes depending on the presence of a target nucleic acid, thereby changing the signal.

In one embodiment, Tm values of the plural-typed duplexes are different from each other. For example, the Tm values of the duplexes are different from each other by at least 2° C., at least 3° C., at least 4° C., at least 5° C., at least 7° C., at least 8° C., at least 9° C., at least 10° C., at least 11° C., at least 12° C., at least 13° C., at least 14° C., at least 15° C., or at least 20° C.

In one embodiment, the amount of the duplex refers to the sum of the amount of a duplex in dissociated form and the amount of a duplex in associated form.

In one embodiment, at least two of the plural-typed duplexes include the same single-stranded nucleic acid molecule. When the plural-typed duplexes include the same single-stranded nucleic acid molecule, the same single-stranded nucleic acid molecule is included in a first duplex initially included in the composition for detecting the target nucleic acid, and during an incubation reaction, a new second duplex including the same single-stranded nucleic acid molecule may be generated. In this case, the same single-stranded nucleic acid molecule included in the first duplex can be considered to be consumed by participating in the generation of the second duplex during the incubation reaction, resulting in decreased amount of the first duplex and increased amount of the second duplex.

In one embodiment, the signal-changing temperature range of the composition for detecting the i^{th} target nucleic acid may be determined on the basis of the length and/or sequence of the duplex providing a signal change.

In one embodiment, the composition for detecting the i^{th} target nucleic acid provides a single-typed duplex, the composition for detecting the i^{th} target nucleic acid may have one signal-changing temperature range and one signal-constant temperature range. The signal-changing temperature range and signal-constant temperature range may be de-termined on the basis of the length and/or sequence of the single-typed duplex.

In one embodiment, when the composition for detecting the i^{th} target nucleic acid provides plural-typed duplexes, in particular, two different types of duplexes, the composition for detecting the i^{th} target nucleic acid may have one signal-changing temperature range and two signal-constant temperature ranges. The signal-changing temperature range and the signal-constant temperature ranges may be determined on the basis of the lengths and/or sequences of the two different types of duplexes.

In one embodiment, any one of the n compositions for detecting the target nucleic acids may include an amplification oligonucleotide that serves to amplify a corresponding target nucleic acid. In one embodiment, the amplification oligonucleotide may be the same as the labeled oligonucleotide.

As used herein, the term “amplification oligonucleotide” refers to any oligonu-cleotides that serve to amplify target nucleic acids.

In one embodiment, the amplification oligonucleotide may be a ‘primer’ known in the art. As used herein, the term ‘primer’ refers to an oligonucleotide that is capable of acting as a point of initiation of synthesis when placed under conditions in which synthesis of primer extension product which is complementary to a target nucleic acid strand (template) is induced, i.e., in the presence of nucleotides and an agent for poly-merization, such as DNA polymerase, and at a suitable temperature and pH. The primer must be long enough to prime the synthesis of extension products in the presence of the agent for polymerization. An appropriate length of the primer is de-termined by multiple factors, including temperature, the field of application, and the source of primer.

The primer may include a forward primer (also referred to as an upstream primer or an upstream oligonucleotide), a reverse primer (also referred to as a downstream primer or a downstream oligonucleotide), or both. The amplification oligonucleotide may be an oligonucleotide having a structure known in the art, or may be synthesized by a method known in the art.

That the amplification oligonucleotide and the labeled oligonucleotide are the same means that a single oligonucleotide simultaneously acts as an amplification oligonu-cleotide that amplifies a target nucleic acid, and as a labeled oligonucleotide that generates a signal in the presence of the target nucleic acid. As one example, the labeled oligonucleotide may be hybridized to the target nucleic acid and extended, thereby generating a signal.

Note that the composition for detecting a target nucleic acid used in the present disclosure does not necessarily provide a signal at any temperature in the presence of the target nucleic acid.

In one embodiment, even when the signal generation mechanisms of the compositions for detecting target nucleic acids are the same, the compositions for detecting target nucleic acids including oligonucleotides of different sequences may be considered to be different from each other. The different compositions for detecting nucleic acids have different detection temperatures from each other.

In one embodiment, the detection temperatures according to the present disclosure may be pre-determined in light of the signal-changing temperature range of each of the n compositions for detecting target nucleic acids.

In one embodiment, the signal-changing temperature range of any one of the n compositions for detecting target nucleic acids may be determined on the basis of the length and/or sequence of the duplex. In other words, by adjusting the Tm value of the duplex, the signal-changing temperature range may be predetermined.

In one embodiment, when the signal change is generated by a labeled oligonucleotide (e.g., a molecular beacon) that specifically hybridizes to the target nucleic acid, the detection of signals may be successfully achieved at a predetermined detection temperature by adjusting the Tm value of the labeled oligonucleotide.

In one embodiment, when the signal is provided by a duplex generated in the presence of the target nucleic acid, the detection of signals is successfully achieved at a predetermined temperature by adjusting the Tm value of the duplex.

As described above, the detection temperature is determined in view of the signal-changing temperature range that varies depending on the duplex provided by the composition for detecting a target nucleic acid.

In one embodiment, the detection temperature of any one of the n compositions for the detection of n target nucleic acids may be predetermined within a signal-changing temperature range that does not overlap with the signal-changing temperature ranges of the other compositions

In one embodiment, the detection temperatures assigned to the compositions for detecting the target nucleic acids are different from each other by at least 2° C., at least 3° C., at least 4° C., at least 5° C., at least 7° C., at least 8° C., at least 9° C., at least 10° C., at least 11° C., at least 12° C., at least 15° C., at least 20° C., or more.

In one embodiment, the n detection temperatures may be selected from within a temperature range of 45° C. to 97° C., 45° C. to 96° C., 45° C. to 95° C., 45° C. to 94° C., 45° C. to 93° C., 45° C. to 92° C., 45° C. to 91° C., 45° C. to 90° C., 46° C. to 97° C., 46° C. to 96° C., 46° C. to 95° C., 46° C. to 94° C., 46° C. to 93° C., 46° C. to 92° C., 46° C. to 91° C., 46° C. to 90° C., 47° C. to 97° C., 47° C. to 96° C., 47° C. to 95° C., 47° C. to 94° C., 47° C. to 93° C., 47° C. to 92° C., 47° C. to 91° C., 47° C. to 90° C., 48° C. to 97° C., 48° C. to 96° C., 48° C. to 95° C., 48° C. to 94° C., 48° C. to 93° C., 48° C. to 92° C., 48° C. to 91° C., 48° C. to 90° C., 49° C. to 97° C., 49° C. to 96° C., 49° C. to 95° C., 49° C. to 94° C., 49° C. to 93° C., 49° C. to 92° C., 49° C. to 91° C., 49° C. to 90° C., 50° C. to 97° C., 50° C. to 96° C., 50° C. to 95° C., 50° C. to 94° C., 50° C. to 93° C., 50° C. to 92° C., 50° C. to 91° C., or 50° C. to 90° C.

For example, the highest detection temperature (i.e., the n^{th} detection temperature) among the n detection temperatures may be selected from within a temperature range of 70° C. to 97° C., 70° C. to 95° C., 70° C. to 93° C., 70° C. to 90° C., 73° C. to 97° C., 73° C. to 95° C., 73° C. to 93° C., 73° C. to 90° C., 75° C. to 97° C., 75° C. to 95° C., 75° C. to 93° C., 75° C. to 90° C., 78° C. to 97° C., 78° C. to 95° C., 78° C. to 93° C., 78° C. to 90° C., 80° C. to 97° C., 80° C. to 95° C., 80° C. to 93° C., 80° C. to 90° C., 83º° C. to 97° C., 83° C. to 95° C., 83° C. to 93° C., 83° C. to 90° C., 85° C. to 97° C., 85° C. to 95° C., 85° C. to 93° C., or 85° C. to 90° C.

For example, the lowest detection temperature (i.e., a first detection temperature) among the n detection temperatures may be selected from within a temperature range of 45° C. to 70° C., 45° C. to 68° C., 45° C. to 65° C., 45° C. to 63° C., 45° C. to 60° C., 45° C. to 58° C., 45° C. to 55° C., 48° C. to 70° C., 48° C. to 68° C., 48° C. to 65° C., 48° C. to 63° C., 48° C. to 60° C., 48° C. to 58° C., 48° C. to 55° C., 50° C. to 70° C., 50° C. to 68° C., 50° C. to 65° C., 50° C. to 63° C., 50° C. to 60° C., 50° C. to 58° C., or 50° C. to 55° C.

For example, intermediate detection temperatures (for example, from a second detection temperature to the (n−1)^{th} detection temperature) among the n detection temperatures may be selected from within a temperature range of 55° C. to 85° C., 55° C. to 83° C., 55° C. to 80° C., 55° C. to 78° C., 55° C. to 7.5° C., 55° C. to 73° C., 55° C. to 70° C., 55° C. to 68° C., 55° C. to 65° C., 55° C. to 63° C., 55° C. to 60° C., 58° C. to 85° C., 58° C. to 83° C., 58° C. to 80° C., 58° C. to 78° C., 58° C. to 75° C., 58° C. to 73° C., 58° C. to 70° C., 58° C. to 68° C., 58° C. to 65° C., 58° C. to 63° C., 58° C. to 60° C., 60° C. to 85° C., 60° C. to 83° C., 60° C. to 80° C., 60° C. to 78° C., 60° C. to 75° C., 60° C. to 73° C., 60° C. to 70° C., 60° C. to 68° C., 60° C. to 65° C., 60° C. to 63° C., 63° C. to 85° C., 63º° C. to 83° C., 63° C. to 80° C., 63° C. to 78° C., 63° C. to 75° C., 63° C. to 73° C., 63° C. to 70° C., 63° C. to 68° C., 63° C. to 65° C., 65° C. to 85° C., 65° C. to 83° C., 65° C. to 80° C., 65° C. to 78° C., 65° C. to 75° C., 65° C. to 73° C., 65° C. to 70° C., 65° C. to 68° C., 68° C. to 85° C., 68° C. to 83° C., 68° C. to 80° C., 68° C. to 78° C., 68° C. to 75° C., 68° C. to 73° C., 68° C. to 70° C., 70° C. to 85° C., 70° C. to 83° C., 70° C. to 80° C., 70° C. to 78° C., 70° C. to 75° C., or 70° C. to 73° C.

In one embodiment, the n target nucleic acids are each assigned to n detection temperatures, n compositions for detecting the n target nucleic acids appropriate for the n detection temperatures are prepared, and then step (a) may be performed.

In one embodiment, when n is 3, the first detection temperature may be selected from within a temperature range of 50° C. to 60° C., the second detection temperature may be selected from within a temperature range of 65° C. to 75° C., and the third detection temperature may be selected from within a temperature range of 80° C. to 95° C.

In the step (a), signals are detected at the n detection temperatures during the in-cubating.

In one embodiment, the detection of signals may be carried out in each cycle or selected cycles, or an end-point cycle of reaction.

In one embodiment, the detection of signals may be carried out in at least one cycle. For example, the signals may be detected at n detection temperatures in one cycle selected or at n detection temperatures in each of two cycles selected. For example, when n is 3 and signals are detected in cycle 1 and cycle 30, the signals (i.e., a first signal, a second signal, and a third signal) are detected at a first detection temperature, a second detection temperature, and a third detection temperature in cycle 1, and the signals are detected at a first detection temperature, a second detection temperature, and a third detection temperature in cycle 30.

In one embodiment, the detection of the signals may be carried out in at least two cycles.

In one embodiment, the signal change may be measured using the signals detected in the at least two cycles. For example, the amplification of nucleic acids may be carried out over 30 cycles, 40 cycles, 45 cycles, or 50 cycles of PCR, and in each cycle, signals may be measured at n detection temperatures. Then, the values of signals detected at each detection temperature in a plurality of cycles may be depicted as an amplification curve (a collection of data points of cycles and RFUs in cycles) at each detection temperature. As a specific example, when n is 3, an amplification curve at the first detection temperature, an amplification curve at the second detection temperature, and an amplification curve at the third detection temperature may be obtained, and then change in signal may be obtained from each of the amplification curves.

The term “amplification curve” as used herein refers to a curve resulting from a signal-generation reaction, in particular an amplification reaction of a target nucleic acid. The amplification curve includes a curve resulting from a reaction in the presence of the target nucleic acid in the sample, or a curve or line resulting from a reaction in the absence of the target nucleic acid in the sample.

In one embodiment, a signal change and/or a constant signal may be measured from an indicator of amplification of a target nucleic acid.

As used herein, the term “indicator of amplification” refers to any indicator which is closely related to the occurrence of amplification of a target nucleic acid and obtainable from the signal provided in step (a). The indicator of amplification may refer to a value which is generated dependently on the amplification of a target nucleic acid. The indicator of amplification may be an indicator having larger values as the target nucleic acid is amplified (i.e., as the amount of the target nucleic acid increases), or may be an indicator having smaller values as the target nucleic acid is amplified. The indicator of amplification may be any indicator as long as it indicates amplification of the target nucleic acid.

In one embodiment, the indicator of amplification may include one obtained from an amplification curve or a melting curve. In particular, the indicator of amplification may include a signal value (e.g., RFU) in a particular cycle, a signal value in each cycle, a difference in signal values between particular cycles, or a difference between a reference signal value and a signal value in a particular cycle in an amplification curve, or height, width or area of the maximum melting peak in a melting curve. In one embodiment, examples of the indicator of amplification include, without limitation, Ct (cycle threshold) value, ARFU (e.g., difference in RFUs in two cycles, difference between a reference RFU and a RFU in a particular cycle, etc.), RFU ratio (e.g., ratio of RFUs in two cycles or ratio of RFUs between a reference RFU and a RFU in a particular cycle, etc.), and height/area/width of the maximum melting peak in a melting curve.

In one embodiment, the indicator of amplification is the Ct value or Cq value. The concept of the Ct value and Cq value are well known in the art.

In one embodiment, the indicator of amplification is the ARFU or RFU ratio between RFU values obtained in an amplification reaction. For example, the indicator of ampli-fication is the difference (subtraction) or ratio between RFUs in two cycles, or the difference (subtraction) or ratio between a RFU in a particular cycle and a reference RFU.

The method according to the present disclosure exploits the fact that the composition for detecting a target nucleic acid provides a signal change depending on the presence of the target nucleic acid, only at a corresponding detection temperature. In one embodiment, the method according to the present disclosure can measure a signal change using signal values detected at detection temperatures in at least two cycles. In another embodiment, the method according to the present disclosure can measure a signal change by using a signal value detected at a detection temperature in one cycle (i.e., a signal value detected in step (a)), and a reference signal value.

In one embodiment, when signals are detected in a plurality of cycles in step (a), the first cycle and the end cycle in which signals are detected may be selected to be separated from each other by at least 1 cycle to at least 20 cycles. In particular, the first cycle and the end cycle in which the signals are detected may be selected to be separated from each other by 1 cycle, 2 cycles, 3 cycles, 4 cycles, 5 cycles, 6 cycles, 7 cycles, 8 cycles, 9 cycles, 10 cycles, 11 cycles, 12 cycles, 13 cycles, 14 cycles, 15 cycles, 16 cycles, 17 cycles, 18 cycles, 19 cycles, 20 cycles, or more, and more specifically, by 5 cycles, 10 cycles, 15 cycles, 20 cycles, 30 cycles, or more.

In one embodiment, the detection of signals may be performed in any of intermediate cycles including an exponential phase region, or in any of late cycles including a plateau region. For example, signals may be detected in two cycles, one cycle being any of initial cycles including a baseline region and the other cycle being any of intermediate or late cycles, or one cycle being any of intermediate cycles and the other cycle being any of intermediate or late cycles.

In one embodiment, the initial cycles include cycles from cycle 1 to any cycle close to the value obtained by dividing the end cycle by 3. For example, when the end cycle is 45, 45 divided by 3 equals to 15, and thus, the initial cycles may be determined to be from cycle 1 to cycle 20, cycle 1 to cycle 15, cycle 1 to cycle 10, or cycle 1 to cycle 5. The intermediate cycles may be cycles close to a value obtained by dividing the end cycle by 2. For example, when the end cycle is cycle 45, 45 divided by 2 equals to 22.5, and thus, the intermediate cycles may be from cycle 16 to cycle 30, cycle 18 to cycle 30, cycle 20 to cycle 30, cycle 16 to cycle 27, cycle 18 to cycle 27, cycle 20 to cycle 27, cycle 16 to cycle 25, cycle 18 to cycle 25, or cycle 20 to cycle 25. The late cycles may be the end cycle of, or cycles close to the end cycle of an amplification reaction. For example, when the end cycle is cycle 45, the late cycles may be from cycle 31 to cycle 45, cycle 35 to cycle 45, cycle 38 to cycle 45, cycle 40 to cycle 45, or cycle 43 to cycle 45. The initial cycles, intermediate cycles and late cycles may vary depending on the end cycle of the amplification reaction.

In one embodiment, the signal change may be measured using a signal value detected in at least one cycle and a “reference signal value”. The reference signal value may refer to a value that can be used to confirm a signal change dependent on the presence of the target nucleic acid through a separate reaction.

In one embodiment, the reference signal value may be obtained from a reaction in the absence of a corresponding target nucleic acid at a corresponding detection temperature. For example, the reference signal value may be a “signal value at a detection temperature” in the absence of the target nucleic acid.

In one embodiment, there are n reference signal values for n detection temperatures.

In one embodiment, the “signal value at a detection temperature (e.g., an i^{th} detection temperature)” detected in the absence of the target nucleic acid (e.g., an i^{th} target nucleic acid) may be obtained through a separate negative control reaction.

In one embodiment, the reference signal value may be obtained by performing a negative control reaction at the same time as or separately from the method according to the present disclosure.

In one embodiment, the reference signal value may be obtained through a negative control reaction. According to certain embodiments, the reference signal value at the i^{th} detection temperature may be obtained by mixing a sample free of the i^{th} target nucleic acid (e.g., distilled water) with n compositions for detecting n target nucleic acids and detecting signals at the i^{th} detection temperature while amplifying target nucleic acids. Here, the detection of signals may be performed in any cycle. Specifically, a signal value detected in any of the initial cycles or late cycles of the negative control reaction may be used as the reference signal value. More specifically, a signal value detected in the same cycle as the cycle in which the signal is detected in step (a) may be used as the reference signal value.

In one embodiment, the reference signal value may be obtained through a positive control reaction. According to certain embodiments, the reference signal value may be obtained by mixing a sample containing an i^{th} target nucleic acid with the composition for detecting an i^{th} target nucleic acid (particularly, n compositions for detecting n target nucleic acids) and detecting a signal at an i^{th} detection temperature while amplifying the i^{th} target nucleic acids.

When the reference signal value is obtained through the positive control reaction, the cycle in which a signal value is detected may be in a baseline region of the reaction. The baseline region refers to a region in which a signal (e.g., a fluorescent signal) remains substantially constant over the initial cycles of an amplification reaction (e.g., PCR). In this region, since the level of amplification products is not enough to be de-tectable, most of the fluorescent signals in this region are attributed to the fluorescent signal inherent to the reaction sample, and to the background signal including the fluorescent signals of the measurement system itself. In other words, a signal value detected in a cycle in the baseline region of the positive control reaction is substantially identical to the reference signal value obtained from a reaction in the absence of the target nucleic acid (e.g., the negative control reaction).

In one embodiment, a signal change may be measured through a difference between the reference signal value and the signal value detected in step (a).

In one embodiment, the reference signal value may be a threshold value prede-termined from a negative control reaction, by taking into consideration the background signal and sensitivity of the detector, or characteristics of the labels used. Using the threshold value, significance of a signal change may be determined. The threshold value may be determined by any threshold setting method known in the art. For example, the threshold value may be determined in view of the background signal, sensitivity, label characteristics, signal variation of a detector, or margin of errors, and the like

In one embodiment, when the signal value detected in step (a) is equal to or greater than the threshold value as the reference signal value, it may be determined that the signal has changed.

In one embodiment, detection of signals at each of the n detection temperatures may be performed using a single type of detector.

In one embodiment, the single type of detector is one detector. In one embodiment, the signals from the labels in each of the labeled oligonucleotides included in the n compositions for detecting target nucleic acids are not differentiated from each other by a single type of detector with respect to each target nucleic acid.

As used herein, a single or one type of fluorescent label refers to a fluorescent label that has identical or substantially identical signal characteristics (e.g., optical characteristics, emission wavelength, and electric signals). For example, FAM and CAL Fluor 610 provide different types of signals.

As used herein, the single or one type of fluorescent label means that signals from the fluorescent label are not differentiated from each other, using a detection channel. Such a single or one type of fluorescent label is not based on the chemical structure of the fluorescent label, and even when two fluorescent labels having different chemical structures are not differentiated using a detection channel, they are considered as one type.

According to the present disclosure, signals generated from the n compositions for detecting target nucleic acids that include one type of fluorescent label in common are not differentiated by one detection channel.

The term “detection channel” as used herein refers to a means for detecting a signal from a single type of a fluorescent label. Thermocyclers usable in the art, e.g., ABI 7500 (Applied Biosystems), QuantStudio (Applied Biosystems), CFX96 (Bio-Rad Laboratories), Cobas z 480 (Roche), LightCycler (Roche), etc. include a several channels (e.g., optical diodes) for detecting signals from a few different types of fluorescent labels, and these channels correspond to the detection channel as used herein.

The detection channel as used herein includes a means for detecting signals. For example, the detection channel may be an optical diode capable of detecting a fluorescent signal at a particular wavelength.

In one embodiment, the signals detected at the n detection temperatures are not differentiated from each other by the single type of detector.

### Step (b): Determining the Presence of Target Nucleic Acid

After detection of signals, the presence of n target nucleic acids is determined from the signals detected in the step (a).

In one embodiment, the presence of the i^{th} target nucleic acid is determined by the signal change detected at the i^{th} detection temperature. For example, a signal change is measured from the signals detected at the i^{th} detection temperature, to determine the presence of the i^{th} target nucleic acid.

In one embodiment, when a change in signal at the i^{th} detection temperature is detected, it may be determined that the i^{th} target nucleic acid is present.

In one embodiment, when a change in signal at the i^{th} detection temperature is not detected, i.e., the signal is constant at the i^{th} detection temperature, it may be de-termined that the i^{th} target nucleic acid is absent.

In one embodiment, the signal change can be measured using signals detected in at least two cycles, or a signal value detected in at least one cycle and a reference signal value.

Determining the presence of the target nucleic acid from signals detected at each detection temperature may be carried out by the process described in the step (a) for measuring a signal change, e.g., a method using an indicator of amplification, or any other method known in the art.

In certain embodiments, when n is 3 and detection of signals is carried out in cycle 10, cycle 20, and cycle 30, the presence of the first target nucleic acid may be de-termined from signals detected at a first detection temperature (a first signal in cycle 10, a first signal in cycle 20, and a first signal in cycle 30), the presence of the second target nucleic acid may be determined from signals detected at a second detection temperature (a second signal in cycle 10, a second signal in cycle 20, and a second signal in cycle 30), and the presence of the third target nucleic acid may be determined from signals detected at a third detection temperature (a third signal in cycle 10, a third signal in cycle 20, and a third signal in cycle 30).

In certain embodiments, when n is 4 and detection of signals is performed in cycle 30, the presence of the first target nucleic acid is determined from a signal detected at a first detection temperature (i.e., a first signal in cycle 30) and a reference signal value, the presence of the second target nucleic acid is determined from a signal detected at a second detection temperature (i.e., a second signal in cycle 30) and a reference signal value, and the presence of the third target nucleic acid is determined from a signal detected at a third detection temperature (i.e., a third signal in cycle 30) and a reference signal value.

In one embodiment, the reference signal value may be obtained through a separate negative control reaction or positive control reaction.

In one embodiment, the method according to the present disclosure may be performed along with a negative control reaction. A signal value detected in the negative control reaction may be used as a reference signal value. For example, a signal detected at an i^{th} detection temperature in one cycle (e.g., the end cycle) of a reaction comprising the composition for detecting an i^{th} target nucleic acid, may be compared with a signal detected at the same detection temperature (i.e., the i^{th} detection temperature) in the same cycle (e.g., the end cycle) of the negative control reaction to determine whether or not the signal has changed.

In certain embodiments, when n is 3 and a signal is detected in cycle 30, the presence of the first target nucleic acid may be determined from a signal detected at the first detection temperature (i.e., a first signal in cycle 30) and a first reference signal value (e.g., a signal detected at the first detection temperature in cycle 30 of a negative control reaction), the presence of the second target nucleic acid may be determined from a signal detected at the second detection temperature (i.e., a second signal in cycle 30) and a second reference signal value (e.g., a signal detected at the second detection temperature in cycle 30 of a negative control reaction), and the presence of the third target nucleic acid may be determined from a signal detected at the third detection temperature (i.e., a third signal in cycle 30) and a third reference signal value (e.g., a signal detected at the third detection temperature in cycle 30 of a negative control reaction).

In one embodiment, the method according to the present disclosure may be performed along with a positive control reaction. A signal value detected in the positive control reaction may be used as a reference signal value. For example, a first signal detected at an i^{th} detection temperature in one cycle, e.g., cycle 30, may be compared with a signal detected at the i^{th} detection temperature in a cycle, e.g., cycle 1 prior to cycle 30 of a positive control reaction to determine whether the signal has changed.

In one embodiment, when signal detection is performed in one cycle in step (a) and a signal change is measured using a reference signal value obtained through a positive control reaction, the signal detection in the positive control reaction to obtain the reference signal value may be performed in a cycle that is at least 30 cycles, at least 20 cycles, at least 10 cycles, or at least 5 cycles prior to the cycle in which the signal detection is performed in step (a).

In certain embodiments, when n is 3 and signal detection is performed in cycle 30, the presence of the first target nucleic acid may be determined from a signal detected at the first detection temperature (i.e., a first signal in cycle 30) and a first reference signal value (e.g., a signal detected at the first detection temperature in cycle 1 of a positive control reaction for the first target nucleic acid), the presence of the second target nucleic acid may be determined from a signal detected at the second detection temperature (i.e., a second signal in cycle 30) and a second reference signal value (e.g., a signal detected at the second detection temperature in cycle 1 of a positive control reaction for the second target nucleic acid), and the presence of the third target nucleic acid may be determined from a signal detected at the third detection temperature (i.e., a third signal in cycle 30) and a third reference signal value (e.g., a signal detected at the third detection temperature in cycle 1 of a positive control reaction for the third target nucleic acid).

IV. Method for Detecting Target Nucleic Acid using LPHO

In a fourth aspect of the present disclosure, there is provided a method for detecting a target nucleic acid in a sample using a Labeled Portion Hybridizing Oligonucleotide (LPHO),
(a) providing a fragment produced by an enzymatic cleavage reaction of an oligonu-cleotide depending on the presence of the target nucleic acid in the sample;
(b) hybridizing the fragment with a Capturing and Templating Oligonucleotide (CTO);
wherein the CTO comprises in a 3′ to 5′ direction: (i) a capturing portion comprising a hybridizing nucleotide sequence with the fragment, and (ii) a templating portion comprising a non-hybridizing nucleotide sequence with the fragment,
wherein the CTO has a reporter molecule and a quencher molecule defining a labeled portion,
wherein the fragment is hybridized with the capturing portion of the CTO;
(c) performing an extension reaction using the resultant of the step (b) and the DNA polymerase having 5′ nuclease activity in the presence of a Labeled Portion Hybridizing Oligonucleotide (LPHO);
wherein the LPHO comprises a hybridizing nucleotide sequence with the labeled portion of the CTO,
wherein when the target nucleic acid is present in the sample, the fragment hybridized with the capturing portion of the CTO is extended to generate an extended strand complementary to the CTO, thereby generating an extended duplex between the extended strand and the CTO, wherein the generation of the extended duplex prevents the formation of a CTO/LPHO hybrid between the labeled portion of the CTO and the LPHO,
wherein when the target nucleic acid is not present in the sample, the extended strand is not generated, and instead the CTO/LPHO hybrid is formed,
wherein the extended duplex has a melting temperature (Tm) that is different from a Tm of the CTO/LPHO hybrid; and
(d) detecting the presence of the extended duplex;
wherein the extended duplex is detected by measuring a signal provided from the extended duplex,
wherein the measurement is performed at a temperature at which the signal intensity from the extended duplex is different from a signal intensity from the CTO/LPHO hybrid, and
wherein the presence of the extended duplex indicates the presence of the target nucleic acid.

Since the fourth aspect of the present disclosure relates to a method for detecting extended duplexes using LPHO described in the first and second aspects of the present disclosure, the common descriptions between them are omitted in order to avoid undue redundancy leading to the complexity of this specification.

First, in step (a), there is provided a fragment produced by enzymatic cleavage of an oligonucleotide depending on the presence of the target nucleic acid in the sample.

The fragment may include any fragment produced by enzymatic cleavage of an oligonucleotide depending on the presence of the target nucleic acid, such as a PTO fragment as described above.

The provision of the fragment in the step (a) can be achieved by any of various oligonucleotide cleavage reactions known in the art.

For example, as disclosed in U.S. Pat. No. 7,482,121, two oligonucleotides are hybridized with the same strand of the target nucleic acid in a non-overlapping manner, to form a cleavage structure. The cleavage structure is then cleaved using an enzyme, such as the thermostable FEN-1 nuclease, to provide a fragment. The cleavage structure includes a double-stranded hybrid comprising a pseudo-Y structure, a gap, or a nick. As another example, the fragment may be provided by a cleavage reaction of the mediation oligonucleotide as described above. For example, Invader assay (U.S. Pat. No. 5,691,142) provides a fragment by cleavage of the mediating oligonucleotide. As another example, a primer comprising a restriction enzyme recognition sequence is hybridized with a target nucleic acid to generate an amplification product including the restriction enzyme recognition sequence, and the restriction enzyme recognition sequence in the amplification product is then cleaved to provide a fragment.

As described above, endonuclease or exonuclease may be used to cleave the oligonu-cleotide. Examples of endonucleases include restriction enzymes, RNA endonucleases, and DNA endonucleases; and examples of exonucleases include, but not limited to, 5′-exonuclease and 3′-exonuclease.

In one embodiment, the oligonucleotide cleaved by the enzymatic cleavage reaction may be the target nucleic acid itself.

The steps (b) to (d) of the fourth aspect of the present disclosure can be described in detail with reference to the steps (c) to (e) of the first aspect of the present disclosure, respectively.

Hereinafter, the present invention will be described in more detail through embodiments. The following embodiments are provided to describe the present invention in further details, and it will become apparent to one of ordinary skill in the art in the technical field to which this invention belongs that the scope of the present invention as suggested in the appended claims is not limited by the following embodiments.

### Mode for the Invention

### EXAMPLES

### Example 1: Detection of Single Target Nucleic Acid

In this Example, it was investigated whether the L-PTOCE assay can be used to detect a target nucleic acid in real-time.

For this, two types of CTOs having the same sequence with the reporter molecule and the quencher molecule labeled at different positions, and four types of LPHOs of different lengths for the two types of CTOs were prepared. Subsequently, four combinations of the two types of CTOs and four types of LPHOs were tested for detection of the target nucleic acid.

### 1-1. Preparation of Target Nucleic Acid and Oligonucleotides

As a target nucleic acid, the genomic DNA of Mycoplasma genitalium (MG) (Accession number: ATCC 33530) were used. For detection of MG target nucleic acids, a forward primer (i.e., upstream primer), a reverse primer (i.e., downstream primer), and a PTO were prepared as shown in Table 3.

TABLE 3

| SEQ | | | |
|---|---|---|---|
| ID | Oligo | Tm | |
| No. | Type | (° C.) | Sequence (5′-3′) |
| 1 | MG- | 62 | TGATATCCATCCTAAGACTAATCGTIIIIIAGTTGAAAC |
| | Forward | | |
| | primer | | |
| 2 | MG- | 62 | CCAATTACCTTTCCTCCATCGIIIIIGCTGAGAAA |
| | Reverse | | |
| | primer | | |
| 3 | MG-PTO | 74 | CTTCGATCGCGTCACGGTGTTGGTGCATCAGTTGTTA |
| | | | ATG [Spacer C3] |

The underlined character indicates the 5′-tagging portion of the PTO.

Subsequently, two types of CTOs and four types of LPHOs were prepared as shown in Table 4.

TABLE 4

| SEQ | | | |
|---|---|---|---|
| ID | Oligo | Tm | |
| NO. | Type | (° C.) | Sequence (5′-3′) |
| 4 | CTO-1 | 78 | [BHQ-1]GGCTCGCATAGATTCATGGC[T(CAL |
| | | | Fluor Orange |
| | | | 560)]GGGTGACGCGATCGAAG[Spacer C3] |
| 5 | LPHO-1A | 72 | GTCACCCAGCCATGAATCTATGCGAGCC |
| | | | [Spacer C3] |
| 6 | LPHO-1B | 72 | CTTIGATCGCGTIACCCAGCCATGAATCTATGC |
| | | | GAGCC [Spacer C3] |
| 7 | CTO-2 | 78 | GGCTCGCATAGATTCATGGC[T(CAL Fluor |
| | | | Orange 560)]GGGTGACGCGATCGAAG[BHQ-1] |
| 8 | LPHO-2A | 72 | CTTCGATCGCGTCACCCAGCCATGAATC |
| | | | [Spacer C3] |
| 9 | LPHO-2B | 72 | CTTCGATCGCGTCACCCAGCCATGAITCTATGI |
| | | | GAGCC [Spacer C3] |

As shown in Table 4, the first CTO (hereinafter referred to as CTO-1) has a quencher molecule (BHQ-1) linked to the 5′-end of the targeting portion and a reporter molecule (CAL Fluor Orange 560) linked to the 3′-end of the targeting portion; the second CTO (hereinafter referred to as CTO-2) has a quencher molecule (BHQ-1) linked to the 3′-end of the capturing portion, and a reporter molecule (CAL Fluor Orange 560) linked within the templating portion.

Further, two types of LPHOs (hereinafter referred to as LPHO-1A and LPHO-1B) of different lengths comprising a hybridizing nucleotide sequence with the labeled portion of CTO-1, and two types of LPHOs (hereinafter referred to as LPHO-2A and LPHO-2B) of different lengths comprising a hybridizing nucleotide sequence with the labeled portion of CTO-2 were prepared. The LPHO-1A and LPHO-2A are shorter than the LPHO-1B and LPHO-2B in lengths, respectively.

The 3′-ends of the PTO, CTO, and LPHO were each blocked by Spacer C3 to prohibit its extension by DNA polymerase.

### 1-2. Real-Time PCR

Real-time PCR was conducted for four combinations of one of the two types of CTOs and one of the four types of LPHOs as follows.
Combination 1: CTO-1 and LPHO-1A
Combination 2: CTO-1 and LPHO-1B
Combination 3: CTO-2 and LPHO-2A
Combination 4: CTO-2 and LPHO-2B

### <Combination 1>

The target nucleic acid (Tube 1:1 μg of MG genomic DNA) and distilled water (Tube 2, Negative control) were each mixed with: 5 pmole of MG-forward primer (SEQ ID NO: 1), 5 pmole of MG-reverse primer (SEQ ID NO: 2), 3 pmole of MG-PTO (SEQ ID NO: 3), 1 pmole of CTO-1 (SEQ ID NO: 4) and 3 pmole LPHO-1A (SEQ ID NO: 5), and then combined with 5 μL of 4X Enzyme Mix (20 U of Taq DNA polymerase) (Nanohelix, Korea) and 5 μL of 4X Buffer mix (final, 0.8 mM of dNTPs, 50 mM of KCl, 3.5 mM of MgCl₂) (Nanohelix, Korea), to prepare a reaction mixture in the final volume of 20 μL.

The tube 1 and tube 2 containing the reaction mixture were each placed in a real-time thermal cycler (CFX96 Real-time Cycler, Bio-Rad), and subjected to real-time PCR consisting of denaturation at 95° C. for 15 minutes, and then 50 cycles of 10 seconds at 95° C., 15 seconds at 60° C., 10 seconds at 72° C., and 5 seconds at 75° C.

Detection of signals was performed at three temperatures in each cycle as follows:
(i) 60° C. at which both the extended duplex and the CTO/LPHO hybrid remain its double-stranded state (i.e., the temperature within the first signal-constant temperature range);
(ii) 75° C. at which the extended duplex remains its double-stranded state and the CTO/LPHO hybrid dissociates into a single-stranded state (i.e., the temperature within the signal-changing temperature range); and
(iii) 95° C. at which both the extended duplex and the CTO/LPHO hybrid dissociate into a single-stranded state (i.e., the temperature within the second signal-constant temperature range).

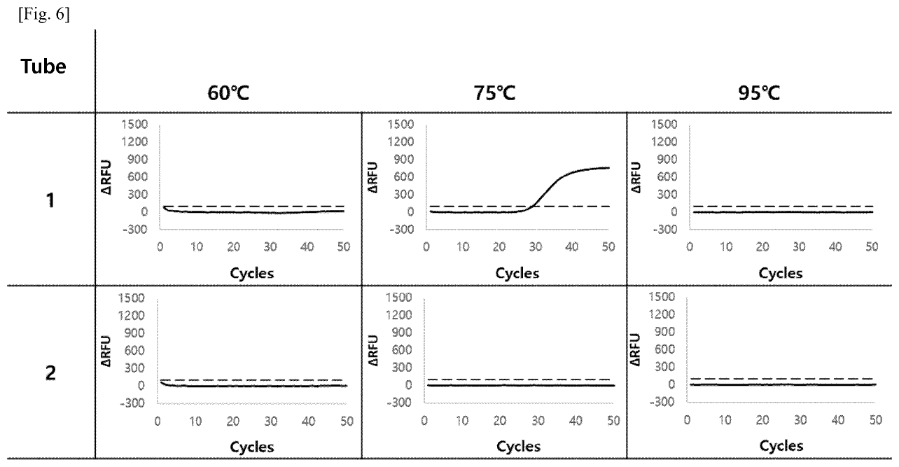

The real-time PCR results were shown in FIG. 6.

For the amplification curve at 60° C. within the first signal-constant temperature range and the amplification curve at 95° C. within the second signal-constant temperature range, the signal remained constant without any changes even though the target nucleic acid was amplified. On the other hand, for the amplification curve at 75° C. within the signal-changing temperature range, the signal changed as the target nucleic acid was amplified.

Meanwhile, the negative control provided a constant signal at 60° C., 75° C. and 95° C. That is, no signal change was detected.

As described above, it was found that the L-PTOCE assay according to the present disclosure can measure a signal (i.e., a change in the signal) indicating the presence of the target nucleic acid using a reference signal value obtained from a negative control reaction. The signal was considered to have changed if the signal values at 60° C., 75° C., and 95° C. were higher than RFU 100, a threshold based on the signal value of the negative control reaction (i.e., RFU: 0). As a result, as shown in Table 5, a change in signal was identified from a Ct value of 28.58 only at 75° C., and no change in signal was identified at 60° C. and 95° C.

TABLE 5

| | Ct (Cycle threshold) | | |
|---|---|---|---|
| Tube | 60° C. | 75° C. | 95° C. |
| 1 | N/A | 28.58 | N/A |
| 2 | N/A | N/A | N/A |

Tube 1: 1 pg of MG genomic DNA;
Tube 2: Negative control;
N/A: Not Applicable

### <Combination 2>

The target nucleic acid (Tube 1:50 μg of MG genomic DNA) and distilled water (Tube 2, Negative control) were each mixed with: 5 pmole of MG-forward primer (SEQ ID NO: 1), 5 pmole of MG-reverse primer (SEQ ID NO: 2), 3 pmole of MG-PTO (SEQ ID NO: 3), 1 pmole of CTO-1 (SEQ ID NO: 4) and 3 pmole LPHO-1B (SEQ ID NO: 6), and then combined with 5 μL of 4X Enzyme Mix (20 U of Taq DNA polymerase) (Nanohelix, Korea) and 5 μL of 4X Buffer mix (final, 0.8 mM of dNTPs, 50 mM of KCl, 3.5 mM of MgCl₂) (Nanohelix, Korea), to prepare a reaction mixture in the final volume of 20 μL.

The tube 1 and tube 2 containing the reaction mixture were each placed in a real-time thermal cycler (CFX96 Real-time Cycler, Bio-Rad), and subjected to real-time PCR consisting of denaturation at 95° C. for 15 minutes, and then 50 cycles of 10 seconds at 95° C., 15 seconds at 60° C., 10 seconds at 72° C., and 5 seconds at 75° C.

Detection of signals was performed at three temperatures in each cycle as follows:
(i) 60° C. at which both the extended duplex and the CTO/LPHO hybrid remain its double-stranded state (i.e., the temperature within the first signal-constant temperature range);
(ii) 75° C. at which the extended duplex remains its double-stranded state and the CTO/LPHO hybrid dissociates into a single-stranded state (i.e., the temperature within the signal-changing temperature range); and
(iii) 95° C. at which both the extended duplex and the CTO/LPHO hybrid dissociate into a single-stranded state (i.e., the temperature within the second signal-constant temperature range).

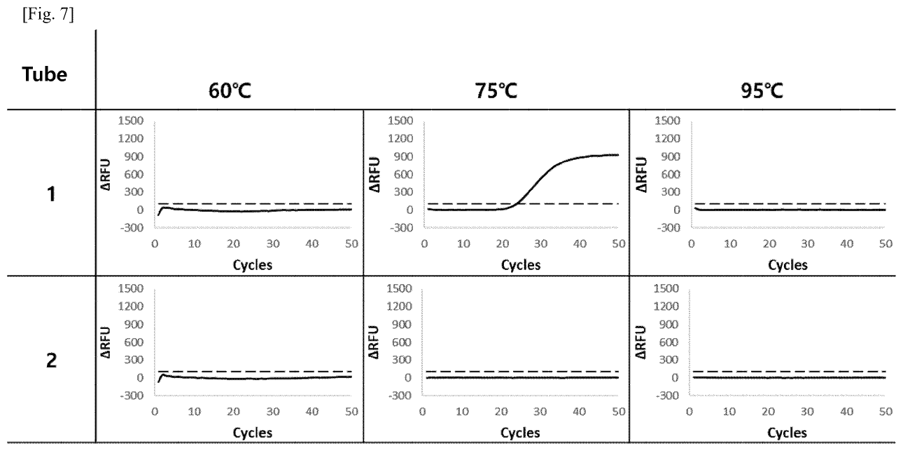

The real-time PCR results were shown in FIG. 7.

For the amplification curve at 60° C. within the first signal-constant temperature range and the amplification curve at 95° C. within the second signal-constant temperature range, the signal remained constant without any changes even though the target nucleic acid was amplified. On the other hand, for the amplification curve at 75° C. within the signal-changing temperature range, the signal changed as the target nucleic acid was amplified.

Meanwhile, the negative control provided a constant signal at 60° C., 75° C. and 95° C. That is, no signal change was detected.

As described above, it was found that the L-PTOCE assay according to the present disclosure can measure a signal (i.e., a change in the signal) indicating the presence of the target nucleic acid using a reference signal value obtained from a negative control reaction. The signal was considered to have changed if the signal values at 60° C., 75° C., and 95° C. were higher than RFU 100, a threshold based on the signal value of the negative control reaction (i.e., RFU: 0). As a result, as shown in Table 6, a change in signal was identified from a Ct value of 28.58 only at 75° C., and no change in signal was identified at 60° C. and 95° C.

TABLE 6

| | Ct (Cycle threshold) | | |
|---|---|---|---|
| Tube | 60° C. | 75° C. | 95° C. |
| 1 | N/A | 23.33 | N/A |
| 2 | N/A | N/A | N/A |

Tube 1: 50 pg of MG genomic DNA;
Tube 2: Negative control;
N/A: Not Applicable

### <Combination 3>

The target nucleic acid (Tube 1:5 μg of MG genomic DNA) and distilled water (Tube 2, Negative control) were each mixed with: 5 pmole of MG-forward primer (SEQ ID NO: 1), 5 pmole of MG-reverse primer (SEQ ID NO: 2), 3 pmole of MG-PTO (SEQ ID NO: 3), 1 pmole of CTO-2 (SEQ ID NO: 7) and 3 pmole LPHO-2A (SEQ ID NO: 8), and then combined with 5 μL of 4X Enzyme Mix (20 U of Taq DNA polymerase) (Nanohelix, Korea) and 5 μL of 4X Buffer mix (final, 0.8 mM of dNTPs, 50 mM of KCl, 3.5 mM of MgCl₂) (Nanohelix, Korea), to prepare a reaction mixture in the final volume of 20 μL.

The tube 1 and tube 2 containing the reaction mixture were each placed in a real-time thermal cycler (CFX96 Real-time Cycler, Bio-Rad), and subjected to real-time PCR consisting of denaturation at 95° C. for 15 minutes, and then 50 cycles of 10 seconds at 95° C., 15 seconds at 60° C., 10 seconds at 72° C., and 5 seconds at 75° C.

Detection of signals was performed at three temperatures in each cycle as follows:
(i) 60° C. at which both the extended duplex and the CTO/LPHO hybrid remain its double-stranded state (i.e., the temperature within the first signal-constant temperature range);
(ii) 75° C. at which the extended duplex remains its double-stranded state and the CTO/LPHO hybrid dissociates into a single-stranded state (i.e., the temperature within the signal-changing temperature range); and
(iii) 95° C. at which both the extended duplex and the CTO/LPHO hybrid dissociate into a single-stranded state (i.e., the temperature within the second signal-constant temperature range).

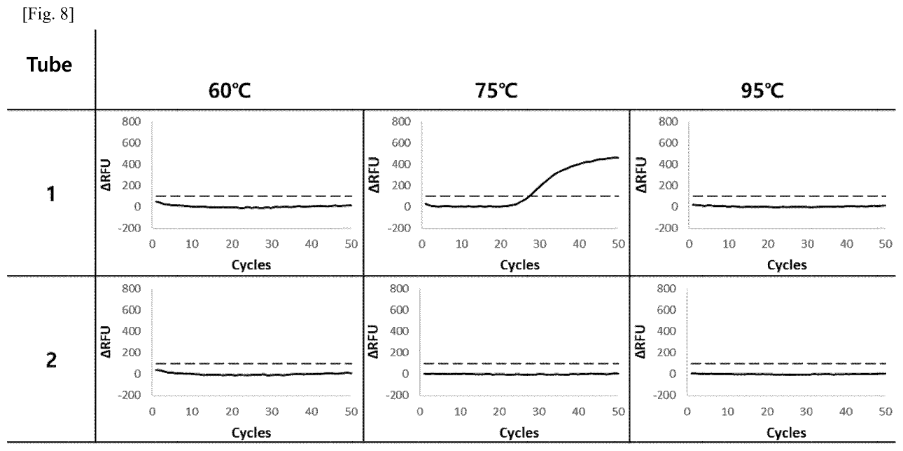

The real-time PCR results were shown in FIG. 8.

For the amplification curve at 60° C. within the first signal-constant temperature range and the amplification curve at 95° C. within the second signal-constant temperature range, the signal remained constant without any changes even though the target nucleic acid was amplified. On the other hand, for the amplification curve at 75° C. within the signal-changing temperature range, the signal changed as the target nucleic acid was amplified.

Meanwhile, the negative control provided a constant signal at 60° C., 75° C. and 95° C. That is, no signal change was detected.

As described above, it was found that the L-PTOCE assay according to the present disclosure can measure a signal (i.e., a change in the signal) indicating the presence of the target nucleic acid using a reference signal value obtained from a negative control reaction. The signal was considered to have changed if the signal values at 60° C., 75° C., and 95° C. were higher than RFU 100, a threshold based on the signal value of the negative control reaction (i.e., RFU: 0). As a result, as shown in Table 7, a change in signal was identified from a Ct value of 28.58 only at 75° C., and no change in signal was measured at 60° C. and 95° C.

TABLE 7

| | Ct (Cycle threshold) | | |
|---|---|---|---|
| Tube | 60° C. | 75° C. | 95° C. |
| 1 | N/A | 27.25 | N/A |
| 2 | N/A | N/A | N/A |

Tube 1: 5 pg of MG genomic DNA;
Tube 2: Negative control;
N/A: Not Applicable

### <Combination 4>

The target nucleic acid (Tube 1:5 μg of MG genomic DNA) and distilled water (Tube 2, Negative control) were each mixed with: 5 pmole of MG-forward primer (SEQ ID NO: 1), 5 pmole of MG-reverse primer (SEQ ID NO: 2), 3 pmole of MG-PTO (SEQ ID NO: 3), 1 pmole of CTO-2 (SEQ ID NO: 7) and 3 pmole LPHO-2B (SEQ ID NO: 9), and then combined with 5 μL of 4X Enzyme Mix (20 U of Taq DNA polymerase) (Nanohelix, Korea) and 5 μL of 4X Buffer mix (final, 0.8 mM of dNTPs, 50 mM of KCl, 3.5 mM of MgCl₂) (Nanohelix, Korea), to prepare a reaction mixture in the final volume of 20 μL.

The tube 1 and tube 2 containing the reaction mixture were each placed in a real-time thermal cycler (CFX96 Real-time Cycler, Bio-Rad), and subjected to real-time PCR consisting of denaturation at 95° C. for 15 minutes, and then 50 cycles of 10 seconds at 95° C., 15 seconds at 60° C., 10 seconds at 72° C., and 5 seconds at 75° C.

Detection of signals was performed at three temperatures in each cycle as follows:
(i) 60° C. at which both the extended duplex and the CTO/LPHO hybrid remain its double-stranded state (i.e., the temperature within the first signal-constant temperature range);
(ii) 75° C. at which the extended duplex remains its double-stranded state and the CTO/LPHO hybrid dissociates into a single-stranded state (i.e., the temperature within the signal-changing temperature range); and
(iii) 95° C. at which both the extended duplex and the CTO/LPHO hybrid dissociate into a single-stranded state (i.e., the temperature within the second signal-constant temperature range).

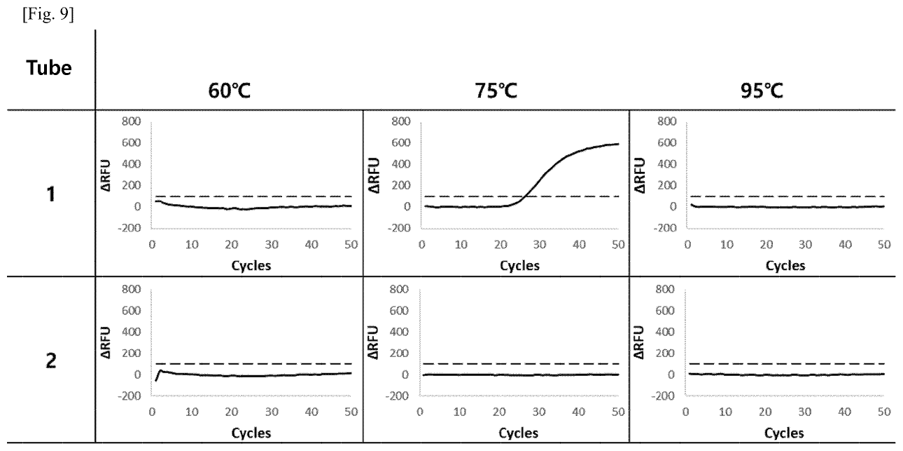

The real-time PCR results were shown in FIG. 9.

For the amplification curve at 60° C. within the first signal-constant temperature range and the amplification curve at 95° C. within the second signal-constant temperature range, the signal remained constant without any changes even though the target nucleic acid was amplified. On the other hand, for the amplification curve at 75° C. within the signal-changing temperature range, the signal changed as the target nucleic acid was amplified.

Meanwhile, the negative control provided a constant signal at 60° C., 75° C. and 95° C. That is, no signal change was detected.

As described above, it was found that the L-PTOCE assay according to the present disclosure can measure a signal (i.e., a change in the signal) indicating the presence of the target nucleic acid using a reference signal value obtained from a negative control reaction. The signal was considered to have changed if the signal values at 60° C., 75° C., and 95° C. were higher than RFU 100, a threshold based on the signal value of the negative control reaction (i.e., RFU: 0). As a result, as shown in Table 8, a change in signal was identified from a Ct value of 26.28 only at 75° C., and no change in signal was measured at 60° C. and 95° C.

TABLE 8

| | Ct (Cycle threshold) | | |
|---|---|---|---|
| Tube | 60° C. | 75° C. | 95° C. |
| 1 | N/A | 26.28 | N/A |
| 2 | N/A | N/A | N/A |

Tube 1: 5 pg of MG genomic DNA;
Tube 2: Negative control;
N/A: Not Applicable

Through the results of Combination 1 to Combination 4, it has been confirmed that the L-PTOCE assay and composition according to the present disclosure can be utilized for the detection of the target nucleic acid. Furthermore, the L-PTOCE assay and composition according to the present disclosure have been verified to be applicable as an InterSC-type signal generation method and composition.

### Example 2: Detection of Single Nucleotide Polymorphism

In this Example, it was investigated whether the L-PTOCE assay can be used to detect a single nucleotide polymorphism of the target nucleic acid.

### 2-1. Preparation of Target Nucleic Acid and Oligonucleotides

As a target nucleic acid, a synthetic RNA of the SARS-COV-2 N501Y variant, which incorporates the nucleotide sequence encoding the N501Y amino acid variation, was used. The nucleotide sequence encoding the amino acid variation N501Y has a single nucleotide polymorphism—a substitution of the nucleotide T for A at position 1,501 on the reference sequence of the S gene of SARS-COV-2 (RefSeq: NC_045512.2).

The target nucleic acid was prepared as follows:
pBluescriptIISK+plasmid having 447 bp DNA sequence (SEQ ID NO: 10) with the single nucleotide polymorphism as shown in Table 9 inserted was purchased from Bionics. Afterwards, the plasmid was used to prepare SARS-COV-2 N501Y variant synthetic RNA using the MEGAscript™ T7 Transcription Kit (Thermofisher, AM1334).

TABLE 9

| Target | SEQ ID | |
|---|---|---|
| nucleic acid | NO. | Sequence (5′-3′) |
| SARS-CoV-2 | 10 | CTGCGTTATAGCTTGGAATTCTAACAATCTTGATTCT |
| N501Y | | AAGGTTGGTGGTAATTATAATTACCTGTATAGATTGT |
| variant | | TTAGGAAGTCTAATCTCAAACCTTTTGAGAGAGATAT |
| | | TTCAACTGAAATCTATCAGGCCGGTAGCACACCTTGT |
| | | AATGGTGTTAAAGGTTTTAATTGTTACTTTCCTTTACA |
| | | ATCATATGGTTTCCAACCCACTTATGGTGTTGGTTAC |
| | | CAACCATACAGAGTAGTAGTACTTTCTTTTGAACTTC |
| | | TACATGCACCAGCAACTGTTTGTGGACCTAAAAAGTC |
| | | TACTAATTTGGTTAAAAACAAATGTGTCAATTTCAAC |
| | | TTCAATGGTTTAACAGGCACAGGTGTTCTTACTGAGT |
| | | CTAACAAAAAGTTTCTGCCTTTCCAACAATTTGGCAG |
| | | AGACATTGCTGACACTACTGATGCTGTCCGTGATCCA |
| | | CA |

The bolded character indicates the single nucleotide polymorphism.

For detecting SARS-COV-2 N501Y variant, a forward primer, a reverse primer, a PTO, a CTO and a LPHO were prepared as shown in Table 10.

TABLE 10

| Target | | | | |
|---|---|---|---|---|
| nucleic | SEQ ID | Oligo | Tm | |
| acid | No. | Type | (° C.) | Sequence (5′-3′) |
| SARS- | 11 | Forward | 64 | AAACCTTTTGAGAGAGATATTTCAAC |
| CoV-2 | | primer | | |
| N501Y | 12 | Reverse | 61.5 | ACATTTGTTTTTAACCAAATTAGTAG |
| variant | | primer | | |
| | 13 | PTO | 72.5 | CCTCAGGTGGCAATATGGTGTTGGTTACCA |
| | | | | ACCATACAGAGTAGTA [Spacer C3] |
| | 14 | CTO | 77.5 | [BHQ-1]CCTTTCGCCCACATCGT[T(CAL |
| | | | | Fluor Red 610)]GCTGCCATTGCCACCTGAGG |
| | | | | [SpacerC3] |
| | 15 | LPHO | 69 | AATGGCAGCAACGATGTGGGCGAAAGG |
| | | | | [Spacer C3] |

The underlined character indicates the 5′-tagging portion of the PTO; The bolded character indicates the single nucleotide polymorphism.

### 2-2. Real-Time Revers Transcription-PCR

Real-time RT-PCR was performed using the above oligonucleotides.

The target nucleic acid (Tube 1: 5X10³ copies of synthetic RNA of SARS-COV-2 N501Y variant), non-target nucleic acid (Tube 2:103 copies of SARS-COV-2 wild type genomic DNA (ATCC, VR-1991D)), and distilled water (Tube 3, Negative control) were each mixed with: 5 pmole of forward primer (SEQ ID NO: 11), 5 pmole of reverse primer (SEQ ID NO: 12), 3 pmole of PTO (SEQ ID NO: 13), 1 pmole of CTO (SEQ ID NO: 14), and 3 pmole LPHO (SEQ ID NO: 15), and then combined with 5 μL of 4X Enzyme Mix (20 U of Taq DNA polymerase, 30U of M-MLV reverse transcriptase) (Nanohelix, Korea) and 5 μL of 4X Buffer mix (final, 0.8 mM of dNTPs, 200 mM of KCl, 14 mM of MgCl₂) (Nanohelix, Korea), to prepare a reaction mixture in the final volume of 20 μL.

The tube 1 to tube 3 containing the reaction mixture were each placed in a real-time thermal cycler (CFX96 Real-time Cycler, Bio-Rad) and subjected to real-time RT-PCR consisting of reverse transcription at 50° C. for 20 minutes, denaturation at 95° C. for 15 minutes, and then 50 cycles of 10 seconds at 95° C., 15 seconds at 60° C., 5 seconds at 70° C., and 10 seconds at 72° C.

Detection of the signals was performed at three temperatures in each cycle as follows:
(i) 60° C. at which both the extended duplex and the CTO/LPHO hybrid remain its double-stranded state (i.e., the temperature within the first signal-constant temperature range);
(ii) 70° C. at which the extended duplex remains its double-stranded state and the CTO/LPHO hybrid dissociates into a single-stranded state (i.e., the temperature within the signal-changing temperature range); and
(iii) 95° C. at which both the extended duplex and the CTO/LPHO hybrid dissociate into a single-stranded state (i.e., the temperature within the second signal-constant temperature range).

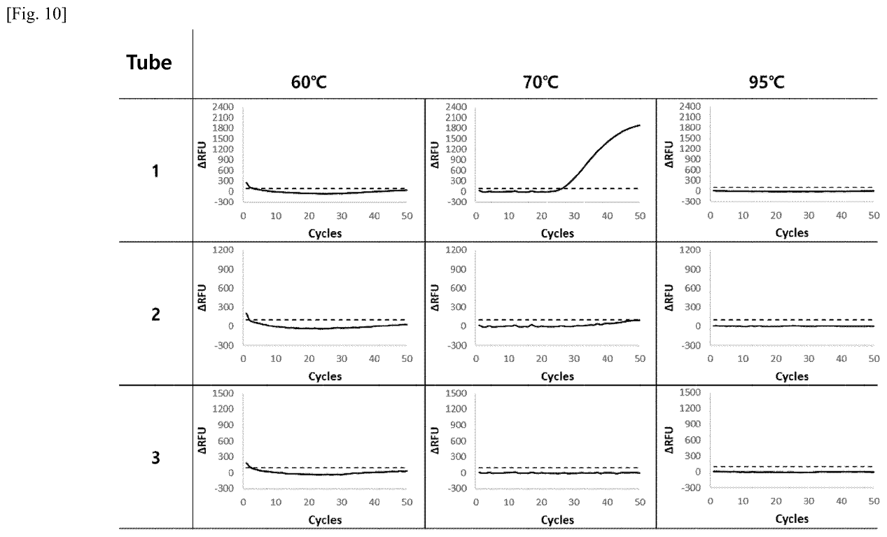

The real-time RT-PCR results were shown in FIG. 10.

The signal was considered to have changed if the signal values at 60° C., 75° C., and 95° C. were higher than RFU 100, a threshold based on the signal value of the negative control reaction (i.e., RFU: 0). As a result, as shown in FIG. 10 and Table 8, a change in signal in Tube 1 comprising the synthetic RNA of SARS-COV-2 N501Y variant was identified from a Ct value of 25.97 only at 75° C., and no change in signal was identified at 60° C. and 95° C.

Meanwhile, for tube 2 containing SARS-COV-2 wild type genomic RNA and tube 3 as the negative control, the amplification curves at 60° C. and 75° C. provided a constant signal. That is, no signal change was detected.

TABLE 11

| | Ct (Cycle threshold) | | |
|---|---|---|---|
| Tube | 60° C. | 70° C. | 95° C. |
| 1 | N/A | 25.97 | N/A |
| 2 | N/A | N/A | N/A |
| 3 | N/A | N/A | N/A |

Tube 1: 5 × 10³ copies of synthetic RNA of SARS-CoV-2 N501Y variant;
Tube 2: 10³ copies of SARS-CoV-2 wild type genomic RNA;
Tube 3: Negative control;
N/A: Not Applicable

These results demonstrate that the L-PTOCE assay and composition according to the present disclosure can be used to distinguish and detect a single nucleotide polymorphism.

### Example 3: Detection of Multiple Target Nucleic Acids

In this Example, it was investigated whether the combination of the L-PTOCE composition and another composition adopting a different signal generation mechanism (i.e., UnderSC-type, InterSC-type, and/or OverSC-type compositions) can detect multiple target nucleic acids in real-time using a single type of label in a single reaction vessel.

First, two target nucleic acids, the genomic DNAs of Mycoplasma Hominis (MH) and genomic DNA of Mycoplasma genitalium (MG), were prepared. The PTOCE assay (WO 2012/096523) using CTO with interactive dual labels, adopting UnderSC-type signal generation mechanism, was used to detect the first target nucleic acid. The L-PTOCE assay according to the present disclosure, adopting InterSC-type signal generation mechanism, was used to detect the second target nucleic acid.

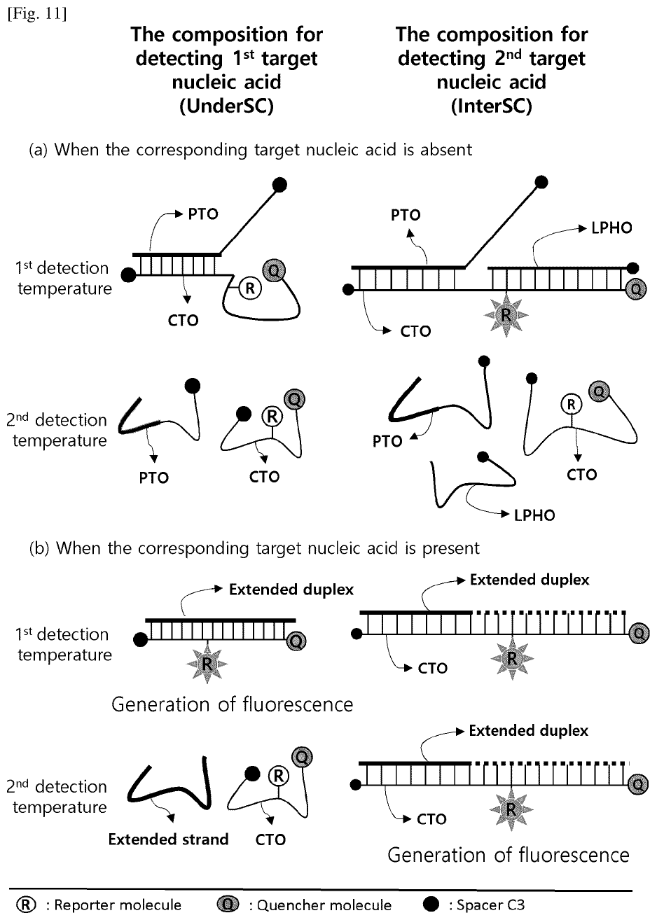

FIG. 11 schematically shows the signal generation principle of the composition for detecting the first target nucleic acid (MH) and the composition for detecting the second target nucleic acid (MG) depending on the presence or absence of the target nucleic acid. According to the present disclosure, by adjusting the respective signal-changing temperature ranges of the PTOCE assay and the L-PTOCE assay, a plurality of target nucleic acids can be detected in real time using a single type of label in one reaction vessel.

Specifically, as shown in FIG. 11, the first composition for detecting the first target nucleic acid, MH, and the second composition for detecting the second target nucleic acid, MG, generate an extended duplex in the presence of the corresponding target nucleic acid, respectively. The sequence and length of the oligonucleotides in the composition for detecting the first target nucleic acid were adjusted such that the extended duplex generated depending on the first target nucleic acid remains its double-stranded form at the first detection temperature and dissociates into a single-stranded state at the second detection temperature. The sequence and length of the oligonucleotides in the composition for detecting the second target nucleic acid were adjusted such that the extended duplex generated depending on the second target nucleic acid remains its double-stranded form at both the first and second detection temperatures while the CTO/LPHO hybrid remains its double-stranded form at the first detection temperature and dissociates into a single-stranded state at the second detection temperature.

As a result, the composition for detecting the first target nucleic acid provides a signal change at the first detection temperature in the presence of the first target nucleic acid, and the signal is constant at the second detection temperature. The composition for detecting the second target nucleic acid provides a constant signal at the first detection temperature in the presence of the second target nucleic acid and provides a change in signal at the second detection temperature.

Both the PTOCE assay for detecting the first target nucleic acid and the L-PTOCE assay for detecting the second target nucleic acid are similar in that they generate extended duplexes depending on the presence of a corresponding target nucleic acid. However, the composition for detecting the first target nucleic acid provides a signal change at the first detection temperature due to the difference between a quenched signal when the first target nucleic acid is absent and an unquenched signal when the first target nucleic acid is present at the first detection temperature, and the composition for detecting the second target nucleic acid provides a signal change at the second detection temperature due to the difference between a quenched signal when the second target nucleic acid is absent and an unquenched signal when the second target nucleic acid is present at the second detection temperature. In addition, the composition for detecting the first target nucleic acid provides a constant signal at the second temperature because there is no difference between the quenched signal when the first target nucleic acid is absent and the quenched signal when the first target nucleic acid is present at the second temperature, and the composition for detecting the second target nucleic acid provides a constant signal at the first temperature because there is no difference between the unquenched signal when the second target nucleic acid is absent and the unquenched signal when the second target nucleic acid is present at the first detection temperature.

### 3-1. Preparation of Target Nucleic Acids and Oligonucleotides

The genomic DNA of Mycoplasma Hominis (MH) (Accession number: ATCC 15488) as the first target nucleic acid, and the genomic DNA of Mycoplasma genitalium (MG) (Accession number: ATCC 33530) as the second target nucleic acid were used.

A first detection temperature for detecting a signal change indicative of the presence of the first target nucleic acid, MH, was set to 60° C., and a second detection temperature for detecting a signal change indicative of the presence of a second target nucleic acid, MG, was set to 75° C. Then, the oligonucleotides for the composition for detecting MH target nucleic acid and the composition for detecting MG target nucleic acid were prepared as below.

For detecting the MH target nucleic acid, a forward primer, a reverse primer, a MH-PTO and a MH-CTO were prepared as shown in Table 12. The MH-PTO comprises in a 5′ to 3′ direction: (i) a 5′-tagging portion comprising a non-hybridizing nucleotide sequence to the MH target nucleic acid and (ii) a 3′-targeting portion comprising a hybridizing nucleotide sequence with the MH target nucleic acid. The MH-CTO comprises in a 3′ to 5′ direction: (i) a capturing portion comprising a hybridizing nucleotide sequence with the 5′-tagging portion of the MH-PTO, and (ii) a templating portion comprising a non-hybridizing nucleotide sequence with the 5′-tagging portion and the 3′-targeting portion of the MH-PTO. The MH-CTO has a quencher molecule (BHQ-1) linked to the 5′-end and a reporter molecule (CAL Fluor Orange 560) linked to the 3′-targeting portion. The 3′-ends of the PTO and CTO were each blocked by Spacer C3 to prohibit its extension by DNA polymerase.

The oligonucleotides for detecting the second target nucleic acid, MG, were the same as those used in Example 1 for MG target nucleic acid detection, including the forward primer (SEQ ID NO: 1), reverse primer (SEQ ID NO: 2), PTO (SEQ ID NO: 3), CTO-1 (SEQ ID NO: 4), and LPHO-1A (SEQ ID NO: 5).

TABLE 12

| Target | | | | |
|---|---|---|---|---|
| nucleic | SEQ ID | Oligo | Tm | |
| acid | No. | Type | (° C.) | Sequence (5′-3′) |
| MH | 16 | MH- | 65 | GCTTCATGTACTACTAACTGTTTAGCIIIIITTGCCAAC |
| | | Forward | | GT |
| | | primer | | |
| | 17 | MH- | 63 | TCCAATAGCTATTGCAGCACCIIIIITTGTTGGAAC |
| | | Reverse | | |
| | | primer | | |
| | 18 | MH-PTO | 71 | ATATCGCGCGTCTGCTCCACACAAAGATTTAAGAAG |
| | | | | AGCAAG [Spacer C3] |
| | 19 | MH-CTO | 66 | [BHQ-1]TTTATTTATTTATTTAT[T(CAL Fluor Orange |
| | | | | 560)]TTACTGCAGACGCGCGATAT[Spacer C3] |

The underlined character indicates the 5′-tagging portion of the PTO.

### 3-2. Multiplex Real-Time PCR

Multiplex real-time PCR was performed in one reaction vessel using the above oligonucleotides.

The target nucleic acids (Tube 1:500 fg of MH genomic DNA; Tube 2:1 μg of MG genomic DNA; Tube 3:500 fg of MH genomic DNA and 1 μg of MG genomic DNA) and distilled water (Tube 4, Negative control) were mixed with: 5 pmole of MH-forward primer (SEQ ID NO: 16), 5 pmole of MH-reverse primer (SEQ ID NO: 17), 3 pmole of MH-PTO (SEQ ID NO: 18), 1 pmole of MH-CTO (SEQ ID NO: 19), 5 pmole of MG-forward primer (SEQ ID NO: 1), 5 pmole of MG-reverse primer (SEQ ID NO: 2), 3 pmole of MG-PTO (SEQ ID NO: 3), 1 pmole of CTO-1 (SEQ ID NO: 4) and 3 pmole LPHO-1A (SEQ ID NO: 5), and then combined with 5 μL of 4X Enzyme Mix (20 U of Taq DNA polymerase) (Nanohelix, Korea) and 5 μL of 4X Buffer mix (final, 0.8 mM of dNTPs, 50 mM of KCl, 3.5 mM of MgCl₂) (Nanohelix, Korea), to prepare a reaction mixture in the final volume of 20 μL.

The Tubes 1 to 4 containing the reaction mixture were each placed in a real-time thermal cycler (CFX96 Real-time Cycler, Bio-Rad) and subjected to real-time PCR consisting of denaturation at 95° C. for 15 minutes, and then 50 cycles of 10 seconds at 95° C., 15 seconds at 60° C., 10 seconds at 72° C., and 5 seconds at 75° C. Detection of the signal was performed at 60° C. for detecting the MH target nucleic acid and at 75° C. for detecting the MG target nucleic acid in each cycle.

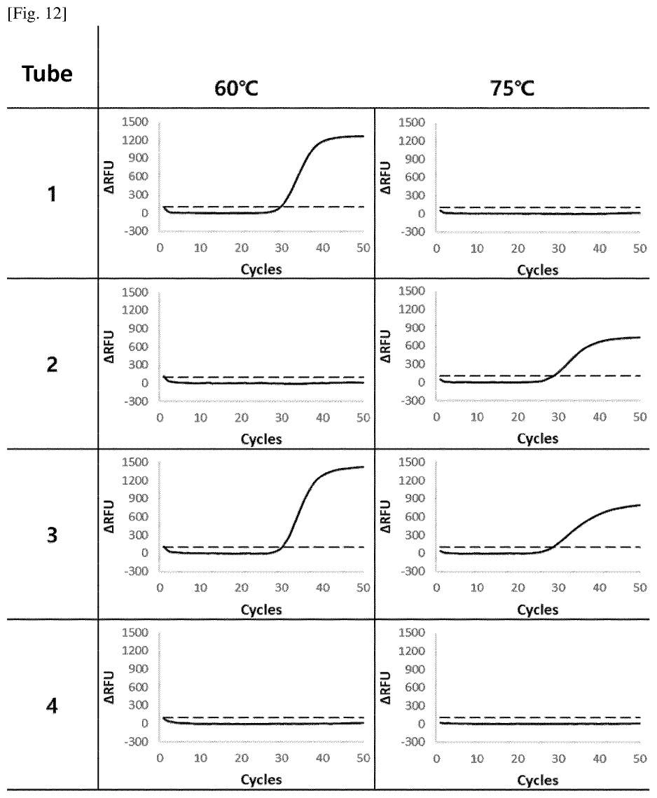

The real-time PCR results were shown in FIG. 12.

For Tube 1 containing only MH target nucleic acid, the amplification curve at 75° C. exhibited a constant signal even though the MH target nucleic acid was amplified. In contrast, the amplification curve at 60° C. exhibited a signal change as the MH target nucleic acid was amplified. For Tube 2 containing only MG target nucleic acid, the amplification curve at 60° C. exhibited a constant signal even though the MG target nucleic acid was amplified. In contrast, the amplification curve at 75° C. exhibited a signal change as the MG target nucleic acid was amplified. For Tube 3 containing the MH and MG target nucleic acids, the amplification curves at 60° C. and 75° C. exhibited a signal change, respectively.

Meanwhile, for Tube 4 as the negative control, the amplification curves at 60° C. and 75° C. provided a constant signal. That is, no signal change was detected.

These results demonstrate that the combination of the L-PTOCE composition and another composition for target nucleic acid detection adopting a different signal generation mechanism (i.e., UnderSC-type, InterSC-type, and/or OverSC-type compositions) can be used to detect multiple target nucleic acids in real-time using a single type of label in a single reaction vessel. Furthermore, it would be appreciated that a combination of multiple L-PTOCE compositions, each of which is an InterSC-type composition, can be used to detect multiple target nucleic acids in real-time.

In an alternative method, the change in signal was measured using the reference signal value obtained from the negative control reaction. The signal was considered to have changed if the signal values at 60° C. and 75° C. were higher than RFU 100, a threshold based on the signal value of the negative control reaction (i.e., RFU: 0). As a result, as shown in Table 13, a signal change in Tube 1 was identified from a Ct value of 29.37 only at 60° C., a signal change in Tube 2 was identified from a Ct value of 28.49 only at 75° C. and signal changes in Tube 3 were identified from a Ct value of 29.61 and a Ct value of 28.39 at 60° C. and 75° C., respectively. On the other hand, for tube 4 as the negative control, no change in signal was identified at both detection temperatures.

TABLE 13

Ct (Cycle threshold)

| | first detection | second detection |
|---|---|---|
| Tube | temperature (60° C.) | temperature (75° C.) |
| 1 | 29.37 | N/A |
| 2 | N/A | 28.49 |
| 3 | 29.61 | 28.39 |
| 4 | N/A | N/A |

Tube 1: 500 fg of MH genomic DNA;
Tube 2: 1 pg of MG genomic DNA;
Tube 3: 500 fg of MH genomic DNA and 1 pg of MG genomic DNA;
Tube 4: Negative control;
N/A: Not Applicable

In summary, the L-PTOCE assay according to the present disclosure can detect one or more target nucleic acids using the same type of label in one reaction vessel.

Having described a preferred embodiment of the present invention, it is to be understood that variants and modifications thereof falling within the spirit of the invention may become apparent to those skilled in this art. Therefore, the scope of the present invention shall be determined by the appended claims and their equivalents.

1. A method for detecting a target nucleic acid in a sample by LPHO-assisted PTO Cleavage and Extension (L-PTOCE) assay, comprising:
(a) hybridizing a primer and a Probing and Tagging Oligonucleotide (PTO) with the target nucleic acid;
wherein the primer comprises a hybridizing nucleotide sequence with a first region of the target nucleic acid,
wherein the PTO comprises in a 5′ to 3′ direction: (i) a 5′-tagging portion, and (ii) a 3′-targeting portion,
wherein the 3′-targeting portion comprises a hybridizing nucleotide sequence with a second region of the target nucleic acid, and the 5′-tagging portion comprises a nucleotide sequence not hybridized with the target nucleic acid when the 3′-targeting portion is hybridized with the second region of the target nucleic acid,
wherein the primer is located upstream of the PTO;
(b) contacting the resultant of the step (a) to a DNA polymerase having 5′ nuclease activity under conditions for cleavage of the PTO;
wherein the primer is extended to induce cleavage of the PTO by the DNA polymerase having 5′ nuclease activity such that the cleavage releases a fragment comprising the 5′-tagging-portion of the PTO;
(c) hybridizing the fragment released from the PTO with a Capturing and Templating Oligonucleotide (CTO);
wherein the CTO comprises in a 3′ to 5′ direction: (i) a capturing portion comprising a hybridizing nucleotide sequence with the 5′-tagging portion of the PTO, and (ii) a templating portion comprising a non-hybridizing nucleotide sequence with the 5′-tagging portion and the 3′-targeting portion of the PTO,
wherein the CTO has a reporter molecule and a quencher molecule defining a labeled portion,
wherein the fragment is hybridized with the capturing portion of the CTO;
(d) performing an extension reaction using the resultant of the step (c) and the DNA polymerase having 5′ nuclease activity in the presence of a Labeled Portion Hybridizing Oligonucleotide (LPHO);
wherein the LPHO comprises a hybridizing nucleotide sequence with the labeled portion of the CTO,
wherein when the target nucleic acid is present in the sample, the fragment hybridized with the capturing portion of the CTO is extended to generate an extended strand complementary to the CTO, thereby generating an extended duplex between the extended strand and the CTO,
wherein the generation of the extended duplex prevents the formation of a CTO/LPHO hybrid between the labeled portion of the CTO and the LPHO,
wherein when the target nucleic acid is not present in the sample, the extended strand is not generated, and instead the CTO/LPHO hybrid is formed,
wherein the extended duplex has a melting temperature (Tm) that is different from a Tm of the CTO/LPHO hybrid; and
(e) detecting the presence of the extended duplex;
wherein the extended duplex is detected by measuring a signal provided from the extended duplex,
wherein the measurement is performed at a temperature at which the signal intensity provided from the extended duplex is different from a signal intensity provided from the CTO/LPHO hybrid, and
wherein the presence of the extended duplex indicates the presence of the target nucleic acid.

2. The method of claim 1, wherein the extended duplex is generated by (i) extending the fragment hybridized to the capturing portion of the CTO prior to the hybridization of the labeled portion of the CTO and the LPHO, (ii) extending the fragment hybridized to the capturing portion of the CTO upon the hybridization between the labeled portion of the CTO and the LPHO, thereby cleaving the LPHO, or (iii) both (i) and (ii).

3. The method of claim 2, wherein the generation of the extended duplex prevents the formation of the CTO/LPHO hybrid, via preference for hybridization between the extended strand and the CTO over hybridization between the labeled portion of the CTO and the LPHO.

4. The method of claim 2, wherein the generation of the extended duplex prevents the formation of the CTO/LPHO hybrid, via cleavage of the LPHO during the extension of step (d).

5. The method of claim 1, wherein when the CTO is unhybridized with the extended strand or the LPHO, the reporter molecule and the quencher molecule on the CTO are in close proximity to each other, thereby causing the quencher molecule to quench a signal from the reporter molecule.

6. The composition of claim 5, wherein when the CTO is hybridized with the extended strand or the LPHO, the reporter molecule and the quencher molecule on the CTO are separated, thereby causing the quencher molecule to unquench a signal from the reporter molecule.

7. The method of claim 1, wherein (i) both the reporter molecule and the quencher molecule are linked to the capturing portion of the CTO, (ii) both the reporter molecule and the quencher molecule are linked to the templating portion of the CTO, or (iii) one of the reporter molecule and the quencher molecule is linked to the capturing portion of the CTO and the other is linked to the templating portion of the CTO.

8. The method of claim 1, wherein the LPHO is hybridized with a complete or a partial sequence of the labeled portion of the CTO, and the reporter molecule and the quencher molecule on the CTO are separated, thereby causing the quencher molecule to unquench a signal from the reporter molecule.

9. The method of claim 8, wherein the Tm of the extended duplex is at least 3° C. higher than the Tm of the CTO/LPHO hybrid.

10. The method of claim 1, wherein the Tm of the extended duplex is adjustable by (i) a sequence and/or length of the fragment, (ii) a sequence and/or length of the CTO or (iii) the sequence and/or length of the fragment and the sequence and/or length of the CTO, and the Tm of the CTO/LPHO hybrid is adjustable by a sequence and/or length of the LPHO.

11. The method of claim 1, wherein the LPHO comprises a nucleotide sequence which competes with the fragment for hybridization with the CTO.

12. The method of claim 11, wherein the LPHO is not cleaved by the fragment or its extension product.

13. The method of claim 1, wherein the LPHO comprises a nucleotide sequence which does not compete with the fragment for hybridization with the CTO.

14. The method of claim 13, wherein the LPHO is cleaved by the fragment or its extension product.

15. The method of claim 1, wherein the temperature for measurement depends on the Tm of the extended duplex and the Tm of the CTO/LPHO hybrid.

16. The method of claim 1, wherein the method is performed in the presence of a plurality of the PTOs, a plurality of the CTOs, and a plurality of the LPHOs, and the steps (a)-(e) are repeated with denaturation between repeating cycles.

17. The method of claim 16, wherein the temperature for measurement allows both (i) at least one of the extended duplexes to remain its double-stranded state and (ii) at least one of the CTO/LPHO hybrids to dissociate into a single-stranded state.

18-52. (canceled)
